(19) United States
(12) Patent Application Publication
(10) Pub. No.: US 2026/0295080 A1
(43) Pub. Date: Oct. 1, 2026

(54) LIPIDS AND NANOPARTICLE FORMULATIONS THEREOF

(71) Applicant: Arcturus Therapeutics, Inc., San Diego, CA (US)

(72) Inventors: Kumar Rajappan, San Diego, CA (US); Steven P. Tanis, San Diego, CA (US); Sean S. Sullivan, San Diego, CA (US)

(21) Appl. No.: 19/457,470

(22) Filed: Jan. 23, 2026

Related U.S. Application Data
(60) Provisional application No. 63/748,755, filed on Jan. 23, 2025.

(51) Int. Cl. A61K 48/00; A61K 9/1272; A61K 9/19; A61K 9/51; A61K 31/7105; A61K 47/02; A61K 47/10; A61K 47/22; A61K 47/26; C07F 9/10; C07J 9/00; C07J 31/00; C07J 43/00; C12N 15/113; C12N 15/88

(52) U.S. Cl. CPC A61K 48/0041; A61K 9/1272; A61K 9/19; A61K 9/5123; A61K 31/7105; A61K 47/02; A61K 47/10; A61K 47/22; A61K 47/26; C07F 9/10; C07J 9/00; C07J 31/003; C07J 43/003; C12N 15/113; C12N 15/88; C12N 2310/14

(57) ABSTRACT

The present disclosure relates to compounds of Formula I, or a pharmaceutically acceptable salt thereof, pharmaceutical compositions thereof, and methods thereof.

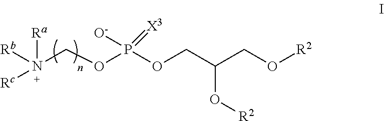

### CROSS-REFERENCE TO RELATED APPLICATIONS

This application claims priority to U.S. Provisional Application No. 63/748,755, filed Jan. 23, 2025, the contents of which are hereby incorporated by reference in their entirety.

### FIELD

The present disclosure relates generally to lipids. For example, the present disclosure relates to synthetic lipids and methods and uses thereof in the formulation and delivery of nucleic acids and therapeutic molecules.

### BACKGROUND

The variety of nucleic acid-based therapeutics for targeted delivery creates a challenge for lipid-based delivery vehicles. For example, nucleic acids are structurally diverse in size and type. Examples include DNA used in gene therapy, plasmids, small interfering nucleic acids (siRNA), and microRNA (miRNA) for use in RNA interference (RNAi), antisense molecules, ribozymes, antagomirs, and aptamers.

The design and use of cationic lipids and ionizable cationic lipids for inclusion in such lipid-based delivery vehicles has shown great advantages. However, use of these lipids can contribute to significant side effects when administered in vivo. One problem that has been observed includes low biodegradability and clearance from target tissues, thus creating an in vivo buildup of the lipid. Another problem is that large amounts of the lipid may cause an adverse immunogenic effect, which can result in discomfort in the subject and a decrease in the therapeutic effect of the active ingredient. A third problem associated with many cationic lipids is a low percentage of effective delivery to the target, thus resulting in a relatively low therapeutic effect or low potency. Moreover, it is not only important that the cationic lipid in the delivery vehicle have a specially tuned pKa so it can formulate with the nucleic acid-based therapeutic agent and protect it from degradation during administration, but be able to release the therapeutic agent once the vehicle has reached its target. With any drug discovery and development paradigms, there needs to be a continuous effort to understand the pharmacology, pharmacokinetics, and toxicology so that drugs with optimum therapeutic index and regulatory approval potential can be initiated into clinical investigations. As room for chemical optimization of nucleic acid is limited, there is a need for optimization of the lipid component of the delivery system.

Thus, there is a need in the art for the development of lipids that can meet the special needs of lipid-nucleic acid delivery systems.

### SUMMARY

As shown herein, the lipid compounds of the present disclosure are able to form stable lipid formulations such as lipid nanoparticles (LNPs) for use in delivering nucleic acid such as mRNA and siRNA, for therapeutic applications such as protein expression in various species.

In one aspect, the present disclosure provides a compound of Formula I:

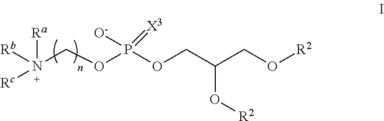

wherein
X¹, X², and X³ are each independently O or S,
R^{a}, R^{b}, and R^{c} are each independently H, or C₁₋₆ alkyl, or
R^{a} is H or C₁₋₆ alkyl, and R^{b} and R^{c} taken together with the N they are attached to form a 3- to 8-membered heterocyclyl, the heterocyclyl being optionally substituted with one or more C₁₋₃ alkyl,
n is 2 to 6,
either
   (i) R¹ is

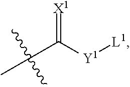

and R² is

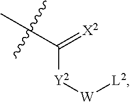

wherein
   Y¹ is absent, O, or S,
   L¹ is C₃₋₄₀ alkyl, C₃₋₄₀ alkenyl, or C₃₋₄₀ alkynyl, either
      (a) Y² is absent or C₁₋₄ alkylene, W is absent, O, or S, and L² is selected from C₃₋₄₀ alkyl, C₃₋₄₀ alkenyl, and C₃₋₄₀ alkynyl, optionally the alkyl, alkenyl, and alkynyl are branched, or
      (b) Y² is C₁₋₄ alkylene, optionally substituted with oxo, W is absent, and L² is cholesterol or cholesterol derivative attached to Y² via an O atom of the cholesterol or via an O or S atom of the cholesterol derivative; or
(ii) R¹ is

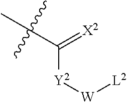

and R² is

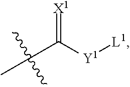

wherein
   Y¹ is absent, O, or S,
   L¹ is C₃₋₄₀ alkyl, C₃₋₄₀ alkenyl, or C₃₋₄₀ alkynyl,
   Y² is C₁₋₄ alkylene, W is absent or carbonyl, and L² is cholesterol or cholesterol derivative attached to Y² via an O atom of the cholesterol or via an O or S atom of the cholesterol derivative.

In some embodiments, the compound of Formula I has a structure of Formula I-I:

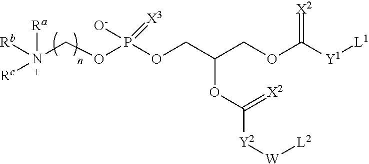

or a pharmaceutically acceptable salt thereof,
wherein
   X¹, X², and X³ are each independently O or S,
   R^{a}, R^{b}, and R^{c} are each independently H, or C₁₋₆ alkyl, or
   R^{a} is H or C₁₋₆ alkyl, and R^{b} and R^{c} taken together with the N they are attached to form a 3- to 8-membered heterocyclyl, the heterocyclyl being optionally substituted with one or more C₁₋₃ alkyl,
   n is 2 to 6,
   Y¹ is absent, O, or S,
   L¹ is C₃₋₄₀ alkyl, C₃₋₄₀ alkenyl, or C₃₋₄₀ alkynyl,
   Y² is absent or C₁₋₄ alkylene, W is absent, O, or S, and L² is selected from C₃₋₄₀ alkyl, C₃₋₄₀ alkenyl, and C₃₋₄₀ alkynyl, optionally the alkyl, alkenyl, and alkynyl are branched, or
   Y² is C₁₋₄ alkylene, optionally substituted with oxo, W is absent, and L² is cholesterol or cholesterol derivative attached to Y² via an O atom of the cholesterol or via an O or S atom of the cholesterol derivative.

In another aspect, the present disclosure provides a lipid composition comprising a nucleic acid and a compound of the present disclosure.

In another aspect, the present disclosure provides a lipid nanoparticle comprising a compound of the present disclosure. In some embodiments, the compound of the present disclosure self-assembles to form the lipid nanoparticle.

In another aspect, the present disclosure provides a pharmaceutical composition comprising a compound of the present disclosure, or a lipid composition of the present disclosure or a lipid nanoparticle of the present disclosure, and a pharmaceutically acceptable excipient.

In another embodiment, the present disclosure provides a method of treating and/or preventing a disease in a subject in need thereof, the method comprising administering to the subject a therapeutically effective amount of a compound of the present disclosure, or a pharmaceutically acceptable salt thereof, a pharmaceutical composition of the present disclosure, a lipid composition of the present disclosure or a lipid nanoparticle of the present disclosure.

In another embodiment, the present disclosure provides a method of expressing a protein or polypeptide in a target cell, comprising contacting the target cell with a lipid nanoparticle of the present disclosure, or the pharmaceutical composition of the present disclosure.

In another embodiment, the present disclosure provides a compound of the present disclosure, or a lipid composition of the present disclosure or a lipid nanoparticle of the present disclosure for use in treating or preventing a disease in a subject in need thereof.

In another embodiment, the present disclosure provides a use of a compound of the present disclosure, or a lipid composition of the present disclosure or a lipid nanoparticle of the present disclosure for treating and/or preventing a disease in a subject in need thereof.

In another embodiment, the present disclosure provides a use of a compound of the present disclosure, or a lipid composition of the present disclosure or a lipid nanoparticle of the present disclosure in the preparation of a medicament for treating and/or preventing a disease in a subject in need thereof.

In another embodiment, the present disclosure provides the compound of the present disclosure, or a pharmaceutically acceptable salt thereof, for use in therapy.

### BRIEF SUMMARY OF DRAWINGS

FIG. 1 shows a graph of the percent reduction of FVII plasma protein (as a percentage of base value) at 48 hours after mice are administered intravenously with FVII siRNA encapsulated LNPs at dose levels of 0.01 and 0.03 mg/Kg of FVII siRNA.

FIG. 2A. shows a bar graph of mouse serum hEPO protein expressions (ng/mL) at 6 hours post intravenous administration of hEPO mRNA in LNPs formulated with DSPC, DMPC, DPPC and DAPC each as helper lipids and L1 as the principal ionizable cationic lipid (iCL) at 0.3 mg/Kg of hEPO mRNA. FIG. 2B. shows a bar graph of hEPO expressions in NHP serum at 0.3 mg/kg dose of hEPO mRNA encapsulated with DSPC, DMPC, DPPC and DAPC each as helper lipids and L1 as the principal iCL (PBS as negative control).

FIG. 3 shows a bar graph of comparative mouse serum hEPO protein levels 6 h post administration of 0.3 mg/Kg of hEPO mRNA formulated with ionizable lipid L1 in combination with compound I-2, I-5, I-6, I-7, I-8 or I-9. DSPC formulation was used as the comparator. PBS was used as negative control.

FIG. 4 shows a bar graph of mouse serum hEPO protein expressions (ng/mL) at 6 hours post intravenous administration of hEPO mRNA in LNPs formulated with DSPC, I-2 or I-3 and L1 as the principal iCL at 0.1 or 0.3 mg/Kg of hEPO mRNA. PBS was used as negative control.

FIG. 5 shows a bar graph of hEPO expressions in NHP serum at 0.3 mg/kg dose of hEPO mRNA encapsulated with DSPC, I-2 or I-3, and L1 as the principal iCL. PBS was used as negative control.

FIG. 6A shows a bar graph of relative expression of hEPO in NHPs treated with LNPs formulated with compound I-2 with ionizable lipid L1 (n=3), 0.3 mg/Kg of hEPO mRNA. FIG. 6B shows a bar graph of relative expression of hEPO in NHPs treated with LNPs formulated with compound I-2 with different ionizable lipids (L8, L9) (n=3), 0.3 mg/Kg of hEPO mRNA. FIG. 6C shows a bar graph of relative expression of hEPO in NHPs treated with LNPs formulated with compound I-2 with different ionizable lipids (L10, L11) (n=3), 0.3 mg/Kg of hEPO mRNA. FIG. 6D shows a bar graph of relative expression of hEPO in NHPs treated with LNPs formulated with compound I-2 with different ionizable lipids (L12, L13) (n=3), 0.3 mg/Kg of hEPO mRNA. FIG. 6E shows a bar graph of relative expression of hEPO in NHPs treated with LNPs formulated with compound I-2 with different ionizable lipids (L14, L15) (n=3), 0.3 mg/Kg of hEPO mRNA. FIG. 6F shows a bar graph of relative expression of hEPO in NHPs treated with LNPs formulated with compound I-2 with different ionizable lipids (L16, L17) (n=3), 0.3 mg/Kg of hEPO mRNA.

### DETAILED DESCRIPTION

### I. General

The disclosure relates generally to methods and compounds, and pharmaceutically acceptable salts thereof, for intracellular delivery of therapeutic molecules such as nucleic acids. Specifically, the present disclosure relates to compounds of Formula I and pharmaceutically acceptable salts thereof, and compositions thereof. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

A squiggly line on a chemical group as shown below, for example,

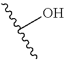 [SMILES: *O]

indicates a point of attachment, i.e., it shows the broken bond by which the group is connected to another described group.

As used herein, “a compound of the present disclosure” can mean a compound of any of Formula I, or a pharmaceutically acceptable salt thereof. Similarly, the phrase “a compound of Formula (number)” means a compound of that formula and pharmaceutically acceptable salts thereof.

The prefix “Cᵤ₋ᵥ” and “Cᵤ-Cᵥ” indicates that the following group has from u to v carbon atoms. For example, “C₁₋₈ alkyl” and “C₁-C₈ alkyl” indicates that the alkyl group has from 1 to 8 carbon atoms.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadcyl, hexadecyl, heptadecyl and octadecyl.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or —OEt), isopropoxy (—O—CH(CH₃)₂), t-butoxy (—O—C(CH₃)₃ or —OtBu) and the like. Other examples of suitable alkoxy groups include, but are not limited to, sec-butoxy, tert-butoxy, pentoxy, hexoxy, and the like.

“Alkoxyalkyl” refers an alkoxy group linked to an alkyl group which is linked to the remainder of the compound. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). Alkoxy and alkyl are as defined above. Examples of “alkoxyalkyl” include, but are not limited to, methoxymethyl (CH₃OCH₂—), and methoxyethyl (CH₃OCH₂CH₂).

“Bridged” means a ring system in which non-adjacent atoms on a ring are connected by a divalent substituent, such as an alkylenyl or heteroalkylenyl group or a single heteroatom.

“Hydroxyalkyl” refers to a hydroxy group, —OH, linked to an alkyl group which is linked to the remainder of the compound such that the alkyl group is divalent. Hydroxyalkyl can have any suitable number of carbons, such as from 1 to 8 (C₁₋₈ hydroxyalkyl), 1 to 6 (C₁₋₆ hydroxyalkyl), 2 to 6 (C₂₋₆ hydroxyalkyl), 2 to 4 (C₂₄ hydroxyalkyl), or 2 to 3 (C₂₋₃ hydroxyalkyl). Alkyl is as defined above where the alkyl is divalent.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁₋₂₀ haloalkyl), 1 to 12 carbon atoms (i.e., C₁₋₁₂ haloalkyl), 1 to 8 carbon atoms (i.e., C₁₋₈ haloalkyl), 1 to 6 carbon atoms (i.e., C₁₋₆ haloalkyl) or 1 to 3 carbon atoms (i.e., C₁₋₃ haloalkyl). The alkyl groups can be substituted with 1, 2, 3, 4, 5, 6, 7, 8, 9 or more halogens. Examples of suitable haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, 4, 5, 6, 7, 8, 9 or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Heteroalkyl” refers to an unbranched or branched saturated hydrocarbon chain containing from 1 to 4 heteroatoms.

“Cycloalkyl” refers to a saturated or partially saturated cyclic alkyl group having a single ring or multiple rings, such as 2, 3, 4 or more, wherein the multiple rings can be fused, bridged, spiro, or any combination thereof. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

The term “fused” refers to a ring system in which two or more rings in the system share a pair of adjacent ring atoms.

“Spiro” refers to at least two rings are linked together by one common atom. “Spiro” also refers to a ring substituent which is joined by two bonds at the same carbon atom. Examples of spiro groups include, but are not limited to, 1,1-diethylcyclopentane, dimethyl-dioxolane, and 4-benzyl-4-methylpiperidine, wherein the cyclopentane and piperidine, respectively, are the spiro substituents.

“Heterocycle” or “heterocyclyl” or “heterocycloalkyl” refer to a saturated or unsaturated cyclic alkyl group, with one or more ring heteroatoms independently selected from nitrogen, oxygen, sulfur and silicon. A heterocyclyl can be a single ring or multiple rings, such as 2, 3, 4 or more, wherein the multiple rings can be fused, bridged, spiro, or any combination thereof. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, tetrahydropyridinyl, piperazinyl, oxetanyl, dihydropyranyl, dioxolanyl, azetidinyl, and morpholinyl.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅-6. In some instances, the alkyl component can be absent. The aryl component is as defined above. Examples of alkyl-aryl groups include, but are not limited to, benzyl and ethyl-benzene.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21st Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Tomography (PET) studies for examining substrate receptor occupancy. Isotopically-labeled compounds of Formulas I and II, can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1.1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. A prefix such as “Cᵤ₋ᵥ”, “Cᵤ-Cᵥ” or “(Cᵤ-Cᵥ)” indicates that the following group has from u to v carbon atoms. For example, “C₁₋₆ alkyl” and “C₁-C₆ alkyl” both indicate that the alkyl group has from 1 to 6 carbon atoms.

Unless otherwise specified, the carbon atoms of the compounds of Formula I, are intended to have a valence of four. If in some chemical structure representations, carbon atoms do not have a sufficient number of variables attached to produce a valence of four, the remaining carbon substituents needed to provide a valence of four should be assumed to be hydrogen.

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

“Disease” or “condition” refer to a state of being or health status of a patient or subject capable of being treated with a compound, pharmaceutical composition, or method provided herein. The disease may be an autoimmune, inflammatory, cancer, infectious (e.g., a viral infection), metabolic, developmental, cardiovascular, liver, intestinal, endocrine, neurological, or other disease. In some embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

The term “adjacent carbons” and “adjacent atoms” as used herein refers to consecutive carbon atoms that are directly attached to each other. For example, in

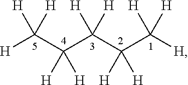

C₁ and C₂ are adjacent carbons, C₂ and C₃ are adjacent carbons, C₃ and C₄ are adjacent carbons, and C₄ and C₅ are adjacent carbons. Similarly, in

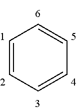

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

The term “double bond” as used herein refers to the formation of an additional single bond between two adjacent atoms that are already connected by a single bond, thus forming a double bond. For example, in

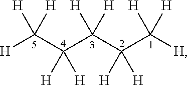

C₁ and C₂ are adjacent carbons, C₂ and C₃ are adjacent carbons, C₃ and C₄ are adjacent carbons, and C₄ and C₅ are adjacent carbons, such that two hydrogens, or R groups, on adjacent atoms are combined with the atoms to which they are attached and the bond linking the adjacent atoms to form a double bond as shown in the following:

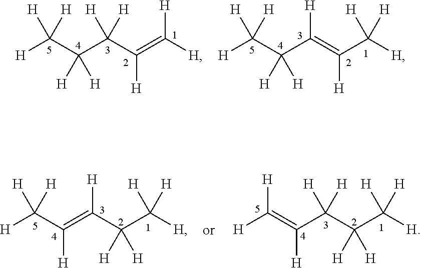

### Similarly, in

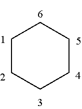

C₁ and C₂ are adjacent carbons, C₂ and C₃ are adjacent carbons, C₃ and C₄ are adjacent carbons, C₄ and C₅ are adjacent carbons, C₅ and C₆ are adjacent carbons, and C₆ and C₁ are adjacent carbons, such that two hydrogens, or R groups, on adjacent atoms are combined with the atoms to which they are attached and the bond linking the adjacent atoms to form a double bond as shown in the following:

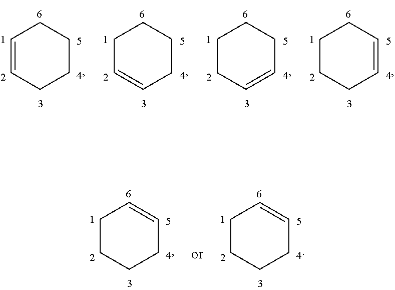

The term “non-adjacent carbons” and “non-adjacent atoms” as used herein refers to non-consecutive carbons atoms that are not directly attached to each other. For example, in

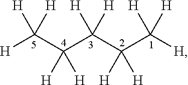

C₁ and C₃ are non-adjacent carbons, C₁ and C₄ are non-adjacent carbons, C₂ and C₄ are non-adjacent carbons, C₁ and C₅ are non-adjacent carbons, C₂ and C₅ are non-adjacent carbons, and C₃ and C₅ are non-adjacent carbons, among others. Similarly, in

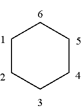

C₁ and C₃ are non-adjacent carbons, C₁ and C₄ are non-adjacent carbons, C₂ and C₄ are non-adjacent carbons, C₁ and C₅ are non-adjacent carbons, C₂ and C₅ are non-adjacent carbons, C₃ and C₅ are non-adjacent carbons, C₂ and C₆ are non-adjacent carbons, C₃ and C₆ are non-adjacent carbons, and C₄ and C₆ are non-adjacent carbons.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

### III. Compounds

### Compound of Formula I

Disclosed herein are, among other things, compounds of Formula I.

In one aspect, the present disclosure provides a compound of Formula I:

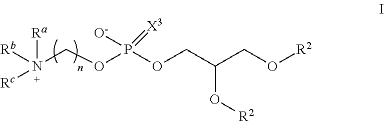

wherein
X¹, X², and X³ are each independently O or S,
R^{a}, R^{b}, and R^{c} are each independently H, or C₁₋₆ alkyl, or
R^{a} is H or C₁₋₆ alkyl, and R^{b} and R^{c} taken together with the N they are attached to form a 3- to 8-membered heterocyclyl, the heterocyclyl being optionally substituted with one or more C₁₋₃ alkyl,
n is 2 to 6,
either
   (i) R¹ is

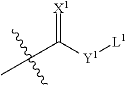

and R² is

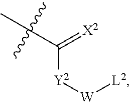

wherein
   Y¹ is absent, O, or S,
   L¹ is C₃₋₄₀ alkyl, C₃₋₄₀ alkenyl, or C₃₋₄₀ alkynyl,
   either
      (a) Y² is absent or C₁₋₄ alkylene, W is absent, O, or S, and L² is selected from C₃₋₄₀ alkyl, C₃₋₄₀ alkenyl, and C₃₋₄₀ alkynyl, optionally the alkyl, alkenyl, and alkynyl are branched, or
      (b) Y² is C₁₋₄ alkylene, optionally substituted with oxo, W is absent, and L² is cholesterol or cholesterol derivative attached to Y² via an O atom of the cholesterol or via an O or S atom of the cholesterol derivative; or
(ii) R¹ is

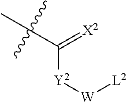

and R² is

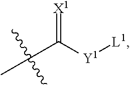

wherein
   Y¹ is absent, O, or S,
   L¹ is C₃₋₄₀ alkyl, C₃₋₄₀ alkenyl, or C₃₋₄₀ alkynyl,
   Y² is C₁₋₄ alkylene, W is absent or carbonyl, and L² is cholesterol or cholesterol derivative attached to Y² via an O atom of the cholesterol or via an O or S atom of the cholesterol derivative.

In some embodiments, the compound of Formula I has a structure of Formula I-I:

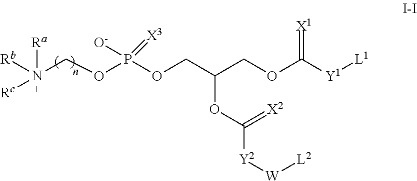

or a pharmaceutically acceptable salt thereof,
wherein
   X¹, X², and X³ are each independently O or S,
   R^{a}, R^{b}, and R^{c} are each independently H, or C₁₋₆ alkyl, or
   R^{a} is H or C₁₋₆ alkyl, and R^{b} and R^{c} taken together with the N they are attached to form a 3- to 8-membered heterocyclyl, the heterocyclyl being optionally substituted with one or more C₁₋₃ alkyl,
   n is 2 to 6,
   Y¹ is absent, O, or S,
   L¹ is C₃₋₄₀ alkyl, C₃₋₄₀ alkenyl, or C₃₋₄₀ alkynyl, Y² is absent or C₁₋₄ alkylene, W is absent, O, or S, and L² is selected from C₃₋₄₀ alkyl, C₃₋₄₀ alkenyl, and C₃₋₄₀ alkynyl, optionally the alkyl, alkenyl, and alkynyl are branched, or
   Y² is C₁₋₄ alkylene, optionally substituted with oxo, W is absent, and L² is cholesterol or cholesterol derivative attached to Y² via an O atom of the cholesterol or via an O or S atom of the cholesterol derivative.

In some embodiments, X¹, X², and X³ are each independently O.

In some embodiments, R^{a} is H. In some embodiments, R^{b} and R^{c} are each independently C₁₋₆ alkyl. In some embodiments, R^{a} is H, and R^{b} and R^{c} are each independently C₁₋₆ alkyl. In some embodiments, R^{a} is C₁₋₆ alkyl. In some embodiments, R^{a}, R^{b} and R^{c} are each independently C₁₋₆ alkyl.

In some embodiments, R^{a}, R^{b}, and R^{c} are each independently C₁₋₃ alkyl. In some embodiments, R^{a}, R^{b}, and R^{c} are each independently methyl.

In some embodiments, R^{a} is C₁₋₆ alkyl, and R^{b} and R^{c} taken together with the N they are attached to form a 3- to 8-membered heterocyclyl, optionally substituted with one or more C₁₋₃ alkyl. In some embodiments, when R^{b} and R^{c} taken together with the N they are attached to form a 3- to 8-membered heterocyclyl, R^{a} is C₁₋₃ alkyl. In some embodiments, when R^{b} and R^{c} taken together with the N they are attached to form a 3- to 8-membered heterocyclyl, R^{a} is methyl.

In some embodiments, R^{b} and R^{c} taken together with the N they are attached to form a piperazinyl. Optionally, the piperazinyl is substituted with one or more C₁₋₃ alkyl.

In some embodiments, n is 2 to 4. In some embodiments, n is 2.

In some embodiments, Y¹ is absent. In some embodiments, Y¹ is O or S.

In some embodiments, L¹ is C₆₋₂₅ alkyl, C₆₋₂₅ alkenyl, or C₆₋₂₅ alkynyl. In some embodiments, L¹ is C₆₋₂₅ alkyl. In some embodiments, L¹ is C₁₀₋₂₀ alkyl. In some embodiments, L¹ is C₆₋₂₅ alkenyl. In some embodiments, L¹ is C₆₋₂₅ alkynyl.

In some embodiments, L¹ is branched. In some embodiments, L¹ is unbranched.

In some embodiments, Y² is absent or C₁₋₄ alkylene, W is absent, O, or S, and L² is selected from C₃₋₄₀ alkyl, C₃₋₄₀ alkenyl, and C₃₋₄₀ alkynyl.

In some embodiments, Y² is C₁₋₄ alkylene, W is O or S, and L² is selected from C₃₋₄₀ alkyl, C₃₋₄₀ alkenyl, and C₃₋₄₀ alkynyl.

In some embodiments, Y² is absent, W is absent, O, or S, and L² is selected from C₃₋₄₀ alkyl, C₃₋₄₀ alkenyl, and C₃₋₄₀ alkynyl, optionally W is absent.

In some embodiments, W is O or S. In some embodiments, W is O. In some embodiments, W is absent.

In some embodiments, L² is selected from C₆₋₂₅ alkyl, C₆₋₂₅ alkenyl, and C₆₋₂₅ alkynyl. In some embodiments, L² is selected from C₁₀₋₂₀ alkyl, C₁₀₋₂₀ alkenyl, and C₁₀₋₂₀ alkynyl. In some embodiments, L² is C₁₀₋₂₀ alkyl.

In some embodiments, L² is C₆₋₂₅ alkyl.

In some embodiments, L² is branched. In some embodiments, L² is unbranched.

In some embodiments, Y² is C₁₋₄ alkylene, W is absent, and L² is cholesterol or cholesterol derivative attached to Y² via an O atom of the cholesterol or via an O or S atom of the cholesterol derivative. It is contemplated that the cholesterol derivative can be any suitable cholesterol known in the art with an O atom available for attachment to the rest of the compound of Formula I. For example, a suitable cholesterol derivative can be selected from Beta-sitosterol, stigmasterol, lanosterol, thiocholesterol, stigmastanol, campesterol, fucosterol, brassicasterol and ergosterol

In some embodiments, Y² is C₁₋₂ alkylene, W is absent, and, L² is cholesterol attached to Y² via an O atom of the cholesterol.

### Compound of Formula IA

In some embodiments, the compound of formula I is of Formula IA

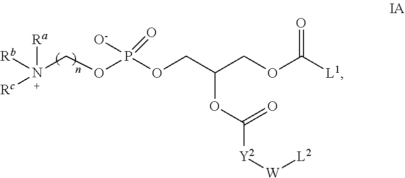

or a pharmaceutically acceptable salt thereof.

In some embodiments, n is 2 to 4. In some embodiments, n is 2.

In some embodiments, Y² is absent and L² is selected from C₃₋₄₀ alkyl, C₃₋₄₀ alkenyl, and C₃₋₄₀ alkynyl.

In some embodiments, L² is C₃₋₄₀ alkyl. In some embodiments, L² is C₆₋₂₅ alkyl. In some embodiments, L² is C₁₀₋₂₀ alkyl.

In some embodiments, W is absent or O. In some embodiments, W is absent. In some embodiment, W is O.

In some embodiments, Y² is C₁₋₄ alkylene and L² is cholesterol or cholesterol derivative attached to Y² via an O atom of the cholesterol or cholesterol derivative. The cholesterol derivative is as defined herein.

In some embodiments, Y² is C₁₋₃ alkylene and L² is cholesterol.

In some embodiments, Y² is an alkylene substituted with one oxo, such as a C₁₋₄ alkylene or C₁₋₃ alkylene substituted with an oxo. In this case, L2 can be attached to Y² via an ester linkage. For example, the cholesterol or the cholesterol derivative can be attached to the rest of the compound of Formula IA via an ester linkage or a thioester linkage with Y².

### Compound of Formula IB and IC

In some embodiments, the compound of formula I is of Formula IB

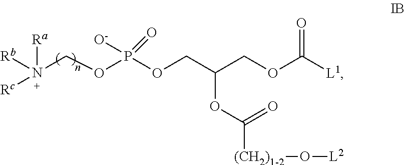

or a pharmaceutically acceptable salt thereof.

In some embodiments, the compound of formula I is of Formula IC

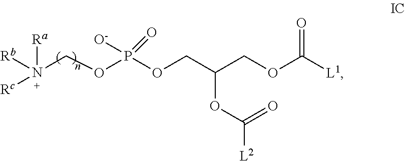

or a pharmaceutically acceptable salt thereof.

In some embodiments, n is 2 to 4. In some embodiments, n is 2.

In some embodiments, L² is selected from C₃₋₄₀ alkyl, C₃₋₄₀ alkenyl, and C₃₋₄₀ alkynyl. In some embodiments, L² is C₆₋₂₅ alkyl. In some embodiments, L² is C₁₀₋₂₀ alkyl. In some embodiments, L² is branched. In some embodiments, L² is unbranched.

### Compound of Formula ID

In some embodiments, the compound of formula I is of Formula ID

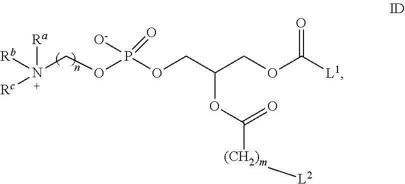

or a pharmaceutically acceptable salt thereof,
wherein m is 1 to 3.

In some embodiments, L² is cholesterol or cholesterol derivative attached to Y² via an O atom of the cholesterol or via an O or S atom of the cholesterol derivative. The cholesterol derivative is as defined herein.

In some embodiments, m is 1 or 2. In some embodiments, m is 1.

For compounds of Formulae IA, to ID, in some embodiments, R^{a}, R^{b}, and R^{c} are each independently C₁₋₃ alkyl. Optionally, R^{a}, R^{b}, and R^{c} are each independently methyl.

In some embodiments, L¹ is C₆₋₂₅ alkyl, C₆₋₂₅ alkenyl, or C₆₋₂₅ alkynyl. In some embodiments, L¹ is C₆₋₂₅ alkyl. In some embodiments, L¹ is C_{10 to 20} alkyl. In some embodiments, L¹ is C₆₋₂₅ alkenyl. In some embodiments, L¹ is C₁₀₋₂₀ alkenyl.

In some embodiments, L¹ is branched. In some embodiments, L¹ is unbranched.

### Exemplary Compounds of Formula I

In some embodiments, the compound of Formula I is selected from the compounds of Table A, the compounds of Table B, and the compounds of Table C, or a pharmaceutically acceptable salt thereof. In some embodiments, the compound of Formula I-I is selected from the compounds of Table A, the compounds of Table B, and the compounds of Table C, or a pharmaceutically acceptable salt thereof.

In some embodiments, the compound of Formula I is selected from the compounds of Table A or a pharmaceutically acceptable salt thereof.

TABLE A
Exemplary Compounds of the Present Disclosure

| | |
|---|---|
| 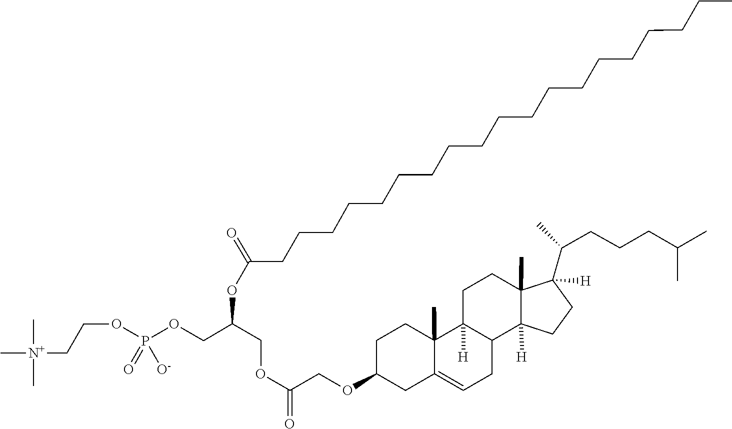 | I-1 |
| 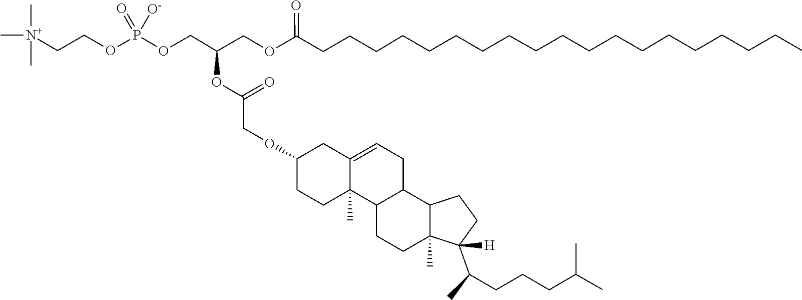 [SMILES: CCCCCCCCCCCCCCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1] | I-2 |
| 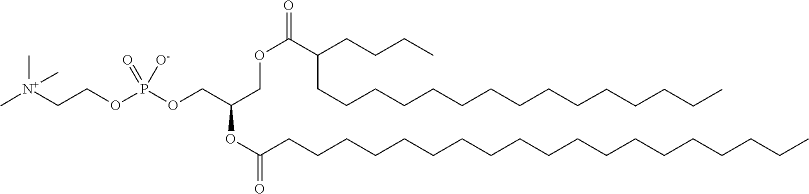 [SMILES: CCCCCCCCCCCCCCCCCCCC(=O)O[C@H](COC(=O)C(CCCC)CCCCCCCCCCCCCCC)COP(=O)([O-])OCC[N+](C)(C)C] | I-3 |
| 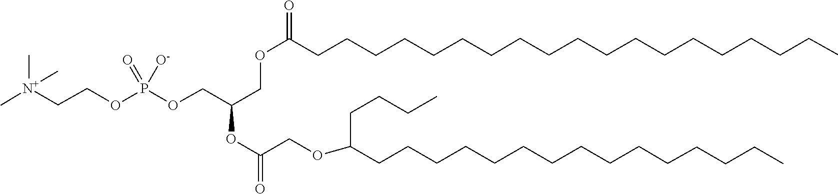 [SMILES: CCCCCCCCCCCCCCCCCCCC(=O)OC[C@H](COP(=O)([O-])OCC[N+](C)(C)C)OC(=O)COC(CCCC)CCCCCCCCCCCCCCC] | I-4 |
| 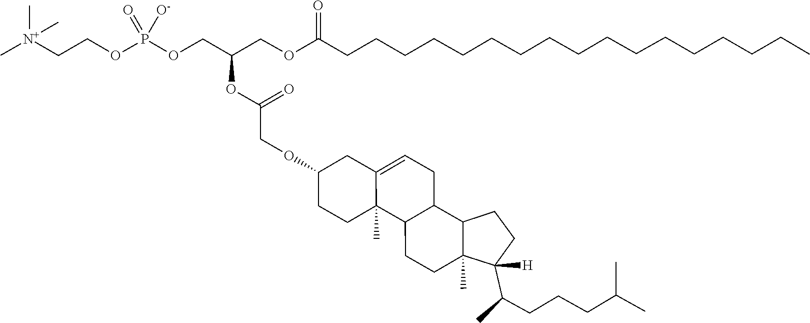 [SMILES: CCCCCCCCCCCCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1] | I-5 |
| 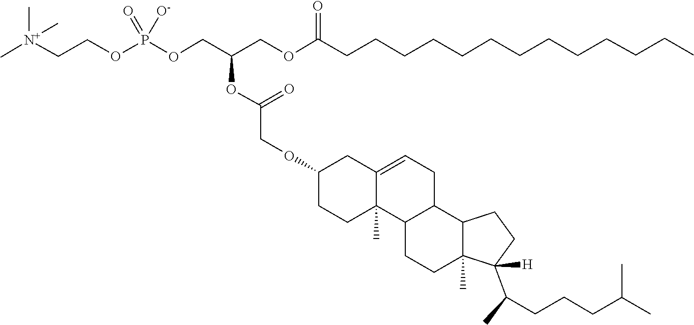 [SMILES: CCCCCCCCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1] | I-6 |
| 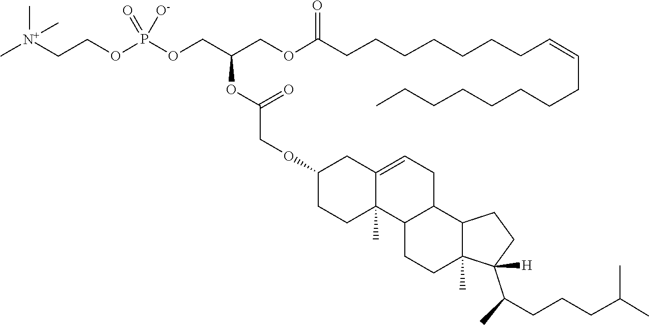 [SMILES: CCCCCCCC/C=C\CCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1] | I-7 |
| 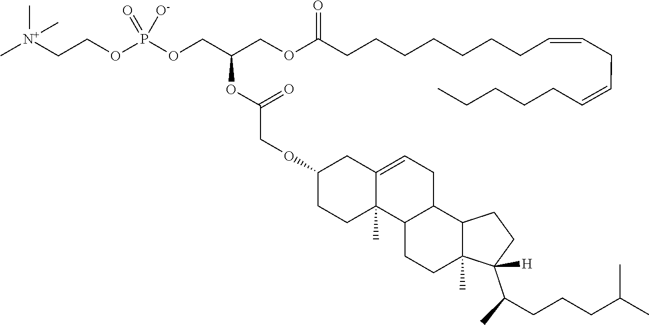 [SMILES: CCCCC/C=C\C/C=C\CCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1] | I-8 |
| 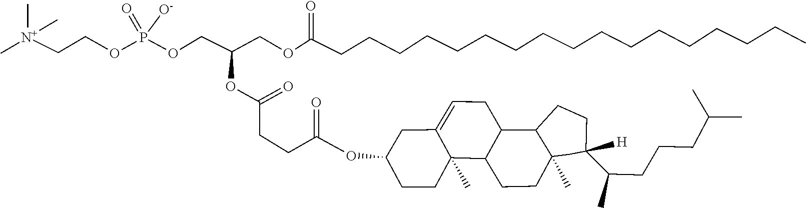 [SMILES: CCCCCCCCCCCCCCCCCC(=O)OC[C@H](COP(=O)([O-])OCC[N+](C)(C)C)OC(=O)CCC(=O)O[C@H]1CC[C@@]2(C)C(=CCC3C4CC[C@H]([C@H](C)CCCC(C)C)C[C@@]4(C)CCC32)C1] | I-9 |
| 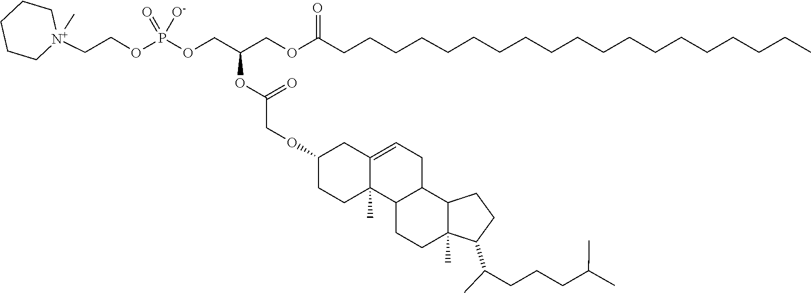 [SMILES: CCCCCCCCCCCCCCCCCCCC(=O)OC[C@H](COP(=O)([O-])OCC[N+]1(C)CCCCC1)OC(=O)CO[C@H]1CC[C@@]2(C)C(=CCC3C2CC[C@@]2(C)C3CC[C@@H]2C(C)CCCC(C)C)C1] | I-10 |
| 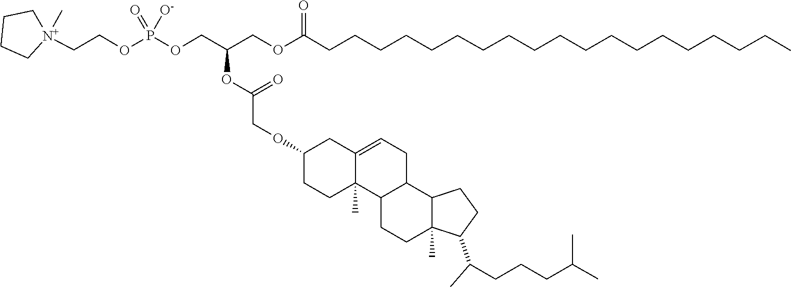 [SMILES: CCCCCCCCCCCCCCCCCCCC(=O)OC[C@H](COP(=O)([O-])OCC[N+]1(C)CCCC1)OC(=O)CO[C@H]1CC[C@@]2(C)C(=CCC3C2CC[C@@]2(C)C3CC[C@@H]2C(C)CCCC(C)C)C1] | I-11 |
| 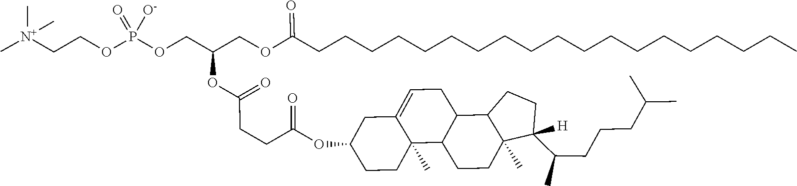 [SMILES: CCCCCCCCCCCCCCCCCCCC(=O)OC[C@H](COP(=O)([O-])OCC[N+](C)(C)C)OC(=O)CCC(=O)O[C@H]1CC[C@@]2(C)C(=CCC3C2CC[C@@]2(C)C3CC[C@@H]2[C@H](C)CCCC(C)C)C1] | I-13 |
| 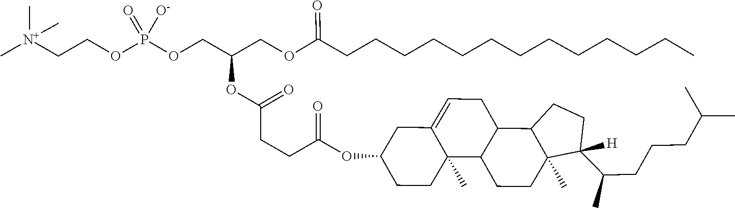 [SMILES: CCCCCCCCCCCCCC(=O)OC[C@H](COP(=O)([O-])OCC[N+](C)(C)C)OC(=O)CCC(=O)O[C@H]1CC[C@@]2(C)C(=CCC3C2CC[C@@]2(C)C3CC[C@@H]2[C@H](C)CCCC(C)C)C1] | I-14 |
| 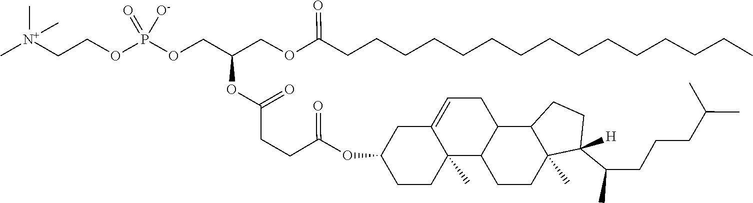 [SMILES: CCCCCCCCCCCCCCCC(=O)OC[C@H](COP(=O)([O-])OCC[N+](C)(C)C)OC(=O)CCC(=O)O[C@H]1CC[C@@]2(C)C(=CCC3C2CC[C@@]2(C)C3CC[C@@H]2[C@H](C)CCCC(C)C)C1] | I-15 |
| 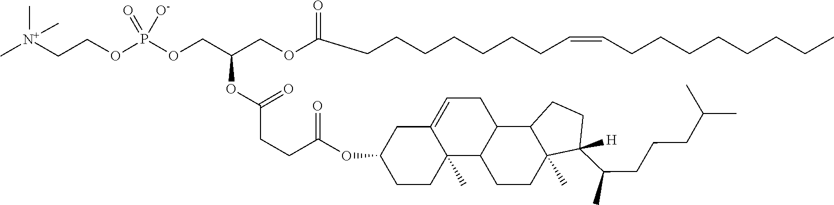 [SMILES: CCCCCCCC/C=C\CCCCCCCC(=O)OC[C@H](COP(=O)([O-])OCC[N+](C)(C)C)OC(=O)CCC(=O)O[C@H]1CC[C@@]2(C)C(=CCC3C2CC[C@@]2(C)C3CC[C@@H]2[C@H](C)CCCC(C)C)C1] | I-16 |
| 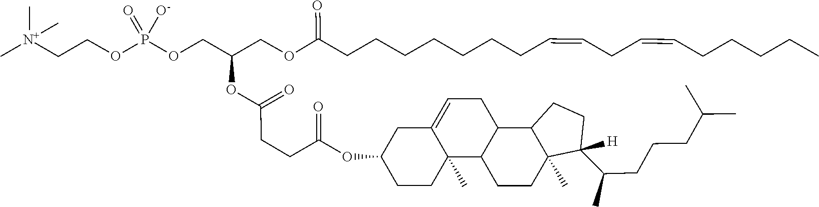 [SMILES: CCCCC/C=C\C/C=C\CCCCCCCC(=O)OC[C@H](COP(=O)([O-])OCC[N+](C)(C)C)OC(=O)CCC(=O)O[C@H]1CC[C@@]2(C)C(=CCC3C2CC[C@@]2(C)C3CC[C@@H]2[C@H](C)CCCC(C)C)C1] | I-17 |

In some embodiments, the compound of Formula I is selected from

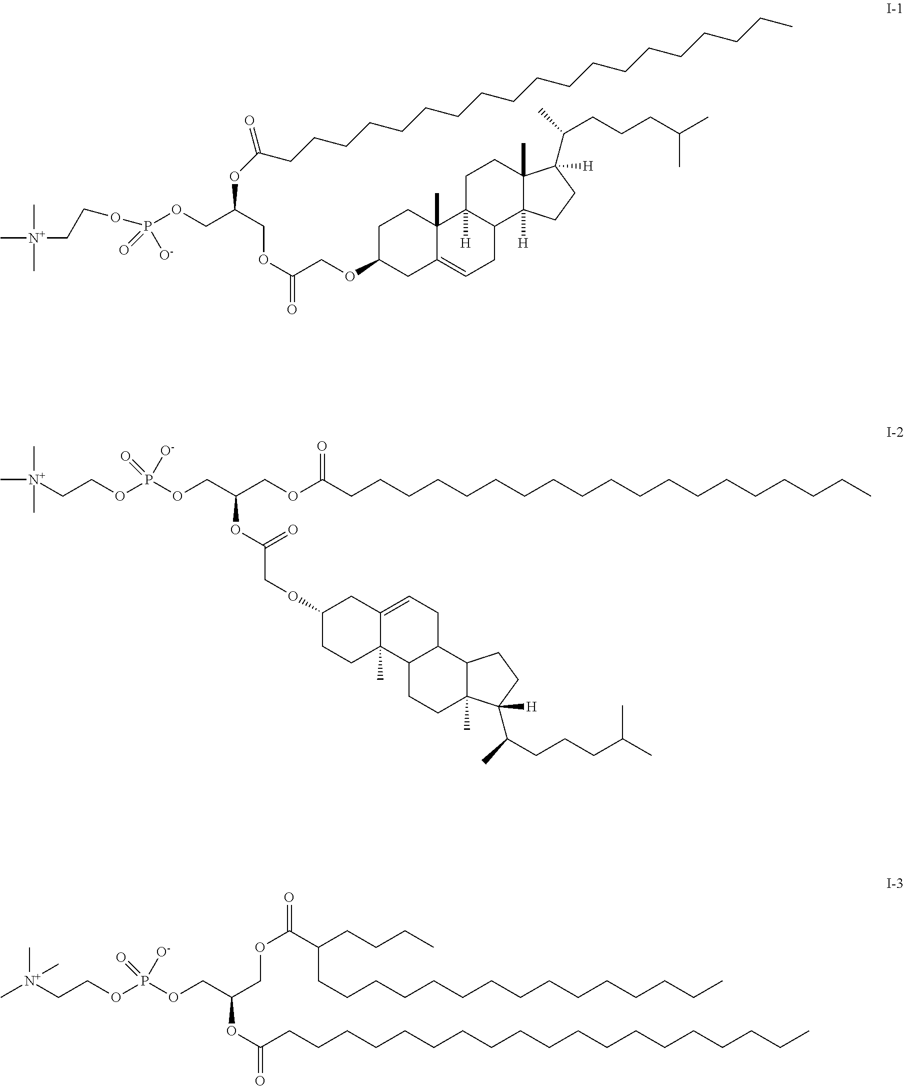

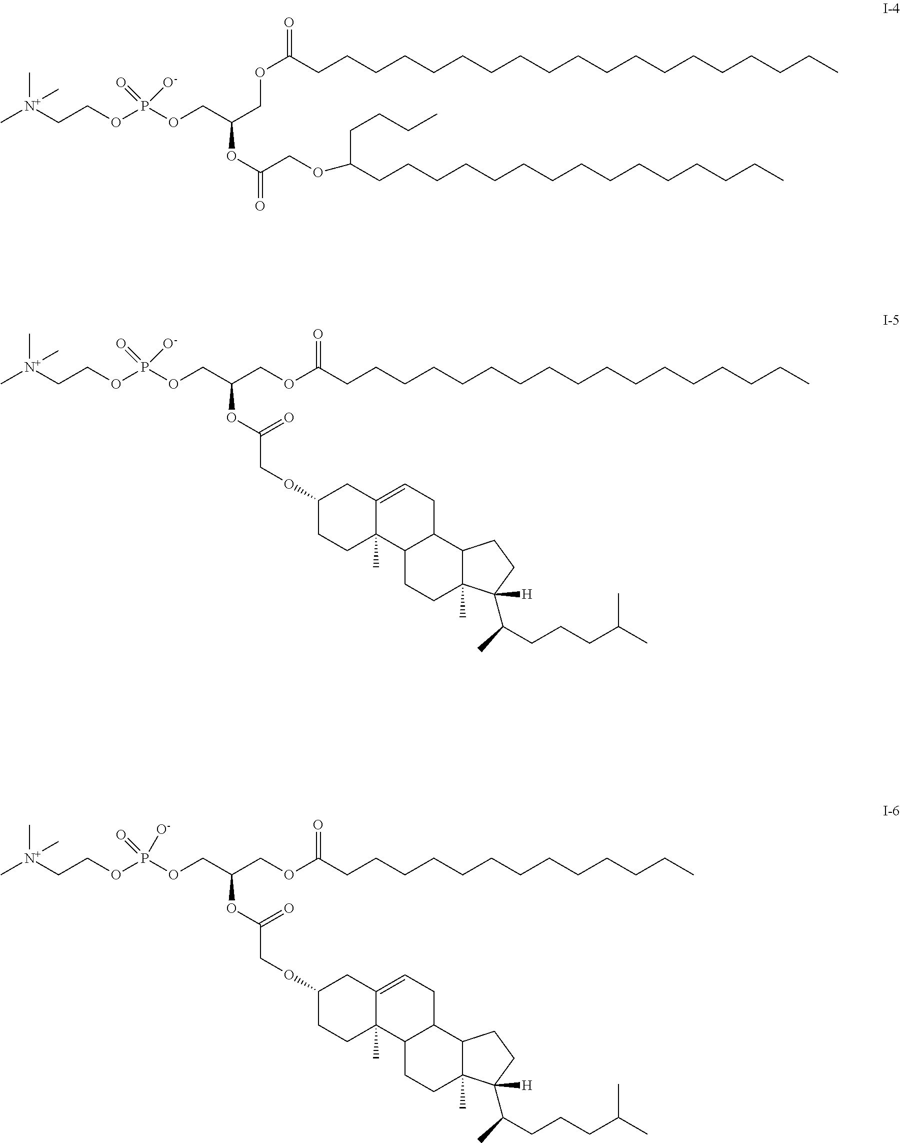

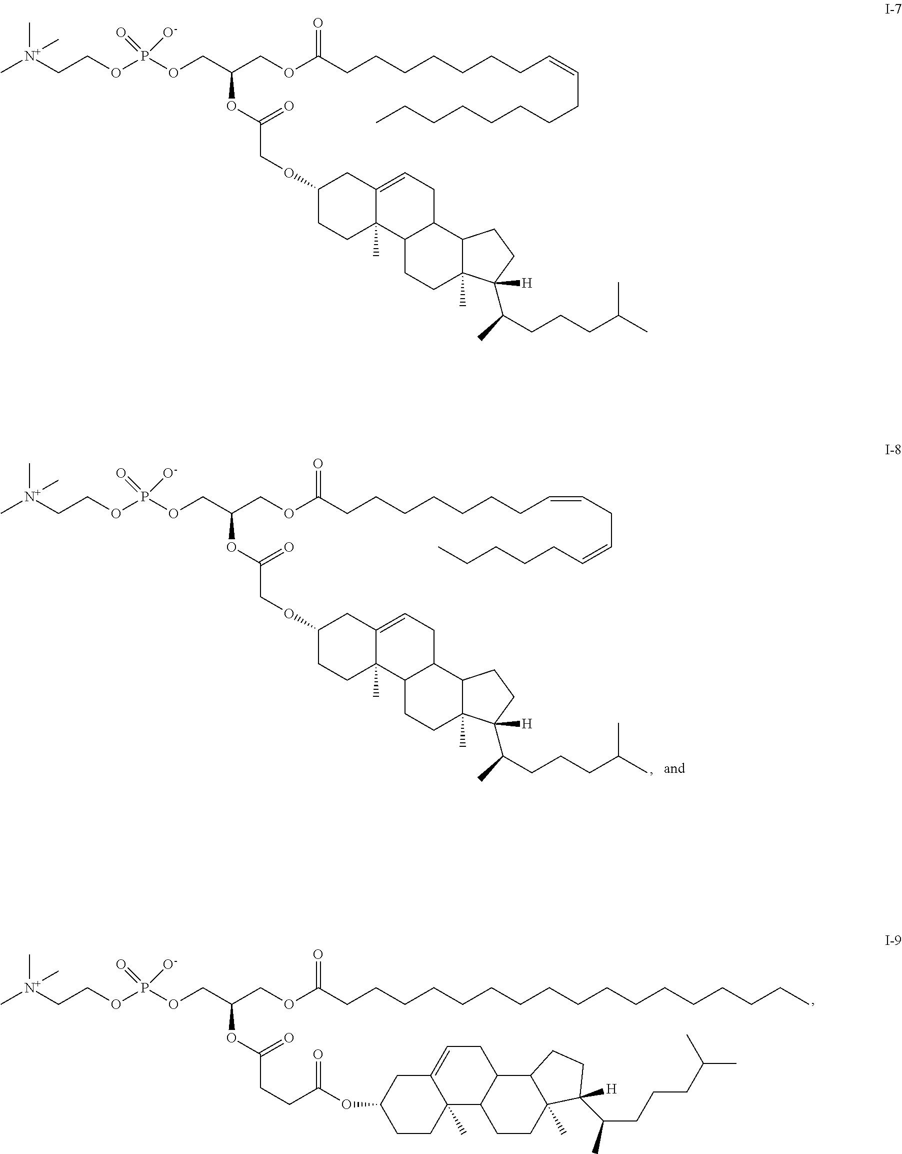

or a pharmaceutically acceptable salt thereof.

In some embodiments, the compound of Formula I is selected from

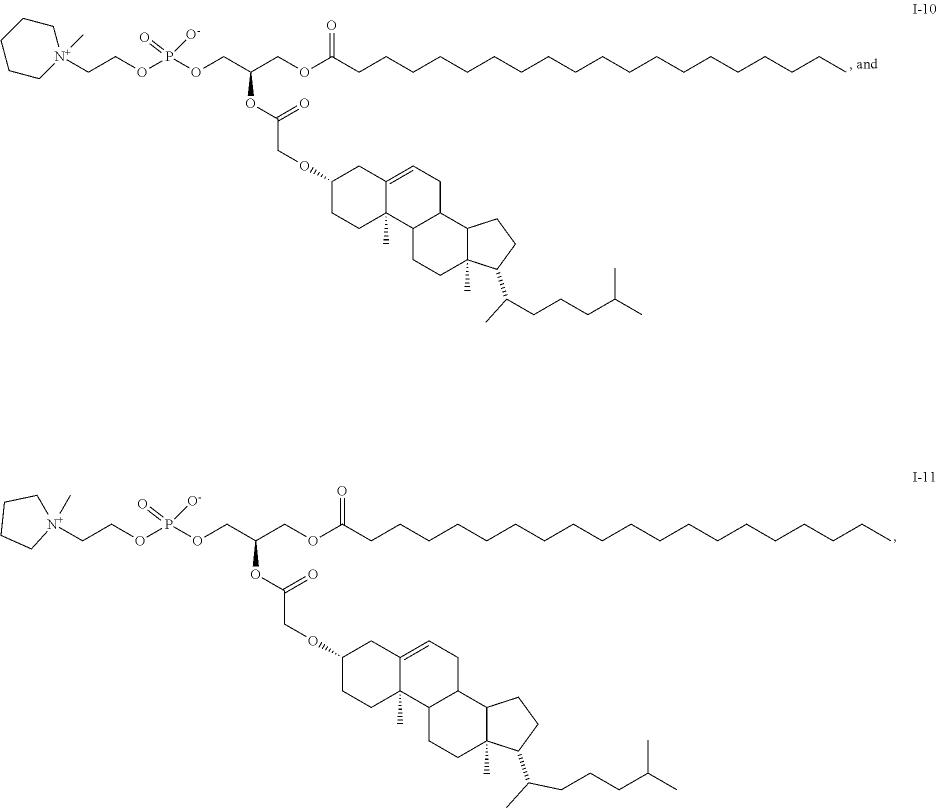 [SMILES: CCCCCCCCCCCCCCCCCCCC(=O)OC[C@H](COP(=O)([O-])OCC[N+]1(C)CCCCC1)OC(=O)CO[C@H]1CC[C@@]2(C)C(=CCC3C2CC[C@@]2(C)C3CC[C@@H]2C(C)CCCC(C)C)C1 ; CCCCCCCCCCCCCCCCCCCC(=O)OC[C@H](COP(=O)([O-])OCC[N+]1(C)CCCC1)OC(=O)CO[C@H]1CC[C@@]2(C)C(=CCC3C2CC[C@@]2(C)C3CC[C@@H]2C(C)CCCC(C)C)C1]

or a pharmaceutically acceptable salt thereof.

In some embodiments, Y² is C₁₋₄ alkylene, W is absent, and L² is cholesterol or cholesterol derivative attached to Y² via an O of the cholesterol or via an O or S atom of the cholesterol derivative. In some embodiments, L² is a cholesterol derivative. For example, the compound of the present disclosure comprising a cholesterol derivative can be selected from the compounds of Table B and Table C below or a pharmaceutically acceptable salt thereof.

TABLE B
Exemplary compounds comprising cholesterol derivatives

| Compound number | R^{D} |
|---|---|
| 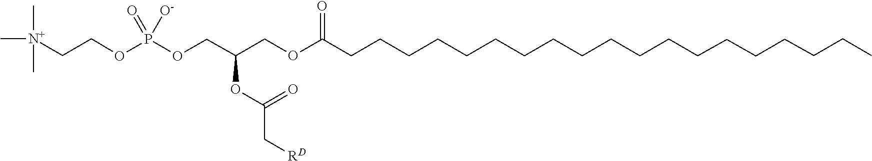 | |
| I-2-1 | 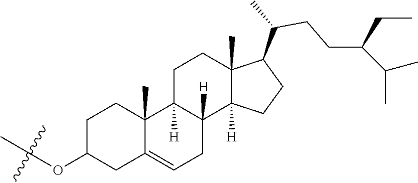 |
| I-2-2 | 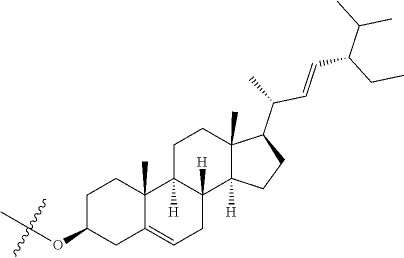 |
| I-2-3 | 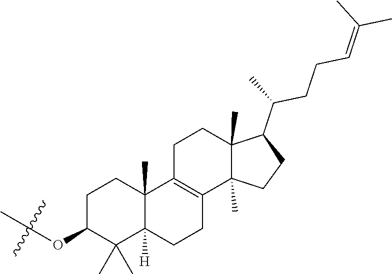 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] |
| I-2-4 | 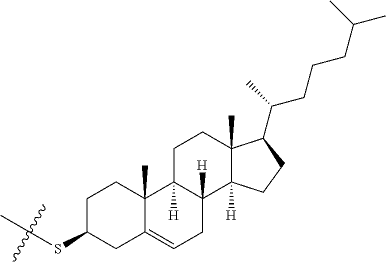 |
| I-2-5 | 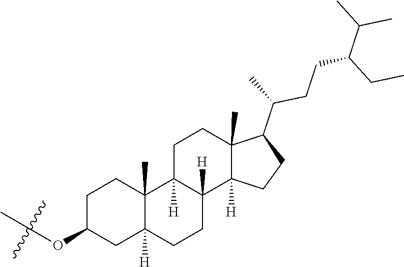 |
| I-2-6 | 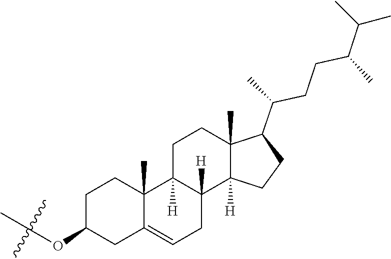 |
| I-2-7 | 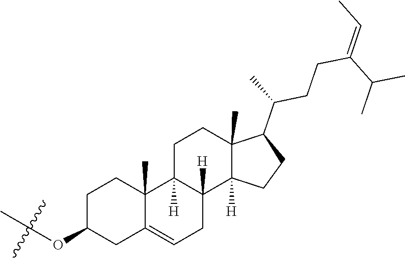 |
| I-2-8 | 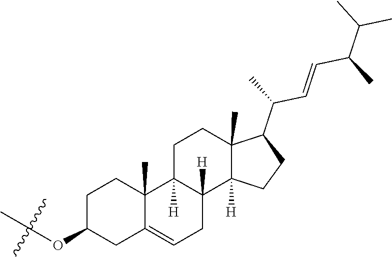 |
| I-2-9 | 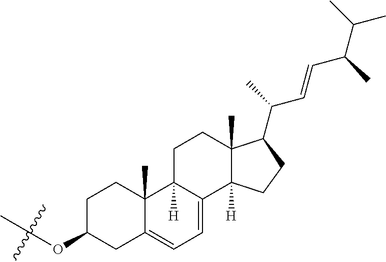 |
| 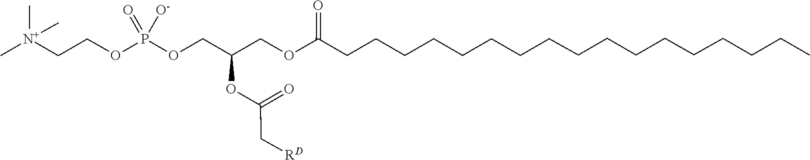 | |
| I-5-1 | 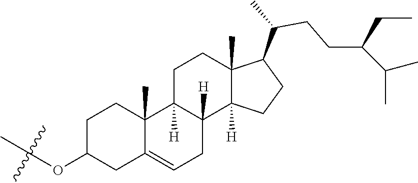 |
| I-5-2 | 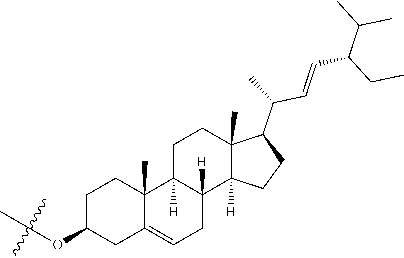 |
| I-5-3 | 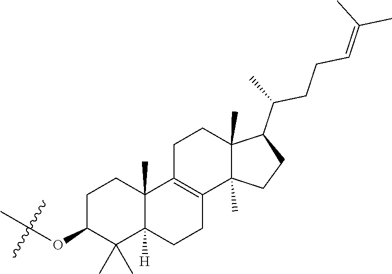 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] |
| I-5-4 | 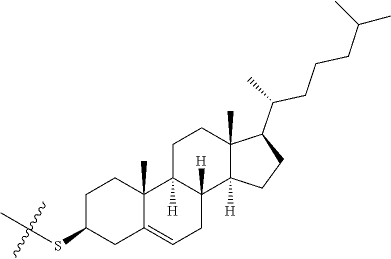 |
| I-5-5 | 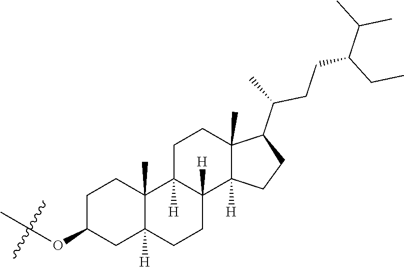 |
| I-5-6 | 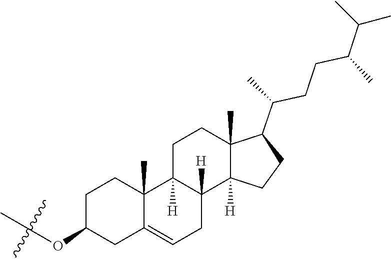 |
| I-5-7 | 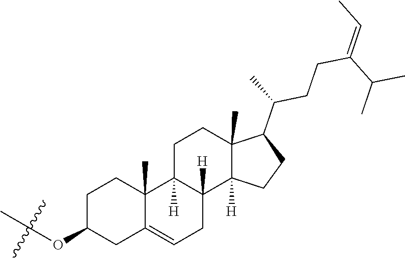 |
| I-5-8 | 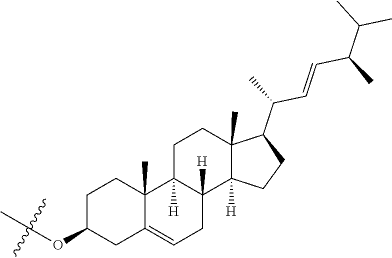 |
| I-5-9 | 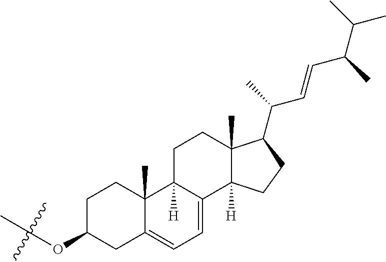 |
| 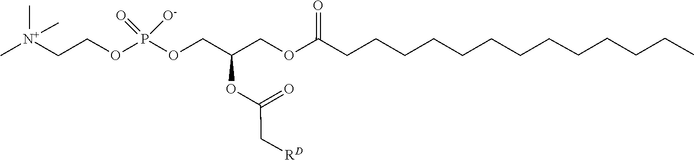 | |
| I-6-1 | 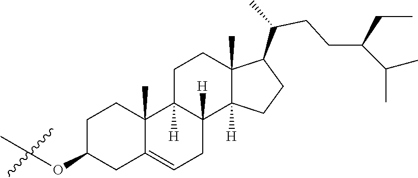 |
| I-6-2 | 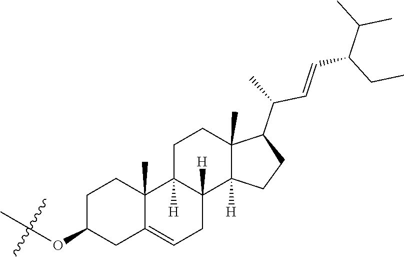 |
| I-6-3 | 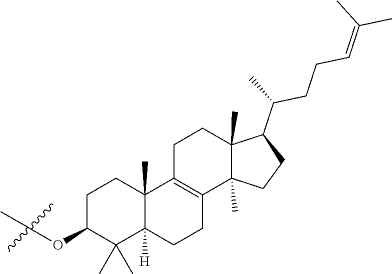 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] |
| I-6-4 | 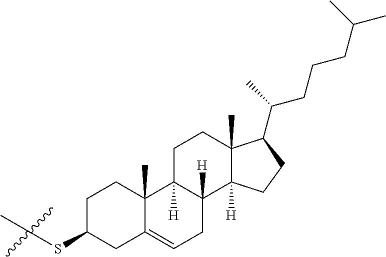 |
| I-6-5 | 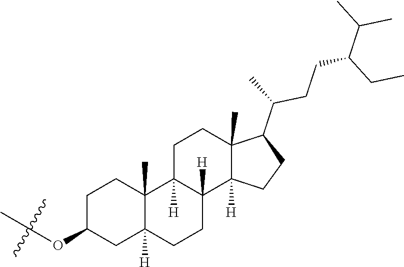 |
| I-6-6 | 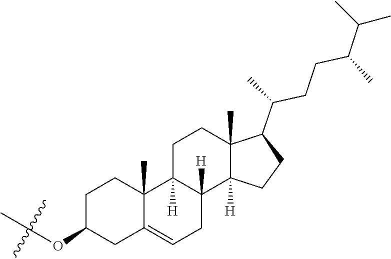 |
| I-6-7 | 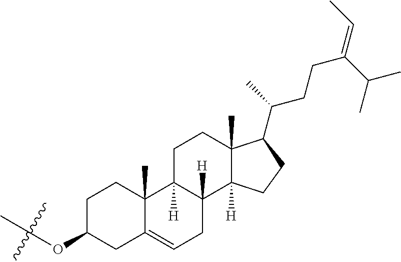 |
| I-6-8 | 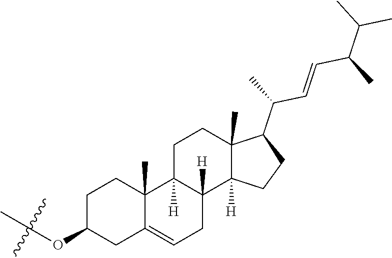 |
| I-6-9 | 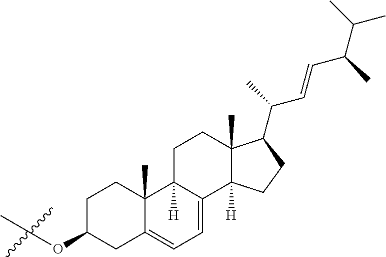 |
| 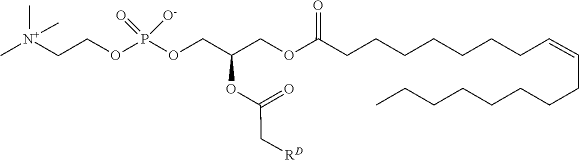 | |
| I-7-1 | 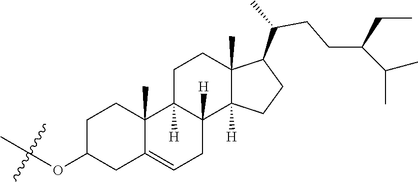 |
| I-7-2 | 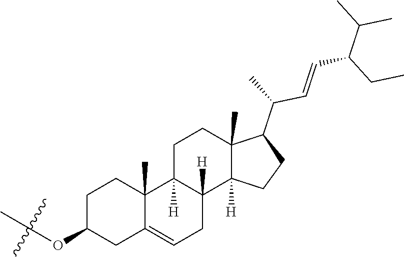 |
| I-7-3 | 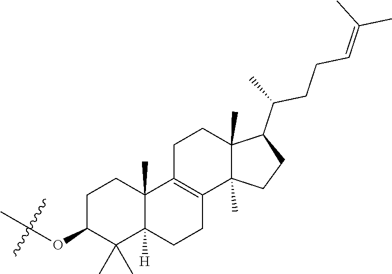 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] |
| I-7-4 | 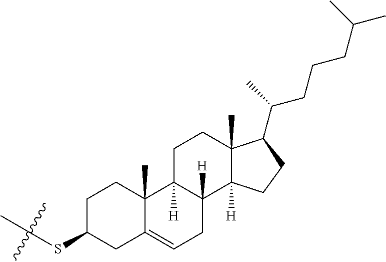 |
| I-7-5 | 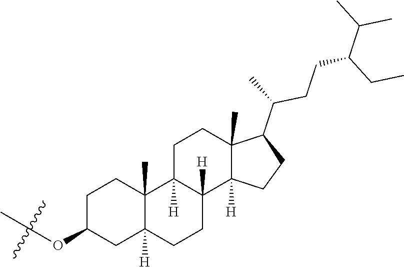 |
| I-7-6 | 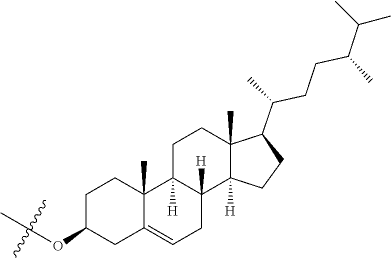 |
| I-7-7 | 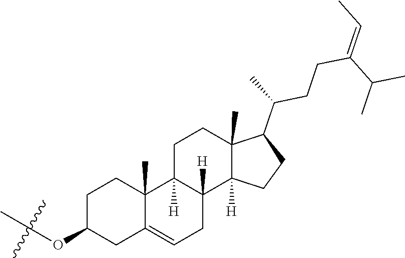 |
| I-7-8 | 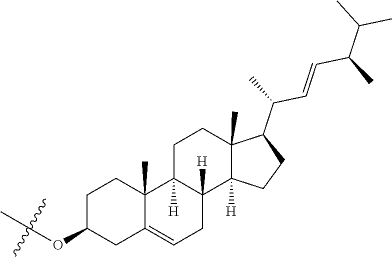 |
| I-7-9 | 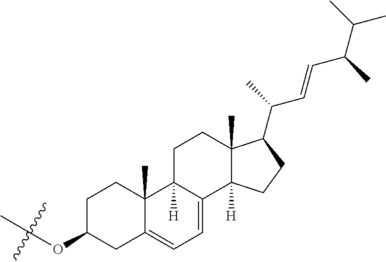 |
| 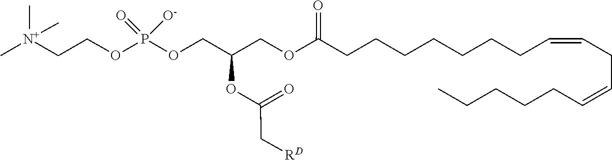 | |
| I-8-1 | 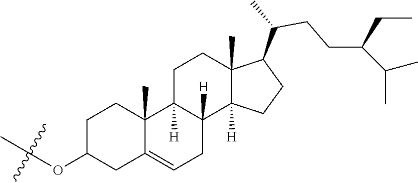 |
| I-8-2 | 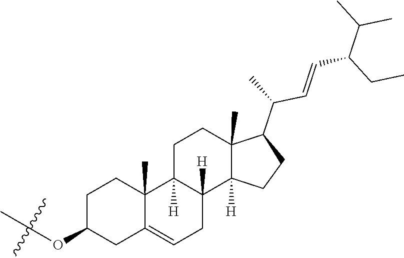 |
| I-8-3 | 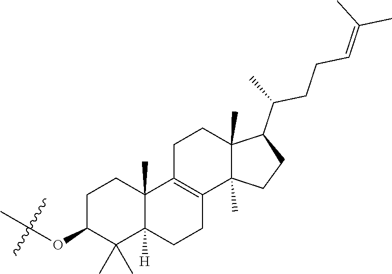 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] |
| I-8-4 | 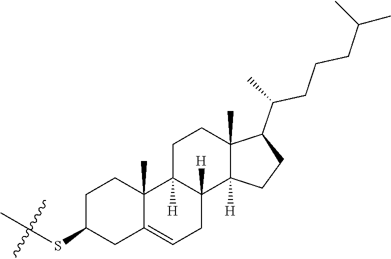 |
| I-8-5 | 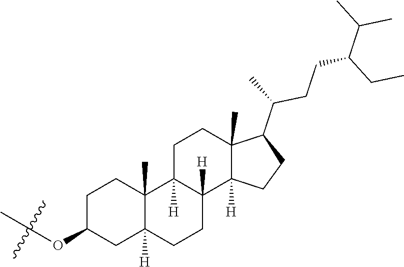 |
| I-8-6 | 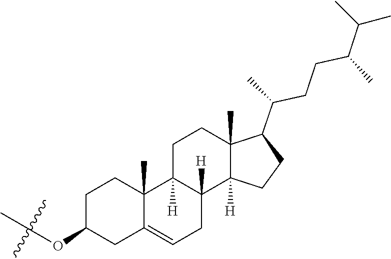 |
| I-8-7 | 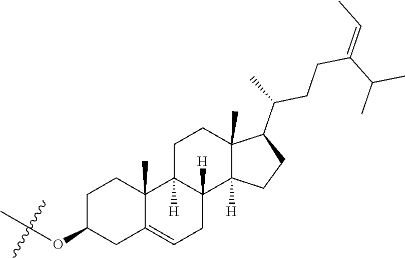 |
| I-8-8 | 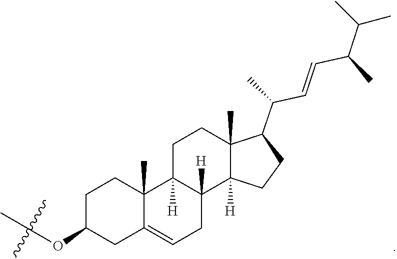 |
| I-8-9 | 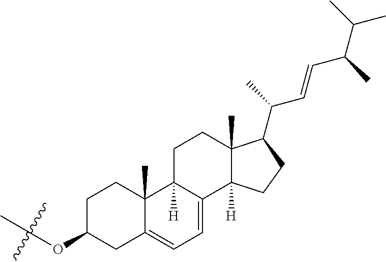 |
| 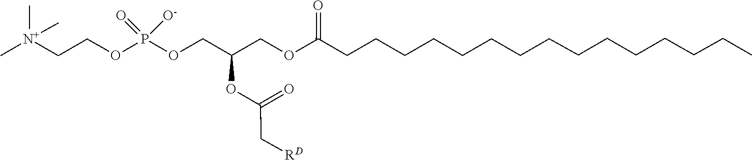 | |
| I-12-1 | 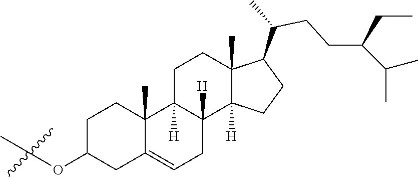 |
| I-12-2 | 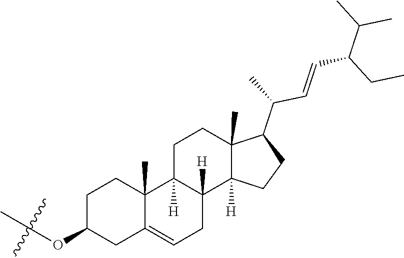 |
| I-12-3 | 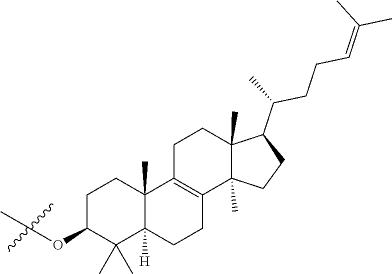 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] |
| I-12-4 | 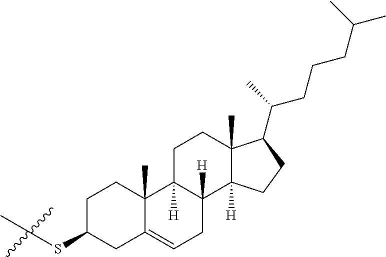 |
| I-12-5 | 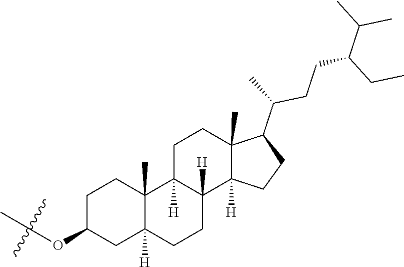 |
| I-12-6 | 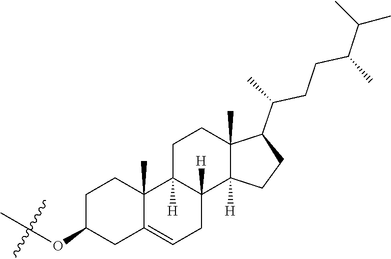 |
| I-12-7 | 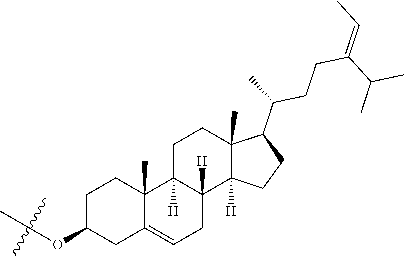 |
| I-12-8 | 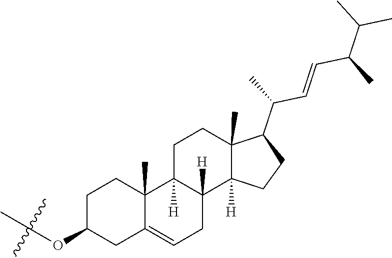 |
| I-12-9 | 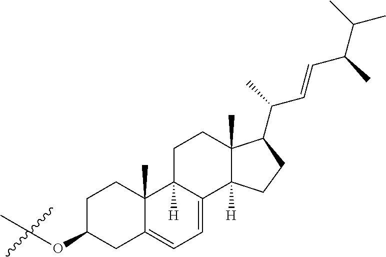 |

TABLE C
Exemplary compounds comprising cholesterol derivatives

| | | |
|---|---|---|
| Compound | | |
| number | R^{D} | R^{E} |

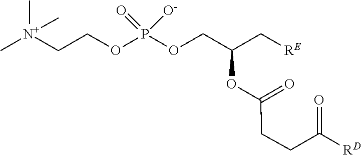

| | | |
|---|---|---|
| I-13-1 | 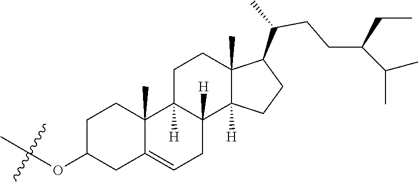 | 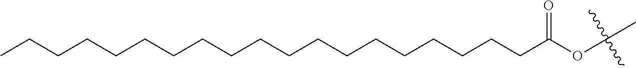 [SMILES: *OC(=O)CCCCCCCCCCCCCCCCCCC] |
| I-13-2 | 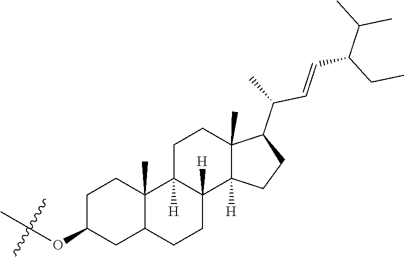 | 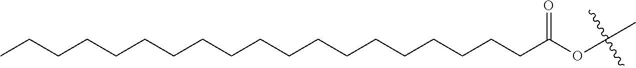 [SMILES: *OC(=O)CCCCCCCCCCCCCCCCCCC] |
| I-13-3 | 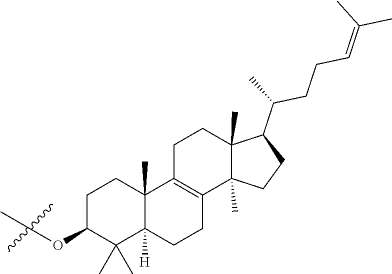 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] | 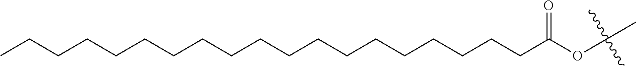 [SMILES: *OC(=O)CCCCCCCCCCCCCCCCCCC] |
| I-13-4 | 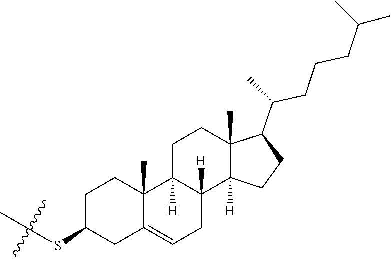 | 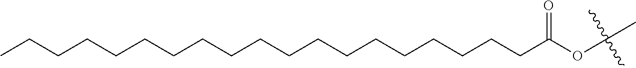 [SMILES: *OC(=O)CCCCCCCCCCCCCCCCCCC] |
| I-13-5 | 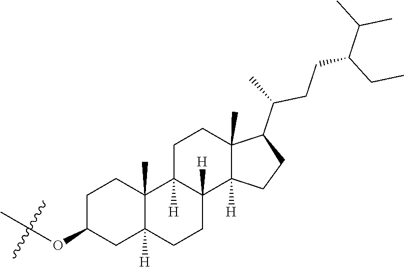 | 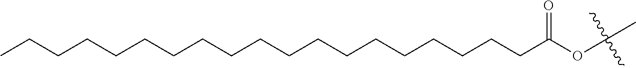 [SMILES: *OC(=O)CCCCCCCCCCCCCCCCCCC] |
| I-13-6 | 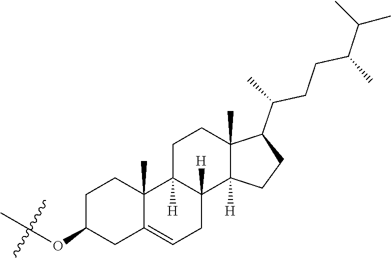 | 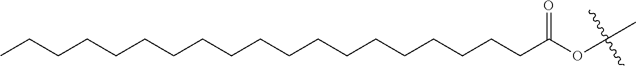 [SMILES: *OC(=O)CCCCCCCCCCCCCCCCCCC] |
| I-13-7 | 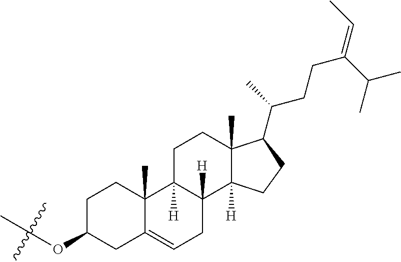 | 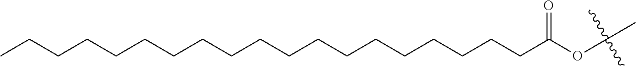 [SMILES: *OC(=O)CCCCCCCCCCCCCCCCCCC] |
| I-13-8 | 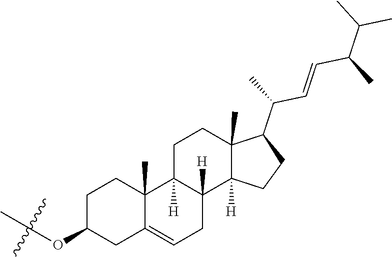 | 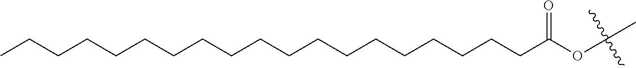 [SMILES: *OC(=O)CCCCCCCCCCCCCCCCCCC] |
| I-13-9 | 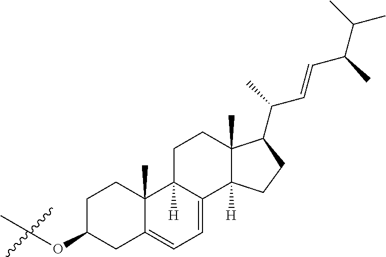 | 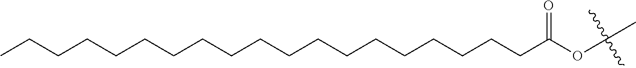 [SMILES: *OC(=O)CCCCCCCCCCCCCCCCCCC] |
| I-9-1 | 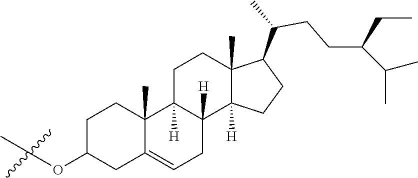 | 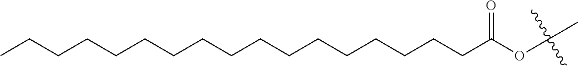 [SMILES: *OC(=O)CCCCCCCCCCCCCCCCC] |
| I-9-2 | 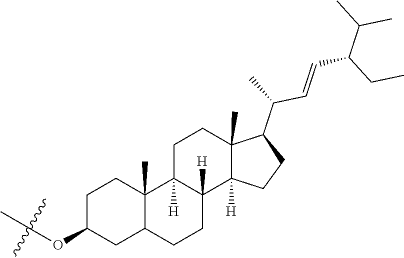 | 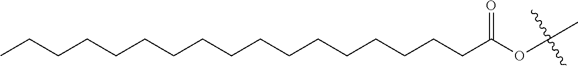 [SMILES: *OC(=O)CCCCCCCCCCCCCCCCC] |
| I-9-3 | 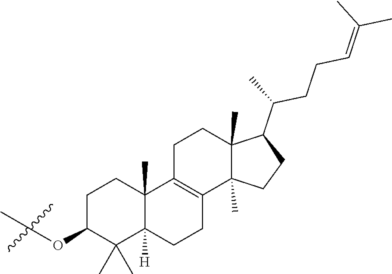 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] | 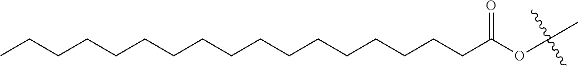 [SMILES: *OC(=O)CCCCCCCCCCCCCCCCC] |
| I-9-4 | 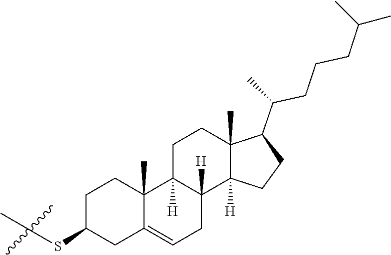 | 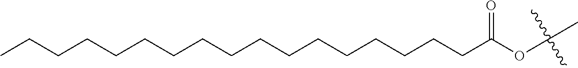 [SMILES: *OC(=O)CCCCCCCCCCCCCCCCC] |
| I-9-5 | 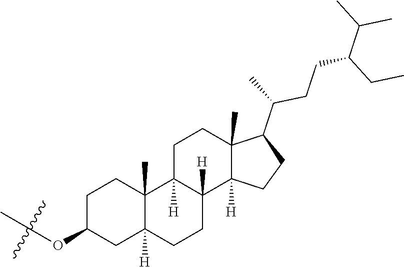 | 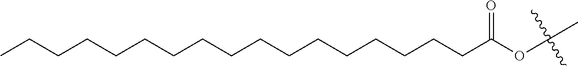 [SMILES: *OC(=O)CCCCCCCCCCCCCCCCC] |
| I-9-6 | 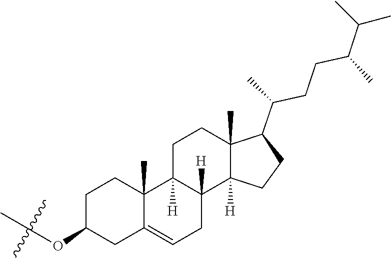 | 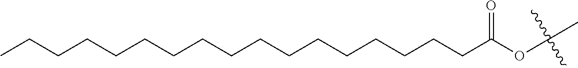 [SMILES: *OC(=O)CCCCCCCCCCCCCCCCC] |
| I-9-7 | 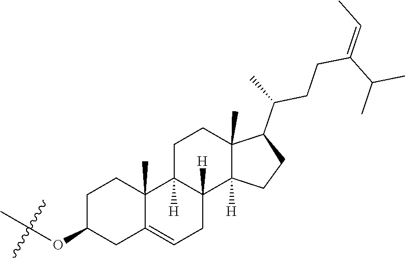 | 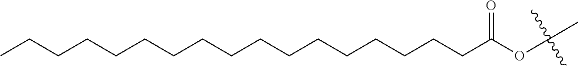 [SMILES: *OC(=O)CCCCCCCCCCCCCCCCC] |
| I-9-8- | 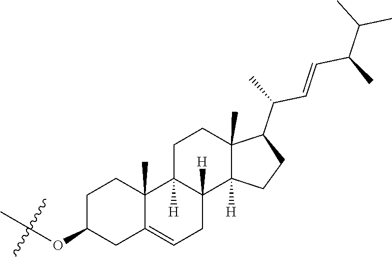 | 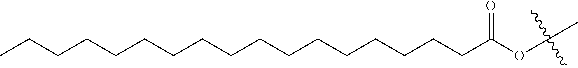 [SMILES: *OC(=O)CCCCCCCCCCCCCCCCC] |
| I-9-9 | 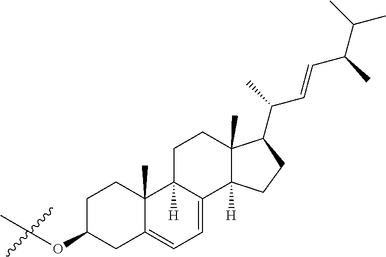 | 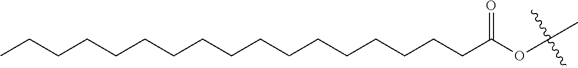 [SMILES: *OC(=O)CCCCCCCCCCCCCCCCC] |
| I-14-1 | 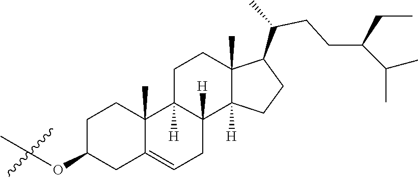 | 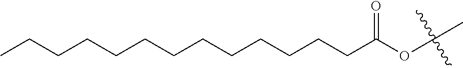 [SMILES: *OC(=O)CCCCCCCCCCCCC] |
| I-14-2 | 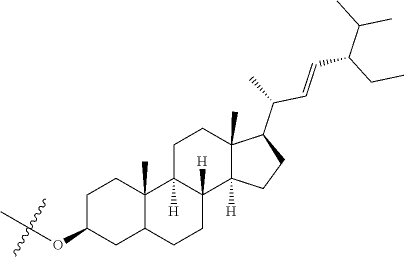 | 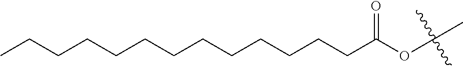 [SMILES: *OC(=O)CCCCCCCCCCCCC] |
| I-14-3 | 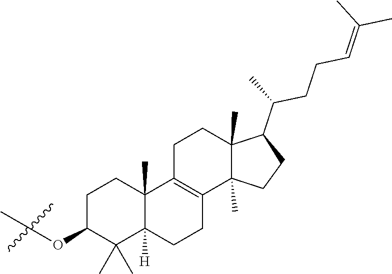 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] | 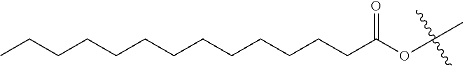 [SMILES: *OC(=O)CCCCCCCCCCCCC] |
| I-14-4 | 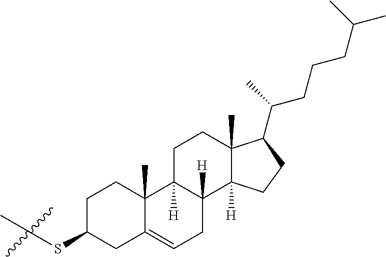 | 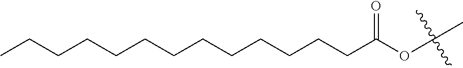 [SMILES: *OC(=O)CCCCCCCCCCCCC] |
| I-14-5 | 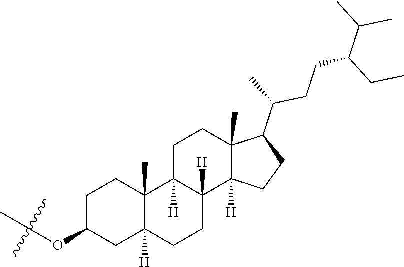 | 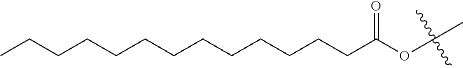 [SMILES: *OC(=O)CCCCCCCCCCCCC] |
| I-14-6 | 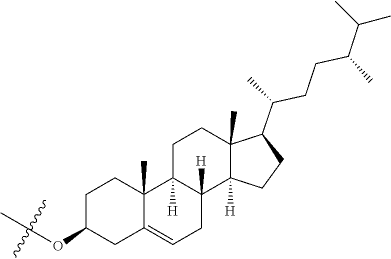 | 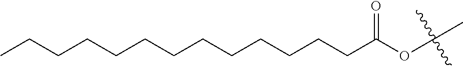 [SMILES: *OC(=O)CCCCCCCCCCCCC] |
| I-14-7 | 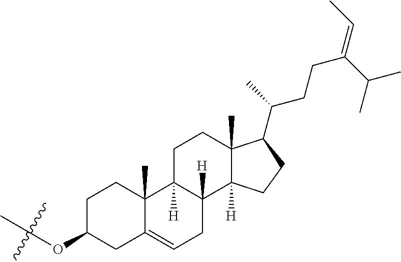 | 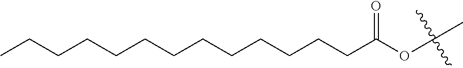 [SMILES: *OC(=O)CCCCCCCCCCCCC] |
| I-14-8 | 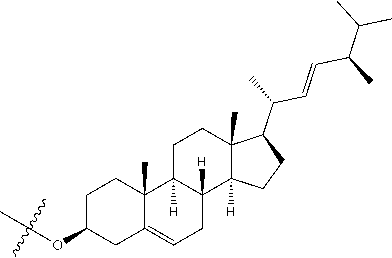 | 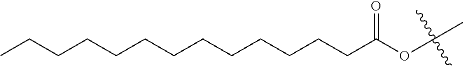 [SMILES: *OC(=O)CCCCCCCCCCCCC] |
| I-14-9 | 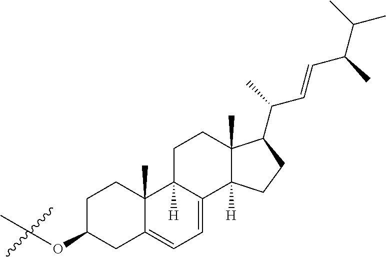 | 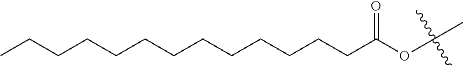 [SMILES: *OC(=O)CCCCCCCCCCCCC] |
| I-15-1 | 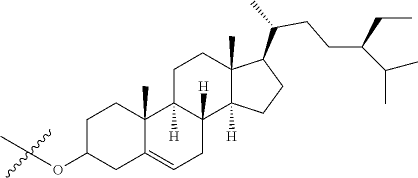 | 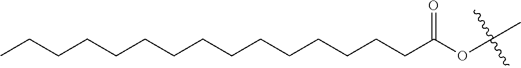 [SMILES: *OC(=O)CCCCCCCCCCCCCCC] |
| I-15-2 | 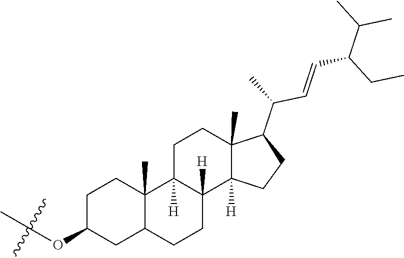 | 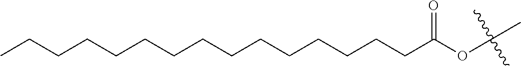 [SMILES: *OC(=O)CCCCCCCCCCCCCCC] |
| I-15-3 | 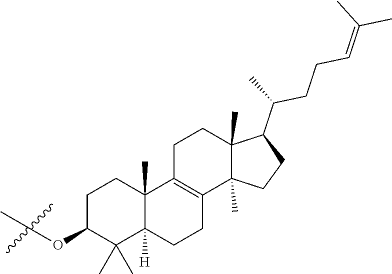 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] | 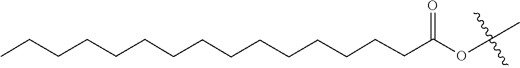 [SMILES: *OC(=O)CCCCCCCCCCCCCCC] |
| I-15-4 | 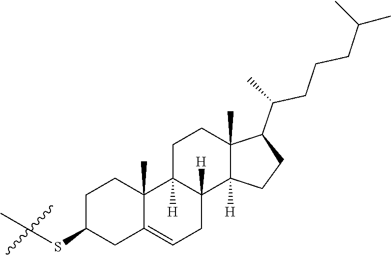 | 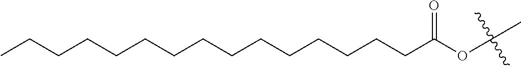 [SMILES: *OC(=O)CCCCCCCCCCCCCCC] |
| I-15-5 | 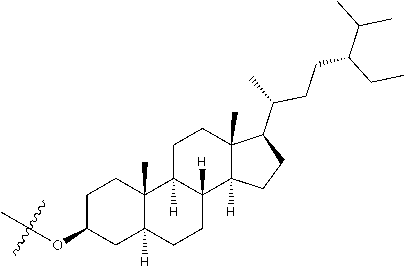 | 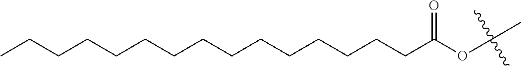 [SMILES: *OC(=O)CCCCCCCCCCCCCCC] |
| I-15-6 | 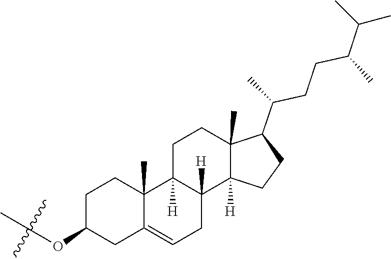 | 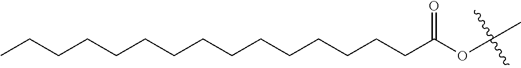 [SMILES: *OC(=O)CCCCCCCCCCCCCCC] |
| I-15-7 | 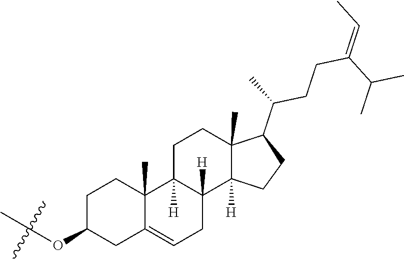 | 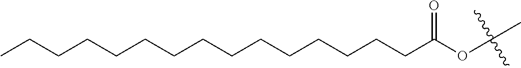 [SMILES: *OC(=O)CCCCCCCCCCCCCCC] |
| I-15-8 | 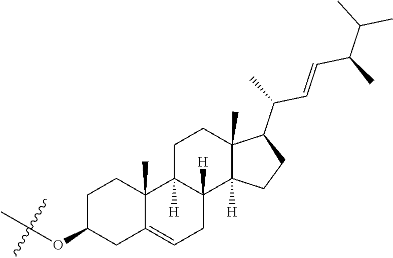 | 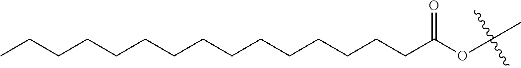 [SMILES: *OC(=O)CCCCCCCCCCCCCCC] |
| I-15-9 | 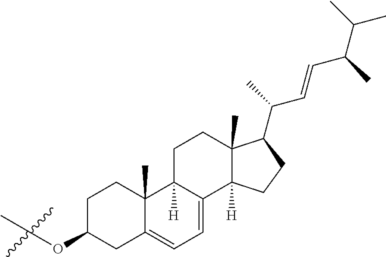 | 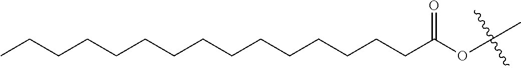 [SMILES: *OC(=O)CCCCCCCCCCCCCCC] |
| I-16-1 | 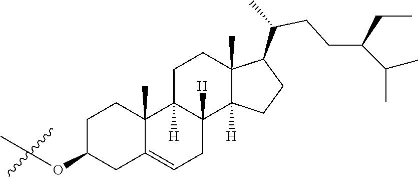 | 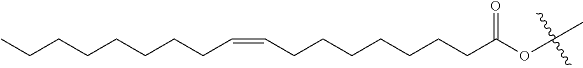 [SMILES: *OC(=O)CCCCCCC/C=C\CCCCCCCC] |
| I-16-2 | 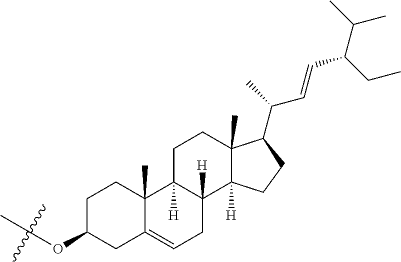 | 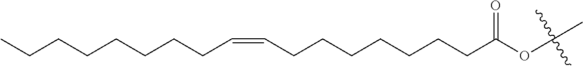 [SMILES: *OC(=O)CCCCCCC/C=C\CCCCCCCC] |
| I-16-3 | 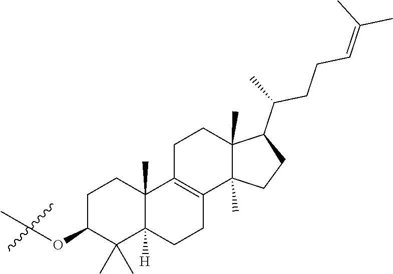 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] | 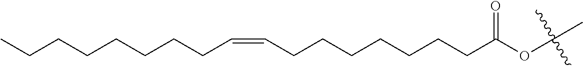 [SMILES: *OC(=O)CCCCCCC/C=C\CCCCCCCC] |
| I-16-4 | 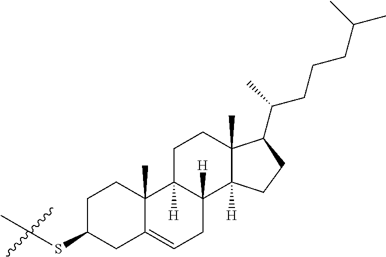 | 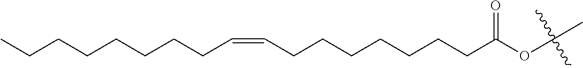 [SMILES: *OC(=O)CCCCCCC/C=C\CCCCCCCC] |
| I-16-5 | 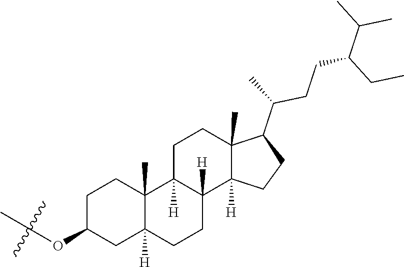 | 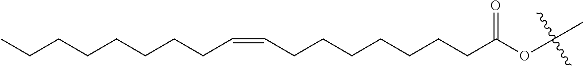 [SMILES: *OC(=O)CCCCCCC/C=C\CCCCCCCC] |
| I-16-6 | 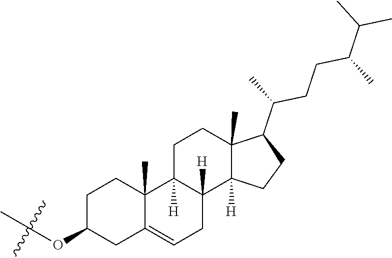 | 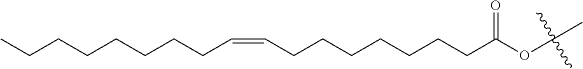 [SMILES: *OC(=O)CCCCCCC/C=C\CCCCCCCC] |
| I-16-7 | 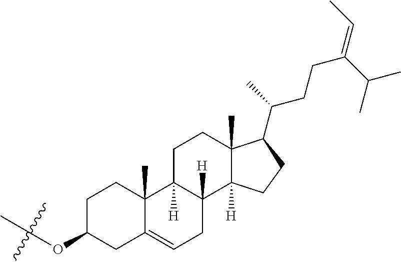 | 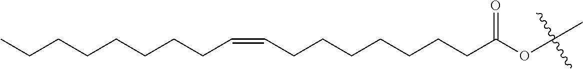 [SMILES: *OC(=O)CCCCCCC/C=C\CCCCCCCC] |
| I-16-8 | 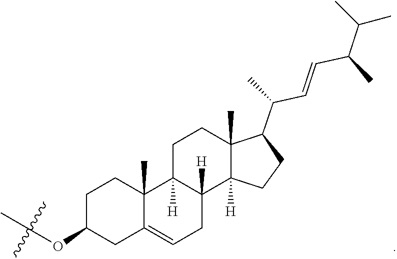 | 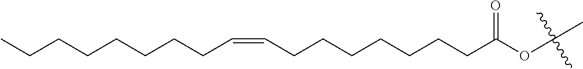 [SMILES: *OC(=O)CCCCCCC/C=C\CCCCCCCC] |
| I-16-9 | 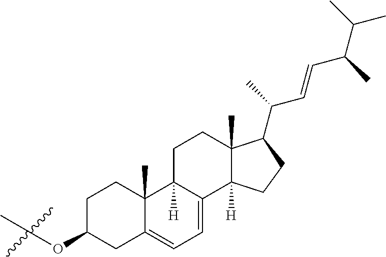 | 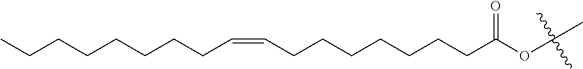 [SMILES: *OC(=O)CCCCCCC/C=C\CCCCCCCC] |
| I-17-1 | 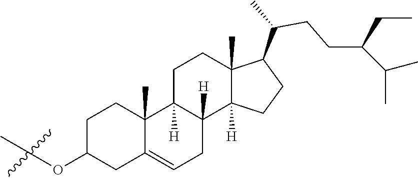 | 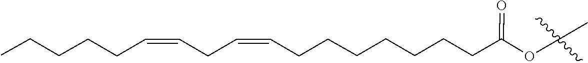 [SMILES: *OC(=O)CCCCCCC/C=C\C/C=C\CCCCC] |
| I-17-2 | 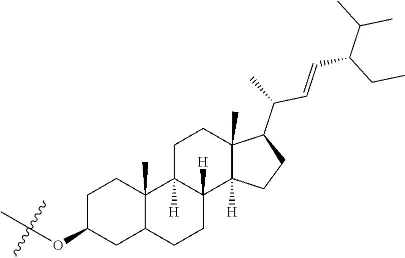 | 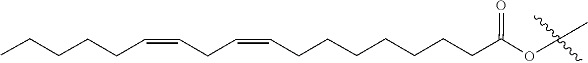 [SMILES: *OC(=O)CCCCCCC/C=C\C/C=C\CCCCC] |
| I-17-3 | 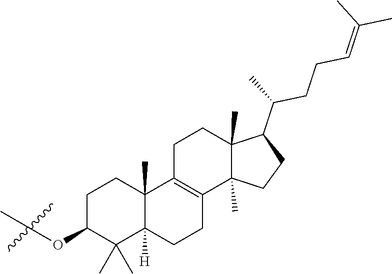 [SMILES: *O[C@H]1CC[C@]2(C)C3=C(CC[C@H]2C1(C)C)[C@]1(C)CC[C@H]([C@H](C)CCC=C(C)C)[C@@]1(C)CC3] | 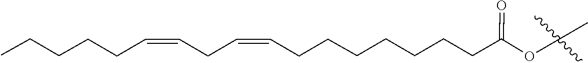 [SMILES: *OC(=O)CCCCCCC/C=C\C/C=C\CCCCC] |
| I-17-4 | 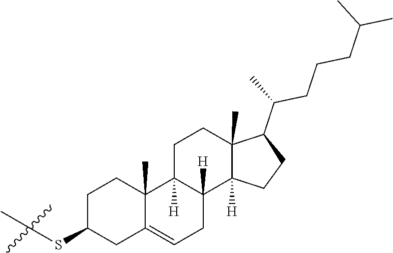 | 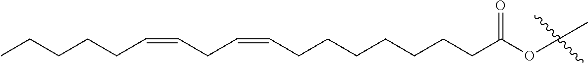 [SMILES: *OC(=O)CCCCCCC/C=C\C/C=C\CCCCC] |
| I-17-5 | 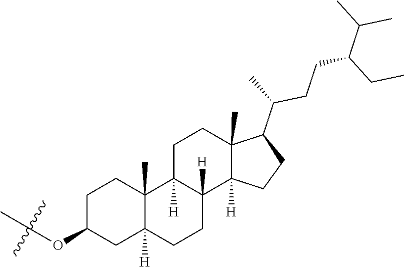 | 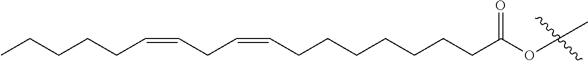 [SMILES: *OC(=O)CCCCCCC/C=C\C/C=C\CCCCC] |
| I-17-6 | 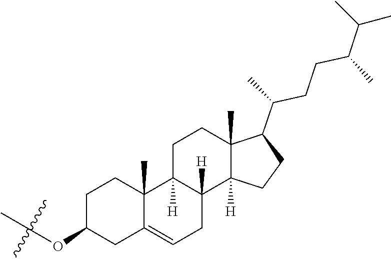 | 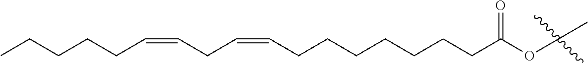 [SMILES: *OC(=O)CCCCCCC/C=C\C/C=C\CCCCC] |
| I-17-7 | 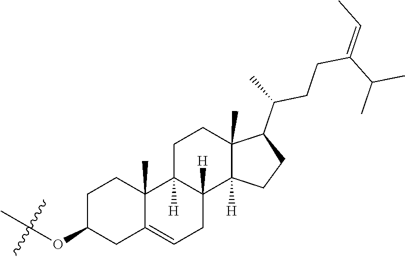 | 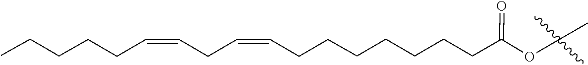 [SMILES: *OC(=O)CCCCCCC/C=C\C/C=C\CCCCC] |
| I-17-8 | 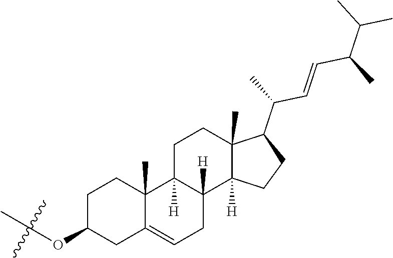 | 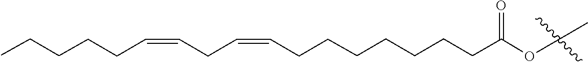 [SMILES: *OC(=O)CCCCCCC/C=C\C/C=C\CCCCC] |
| I-17-9 | 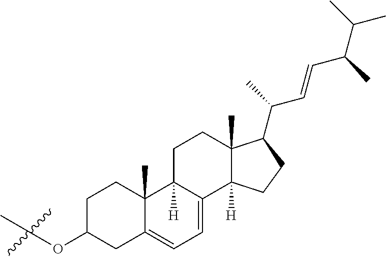 | 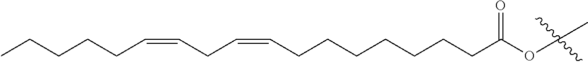 [SMILES: *OC(=O)CCCCCCC/C=C\C/C=C\CCCCC] |

Also falling within the scope herein are the in vivo metabolic products of the compounds described herein. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no activity of their own.

### IV. Pharmaceutical Compositions, Lipid Compositions, Lipid Nanoparticles

Also disclosed herein are pharmaceutical compositions comprising a pharmaceutically effective amount of a compound of the present disclosure (e.g., a compound of Formula I, or a pharmaceutically acceptable salt thereof, and a pharmaceutically acceptable carrier or excipient. Also provided herein is a pharmaceutical composition comprising a pharmaceutically effective amount of a compound of the present disclosure, or a pharmaceutically acceptable salt thereof, and a pharmaceutically acceptable carrier or excipient.

In some embodiments, the present disclosure provides a pharmaceutical composition comprising a compound of the present disclosure or a pharmaceutically acceptable salt or solvate thereof, and a pharmaceutically acceptable excipient.

In some embodiments, the pharmaceutical composition further comprises one or more additional therapeutic agents.

In some embodiments, the present disclosure provides a lipid composition comprising a nucleic acid and a compound of the present disclosure or a pharmaceutically acceptable salt or solvate thereof.

In some embodiments, the nucleic acid is RNA or DNA. In some embodiments, the nucleic acid is selected from an siRNA, an mRNA, a self-replicating RNA, a DNA plasmid, and an antisense oligonucleotide. In some embodiments, the nucleic acid is an siRNA. In some embodiments, the nucleic acid is an mRNA. In some embodiments, the nucleic acid is a self-replicating RNA. In some embodiments, the nucleic acid is a DNA plasmid. In some embodiments, the nucleic acid is an antisense oligonucleotide. In some embodiment, the nucleic acid is a mRNA or a self-replicating RNA comprising a coding region that encodes a protein. In some embodiments, the protein is an enzyme, an antibody, an antigen, a receptor, or a transporter. In some embodiments, the protein is a gene-editing enzyme. In some embodiments, the protein is an enzyme. In some embodiments, the protein is an antibody. In some embodiments, the protein is an antigen. In some embodiments, the protein is a receptor. In some embodiments, the protein is a transporter. For example, the gene-editing enzyme is selected from a TALEN, a CRISPR, a meganuclease, or a zinc finger nuclease.

The nucleic acid may be encapsulated or complexed within the lipid portion of the composition, thereby protecting the nucleic acid from nuclease degradation. In some embodiments, the nucleic acid is substantially fully encapsulated within the lipid portion of the lipid formulation, thereby protecting the nucleic acid from nuclease degradation. In certain instances, the nucleic acid in the lipid formulation is not substantially degraded after exposure of the particle to a nuclease at 37° C. for at least 20, 30, 45, or 60 minutes. In certain other instances, the nucleic acid in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the nucleic acid is complexed with the lipid portion of the formulation.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I0−I)/I0, where I and I0 refer to the fluorescence intensities before and after the addition of detergent.

In some embodiments, the lipid composition (e.g. lipid nanoparticles) comprise a nucleic acid that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the nucleic acid encapsulated therein. The amount may be any value or subvalue within the recited ranges, including endpoints.

It can be appreciated that the lipid composition can exist in different structural forms. For example, the lipid composition comprises liposomes, lipoplexes, or lipid nanoparticles. In some embodiments, the lipid composition comprises or is lipid nanoparticles.

In some embodiments, the present disclosure provides a pharmaceutical composition comprising the lipid nanoparticle of the present disclosure, and a pharmaceutically acceptable excipient.

In some embodiments, the pharmaceutical composition is a lyophilized composition.

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida M Pharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), and release at the cytoplasm (for RNA). Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still undergoing development. The use of mRNA in lipid delivery vehicles quickly rose to prominence as a result of the COVID-19 pandemic with several vaccines delivering mRNA encoding the spike protein of COVID-19 showing strong protective capabilities. Such lipid-based mRNA vaccines include Pfizer and BioNtech's BNT162b2 and Moderna's mRNA-1273, which have received emergency use authorization around the world.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and are within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicles (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int. J. Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid is contained within the liposomal compartment in an aqueous phase.

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed herein below.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell can be formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following IV administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the LIPOFECTAMINE® reagent, have found considerable utility for in vitro transfection. However, these first-generation lipoplexes have not proven useful in vivo. The large particle size and positive charge (imparted by the cationic lipid) result in rapid plasma clearance, hemolytic and other toxicities, as well as immune system activation.

In some embodiments, the present disclosure provides a lipid nanoparticle comprising a plurality of lipids, wherein each lipid is independently a compound of the present disclosure or a pharmaceutically acceptable salt or solvate thereof. For example, the plurality of lipids can self-assemble to form the lipid nanoparticle. In another aspect, the present disclosure provides a lipid nanoparticle comprising a compound of Formula I of the present disclosure or a pharmaceutically acceptable salt or solvate thereof. For example, the compound of the present disclosure or the salt thereof self-assembles into the lipid nanoparticle. It is understood that a lipid nanoparticle generally comprising an interior and exterior.

The lipid nanoparticle can be a range of sizes as measured by for example average diameter. For example, the average particle size of the lipid nanoparticle is less than about 1000 nm, less than about 500 nm, less than about 300 nm, less than about 200 nm, less than about 100 nm, or less than about 90 nm. For example, the average particle size of the lipid nanoparticle is more than about 10 nm, more than about 20 nm, more than about 30 nm, more than about 40 nm, more than about 50 nm, more than about 60 nm, more than about 80 nm, more than about 100 nm, more than about 150 nm, or more than about 200 nm. In some embodiments, the average particle size of the lipid nanoparticle is less than about 100 nm. In some embodiments, the average particle size of the lipid nanoparticle is about 40 nm to about 100 nm, about 50 nm to about 100 nm, about 60 nm to about 100 nm, about 50 nm to about 95 nm, about 50 nm to about 90 nm, or about 60 nm to about 90 nm. The diameter may be any value or subvalue within the recited ranges, including endpoints.

In some embodiments, the lipid nanoparticle has a polydispersity index (PDI) of about 0.010 to about 1.10. In embodiments, the PDI is about 0.010 to about 1.05, about 0.010 to about 1.00, about 0.010 to about 0.95, about 0.010 to about 0.90, about 0.010 to about 0.85, about 0.010 to about 0.80, about 0.010 to about 0.75, about 0.010 to about 0.70, about 0.010 to about 0.65, about 0.010 to about 0.60, about 0.010 to about 0.55, about 0.010 to about 0.50, about 0.010 to about 0.45, about 0.010 to about 0.40, about 0.010 to about 0.35, about 0.010 to about 0.30, about 0.010 to about 0.25, about 0.010 to about 0.20, about 0.010 to about 0.15, about 0.010 to about 0.10, about 0.010 to about 0.09, about 0.010 to about 0.08, about 0.010 to about 0.07, about 0.010 to about 0.06, about 0.010 to about 0.05, about 0.010 to about 0.04, about 0.010 to about 0.03, about 0.010 to about 0.02, about 0.010 to about 0.019, about 0.010 to about 0.018, about 0.010 to about 0.017, about 0.010 to about 0.016, about 0.010 to about 0.015, about 0.010 to about 0.014, about 0.010 to about 0.013, about 0.010 to about 0.012, about 0.010 to about 0.011 (or any ranges therein.) The amount may be any value or subvalue within the recited ranges, including endpoints.

In some embodiments, the lipid nanoparticle is substantially non-toxic. In addition, nucleic acids, when present in the lipid nanoparticles of the present disclosure, are resistant in aqueous solution to degradation with a nuclease.

In some embodiments, the lipid nanoparticle further comprises a nucleic acid. For example, the nucleic acid can be encapsulated in the interior. In some embodiments, the nucleic acid is as defined herein.

### Additional Components

It is contemplated that the pharmaceutical composition, lipid composition and lipid nanoparticle of the present disclosure can further comprise other components. Exemplary additional components can be one or more of other lipids, cholesterol, polyethylene glycol (PEG)-lipid conjugate as described herein. The other lipids can be helper lipids and ionizable cationic lipids as described herein. It is also contemplated that the pharmaceutical composition, lipid composition and lipid nanoparticle of the present disclosure can further comprise other therapeutic agents.

In some embodiments, the lipid nanoparticle further comprises a helper lipid, which can be referred to as a neutral lipid, a neutral helper lipid, non-cationic lipid, non-cationic helper lipid, anionic lipid, anionic helper lipid, or a zwitterionic lipid. In some situations, lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoyl-sn-glycero-3-phosphatidylcholine (DOPC), Di-Oleoyl-Phosphatidyl-Ethanolamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity. Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

In some embodiments, the helper lipid is selected from: dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), and phosphatidylcholine (PC). In some embodiments, the helper lipid is distearoylphosphatidylcholine (DSPC).

Additional examples of non-cationic lipids include sterols such as cholesterol and derivatives thereof. One study found that cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the helper lipid present in the lipid formulation comprises or consists of one or more phospholipids, e.g., a cholesterol-free lipid formulation. In yet other embodiments, the helper lipid present in the lipid formulation comprises or consists of cholesterol or a derivative thereof, e.g., a phospholipid-free lipid formulation. In some embodiments, the lipid composition or nanoparticle further comprises cholesterol.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the lipid nanoparticle further comprises cholesterol. In some embodiments, the lipid nanoparticle is DSPC free. In some embodiments, the lipid nanoparticle is free of helper lipid.

In some embodiments, the lipid nanoparticle further comprises a PEG-lipid conjugate. In some embodiments, the PEG-lipid conjugate is PEG-DMG. Optionally, the PEG-lipid conjugate is PEG2000-DMG.

In some embodiments, the lipid nanoparticle further comprises an ionizable cationic lipid. In some embodiments, the lipid nanoparticle is free from ionizable cationic lipids.

The lipid composition further includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes can be used in non-viral delivery systems for oligonucleotides, including plasmid DNA, antisense oligos, and siRNA/small hairpin RNA-shRNA. Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may include, for example, N,N-dimethyl-N,N-di-9-cis-octadecenylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Chol), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Exemplary suitable cationic lipids are disclosed in International Publication Nos. WO 2023/086514 and WO 2024/233750; and U.S. Pat. Nos. 10,383,952, 10,980,895, 10,526,284, and 10,961,188, the contents of each of which are incorporated by reference herein. In some embodiments, cationic lipids included in lipid formulations provided herein are ionizable cationic lipids.

In some embodiments, the ionizable cationic lipid is a compound of Formula A:

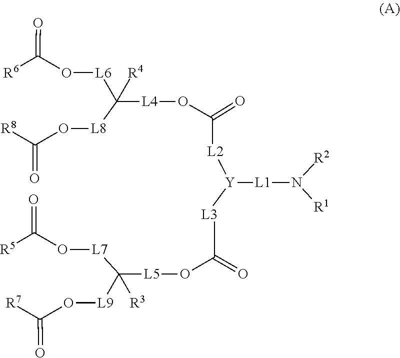

wherein:
R¹ and R² are each independently H or C₁₋₆ alkyl; or
R¹ and R² are joined to form a saturated heterocyclic ring, wherein:
   R¹ is a linear C₁₋₄ alkylene; and
   R² is —(CH₂)ₘ(X)ₙ—, wherein
      X is O, S, or NR⁹, wherein R⁹ is H or C₁₋₆ alkyl;
      m is 1, 2, 3 or 4, and
      n is 0 or 1;
L1 is a linear C₁₋₆ alkylene optionally substituted with one to three methyl groups;
Y is selected from the group consisting of:

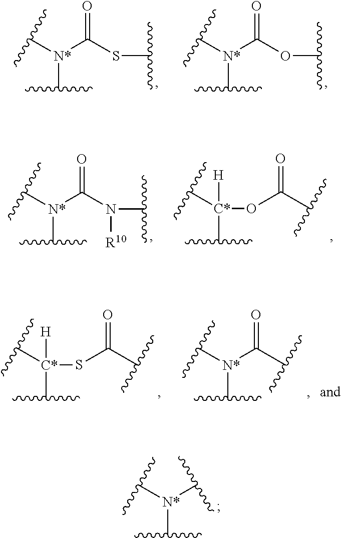

wherein:
   each asterisk (*) indicates the atom attached to L2 and L3; and
   R¹⁰ is H or C₁₋₆ alkyl;
L2 and L3 are each independently a linear C₁₋₈ alkylene;
L4, L5, L6, L7, L8 and L9 are each independently absent or —CH₂—, provided that:
   at least two of L4, L6 and L8 are —CH₂—; and
   at least two of L5, L7 and L9 are —CH₂—;
R³ and R⁴ are each independently H, methyl or ethyl; and
R⁵, R⁶, R⁷ and R⁸ are each independently selected from the group consisting of:
   linear C₁₋₂₀ alkyl, wherein each said linear C₁₋₂₀ alkyl is optionally substituted with one or more substituents selected from the group consisting of:
      C₁₋₆ alkyl, C₁₋₆ alkoxy and —F, wherein each said C₁₋₆ alkyl substituent is optionally substituted with one or more groups selected from the group consisting of C₁₋₃ alkoxy and —F;
      C₃₋₈ monocycloalkyl, wherein each said C₃₋₈ monocycloalkyl is optionally substituted with one or more substituents selected from the group consisting of C₁₋₆ alkyl, C₁₋₆ alkoxy and —F;
      C₇₋₁₂ bicycloalkyl, wherein each said C₇₋₁₂ bicycloalkyl is optionally substituted with one or more substituents selected from the group consisting of C₁₋₆ alkyl, C₁₋₆ alkoxy and —F; and
      C₆₋₁₀ aryl, wherein each said C₆₋₁₀ aryl is a monocyclic or bicyclic aromatic hydrocarbon optionally substituted with one or more substituents selected from the group consisting of C₁₋₆ alkyl, C₁₋₆ alkoxy and —F;
   C₃₋₈ monocycloalkyl, wherein each said C₃₋₈ monocycloalkyl is optionally substituted with one or more substituents selected from the group consisting of C₁₋₆ alkyl, C₁₋₆ alkoxy and —F;
   C₇₋₁₂ bicycloalkyl, wherein each said C₇₋₁₂ bicycloalkyl is optionally substituted with one or more substituents selected from the group consisting of C₁₋₆ alkyl, C₁₋₆ alkoxy and —F; and
   C₆₋₁₀ aryl, wherein each said C₆₋₁₀ aryl is a monocyclic or bicyclic aromatic hydrocarbon optionally substituted with one or more substituents selected from the group consisting of C₁₋₆ alkyl, C₁₋₆ alkoxy and —F.

In some embodiments, the ionizable cationic lipid is a compound of Formula B:

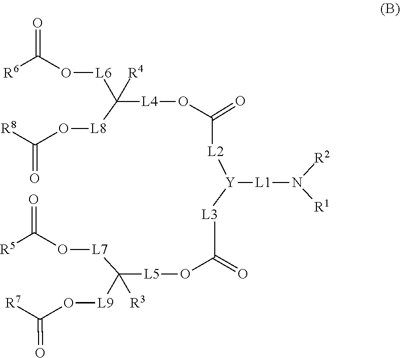

wherein:
R¹ and R² are each independently H or C₁₋₆ alkyl; or
R¹ and R² are joined to form a saturated heterocyclic ring, wherein:
   R¹ is a linear C₁₋₄ alkylene; and
   R² is —(CH₂)ₘ(X)ₙ—, wherein
      X is O, S, or NR⁹, wherein R⁹ is H or C₁₋₆ alkyl;
      m is 1, 2, 3 or 4, and
      n is 0 or 1;
L1 is a linear C₁₋₆ alkylene optionally substituted with one to three methyl groups;
Y is selected from the group consisting of:

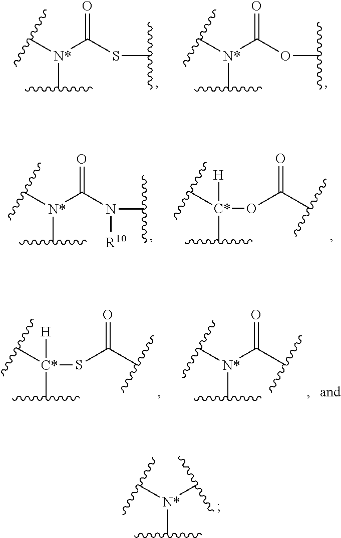

wherein:
   each asterisk (*) indicates the atom attached to L2 and L3; and
   R¹⁰ is H or C₁₋₆ alkyl;
L2 and L3 are each independently a linear C₁₋₈ alkylene;
L4, L5, L6, L7, L8 and L9 are each independently absent or —CH₂—, provided that:
   at least two of L4, L6 and L8 are —CH₂—; and
   at least two of L5, L7 and L9 are —CH₂—;
R³ and R⁴ are each independently H, methyl or ethyl; and
R⁵, R⁶, R⁷ and R⁸ are each independently selected from the group consisting of:
   linear C₁₋₂₀ alkyl, wherein each said linear C₁₋₂₀ alkyl is optionally substituted with one or more substituents selected from the group consisting of:
      C₆₋₁₀ aryl, wherein each said C₆₋₁₀ aryl is a monocyclic or bicyclic aromatic hydrocarbon optionally substituted with one or more C₆₋₁₀ aryl that is optionally substituted with one or more C₁₋₆ alkyl;
      6-10 membered heteroaryl, wherein each said 6-10 membered heteroaryl is a monocyclic or bicyclic aromatic system optionally substituted with one or more C₁₋₆ alkyl;
   C₆₋₁₀ aryl, wherein each said C₆₋₁₀ aryl is a monocyclic or bicyclic aromatic hydrocarbon optionally substituted with one or more C₆₋₁₀ aryl that is optionally substituted with one or more C₁₋₆ alkyl; and
6-10 membered heteroaryl, wherein each said 6-10 membered heteroaryl is a monocyclic or bicyclic aromatic system optionally substituted with one or more C₁₋₆ alkyl.

In some embodiments, the ionizable cationic lipid is a compound of Formula C:

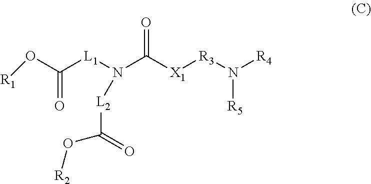

wherein
R₁ is a —CH((CH₂)·CH₃)₂, —CH((CH₂)ₙCH₃)((CH₂)ₙ₋₁CH₃) or —CH((CH₂)_{y}(CH═CH)(CH₂)ₘCH₃)₂, wherein n is 2-8, y is 2-4 and m is 2-4;
R₂ is a linear C₅-C₁₀ alkenyl or a branched C₈-C₁₈ alkyl;
R₃ is a linear C₂-C₆_alkylene;
R₄ and R₅ are the same or different, each a hydrogen or a linear alkyl of 1 to 6 carbons or a branched alkyl of 3 to 6 carbons;
L¹ is a linear C₁-C₈ alkylene;
L₂ is a linear C₁-C₈ alkylene; and
X is S;
   or a pharmaceutically acceptable salt or solvate thereof.

In some embodiments, the ionizable cationic lipid is a compound of Formula D:

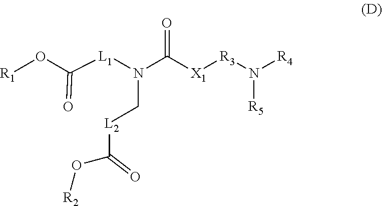

wherein
R₁ is

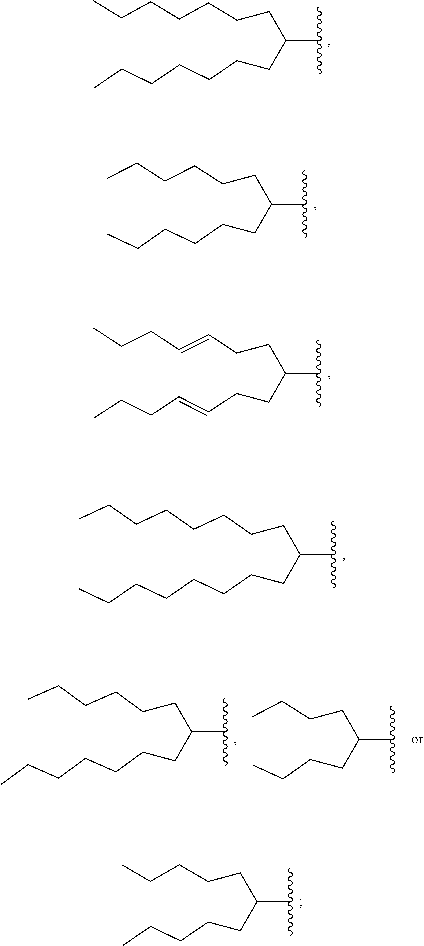

R² is

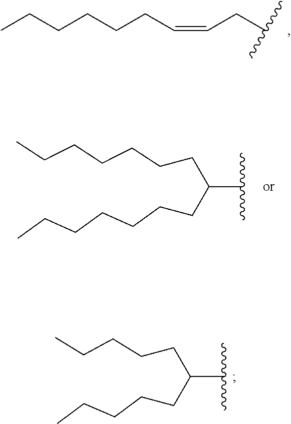

L₁ and L₂ are each independently a linear C₁-C₇ alkyl;
X¹ is O or S;
R³ is a linear C₁-C₆ alkyl; and
R⁴ and R⁵ are each independently a linear C₁-C₆ alkyl;
   or a pharmaceutically acceptable salt thereof.

In some embodiments, the ionizable cationic lipid is a compound selected from Table D, or is a pharmaceutically acceptable salt of a compound selected from Table D.

TABLE D
Exemplary ionizable cationic lipids.

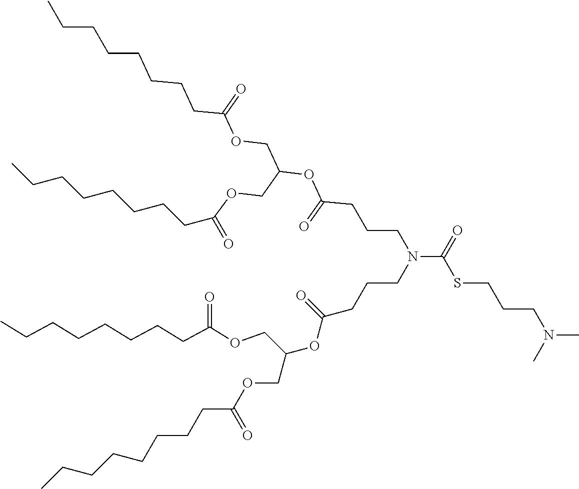 [SMILES: CCCCCCCCC(=O)OCC(COC(=O)CCCCCCCC)OC(=O)CCCN(CCCC(=O)OC(COC(=O)CCCCCCCC)COC(=O)CCCCCCCC)C(=O)SCCCN(C)C]

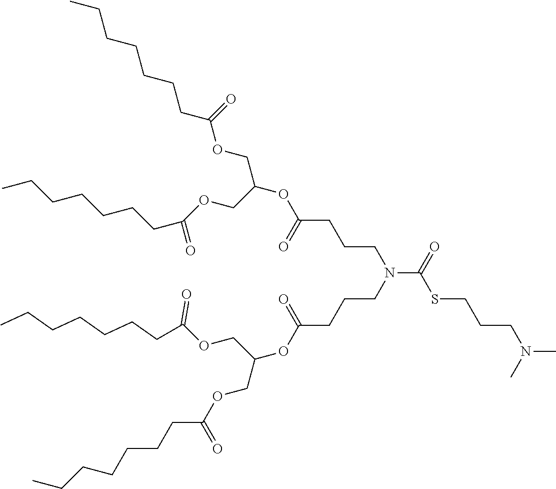 [SMILES: CCCCCCCC(=O)OCC(COC(=O)CCCCCCC)OC(=O)CCCN(CCCC(=O)OC(COC(=O)CCCCCCC)COC(=O)CCCCCCC)C(=O)SCCCN(C)C]

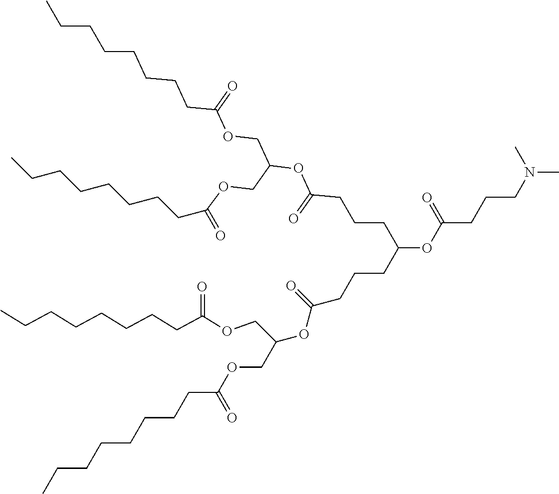 [SMILES: CCCCCCCCC(=O)OCC(COC(=O)CCCCCCCC)OC(=O)CCCC(CCCC(=O)OC(COC(=O)CCCCCCCC)COC(=O)CCCCCCCC)OC(=O)CCCN(C)C]

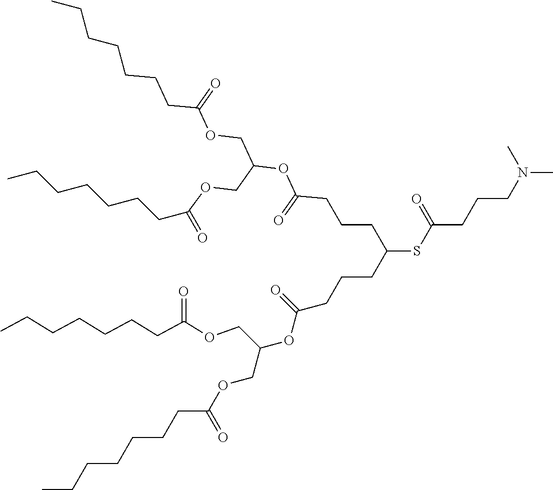 [SMILES: CCCCCCCC(=O)OCC(COC(=O)CCCCCCC)OC(=O)CCCC(CCCC(=O)OC(COC(=O)CCCCCCC)COC(=O)CCCCCCC)SC(=O)CCCN(C)C]

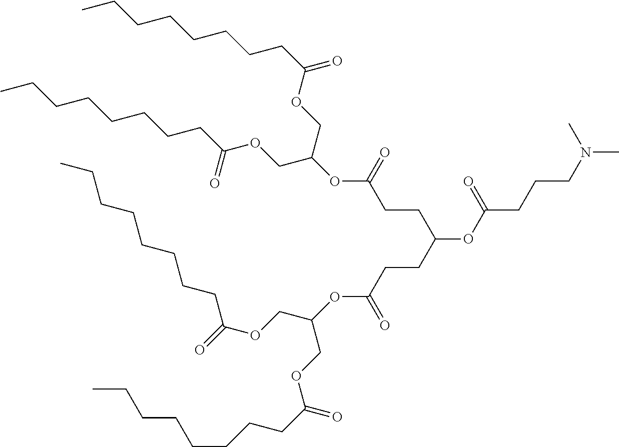 [SMILES: CCCCCCCCC(=O)OCC(COC(=O)CCCCCCCC)OC(=O)CCC(CCC(=O)OC(COC(=O)CCCCCCCC)COC(=O)CCCCCCCC)OC(=O)CCCN(C)C]

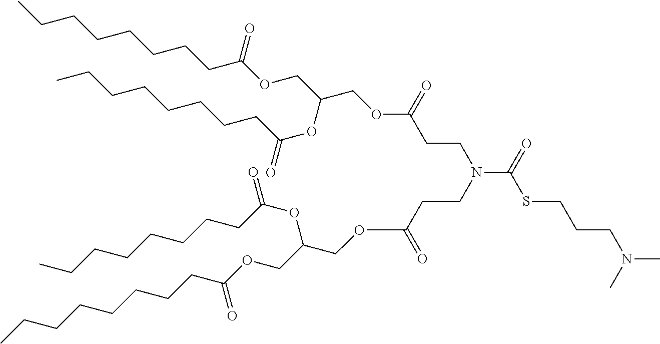 [SMILES: CCCCCCCCC(=O)OCC(COC(=O)CCN(CCC(=O)OCC(COC(=O)CCCCCCCC)OC(=O)CCCCCCCC)C(=O)SCCCN(C)C)OC(=O)CCCCCCCC]

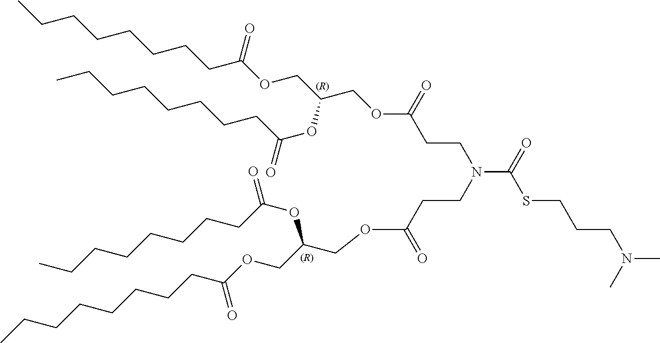

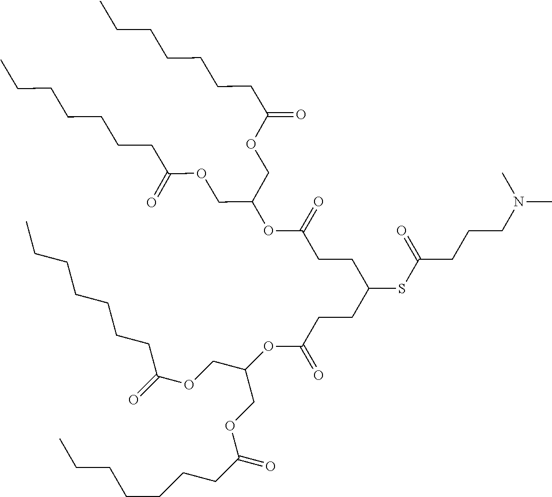 [SMILES: CCCCCCCC(=O)OCC(COC(=O)CCCCCCC)OC(=O)CCC(CCC(=O)OC(COC(=O)CCCCCCC)COC(=O)CCCCCCC)SC(=O)CCCN(C)C]

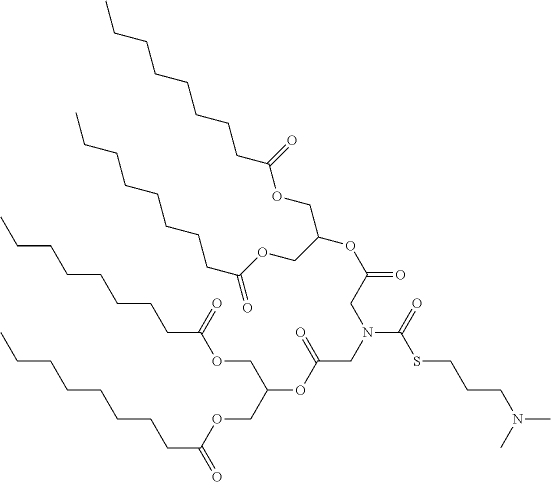 [SMILES: CCCCCCCCC(=O)OCC(COC(=O)CCCCCCCC)OC(=O)CN(CC(=O)OC(COC(=O)CCCCCCCC)COC(=O)CCCCCCCC)C(=O)SCCCN(C)C]

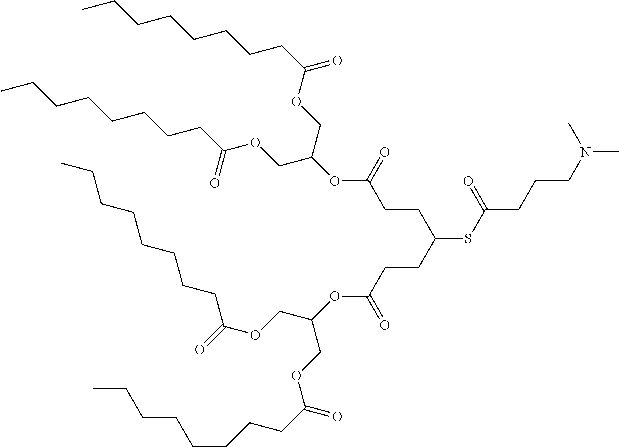 [SMILES: CCCCCCCCC(=O)OCC(COC(=O)CCCCCCCC)OC(=O)CCC(CCC(=O)OC(COC(=O)CCCCCCCC)COC(=O)CCCCCCCC)SC(=O)CCCN(C)C]

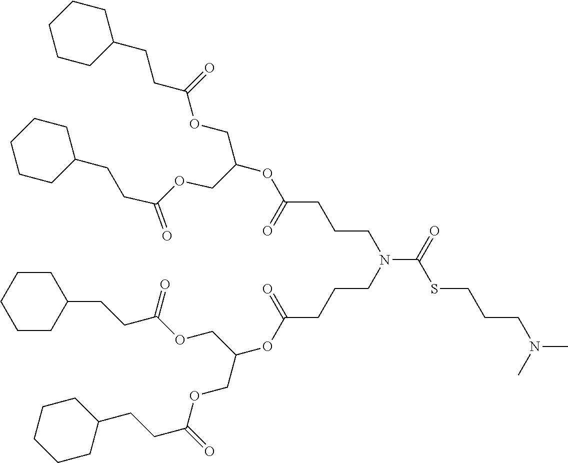 [SMILES: CN(C)CCCSC(=O)N(CCCC(=O)OC(COC(=O)CCC1CCCCC1)COC(=O)CCC1CCCCC1)CCCC(=O)OC(COC(=O)CCC1CCCCC1)COC(=O)CCC1CCCCC1]

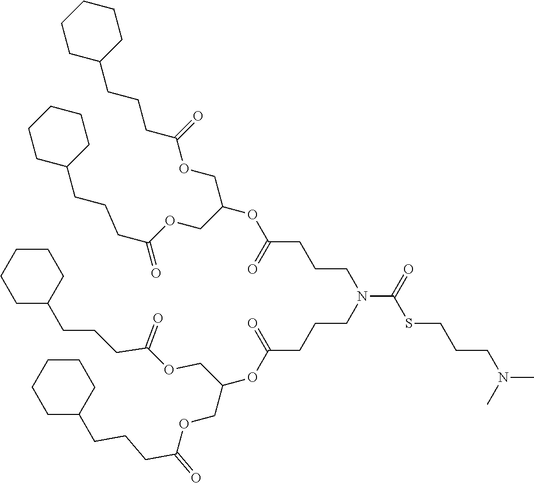 [SMILES: CN(C)CCCSC(=O)N(CCCC(=O)OC(COC(=O)CCCC1CCCCC1)COC(=O)CCCC1CCCCC1)CCCC(=O)OC(COC(=O)CCCC1CCCCC1)COC(=O)CCCC1CCCCC1]

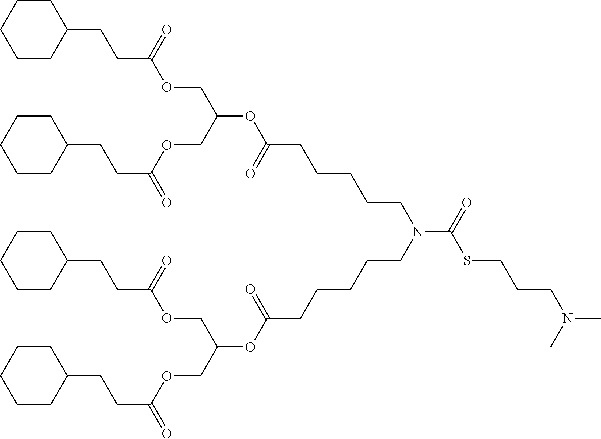 [SMILES: CN(C)CCCSC(=O)N(CCCCCC(=O)OC(COC(=O)CCC1CCCCC1)COC(=O)CCC1CCCCC1)CCCCCC(=O)OC(COC(=O)CCC1CCCCC1)COC(=O)CCC1CCCCC1]

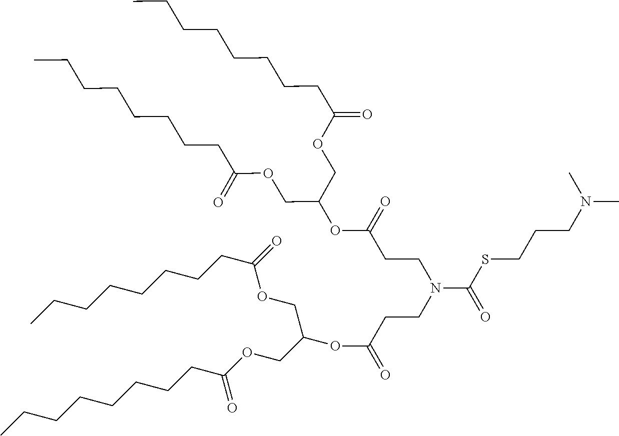 [SMILES: CCCCCCCCC(=O)OCC(COC(=O)CCCCCCCC)OC(=O)CCN(CCC(=O)OC(COC(=O)CCCCCCCC)COC(=O)CCCCCCCC)C(=O)SCCCN(C)C]

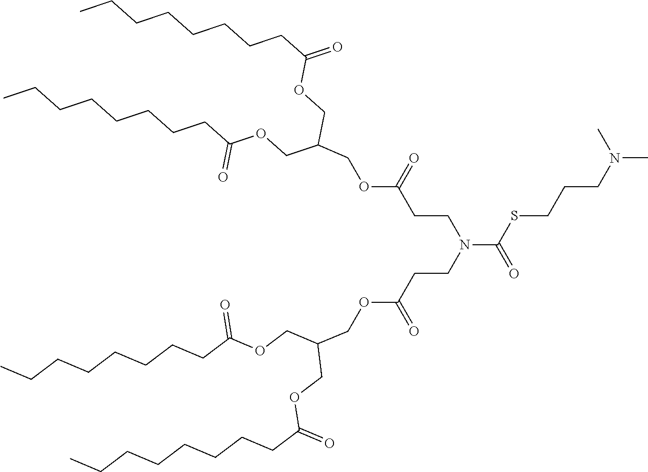 [SMILES: CCCCCCCCC(=O)OCC(COC(=O)CCCCCCCC)COC(=O)CCN(CCC(=O)OCC(COC(=O)CCCCCCCC)COC(=O)CCCCCCCC)C(=O)SCCCN(C)C]

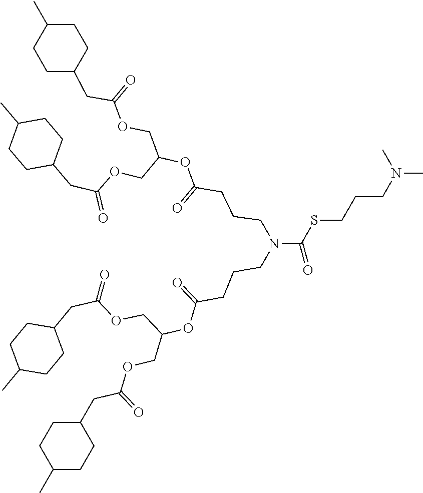

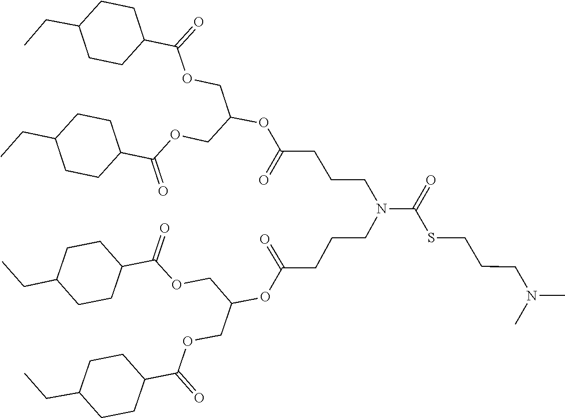 [SMILES: CCC1CCC(C(=O)OCC(COC(=O)C2CCC(CC)CC2)OC(=O)CCCN(CCCC(=O)OC(COC(=O)C2CCC(CC)CC2)COC(=O)C2CCC(CC)CC2)C(=O)SCCCN(C)C)CC1]

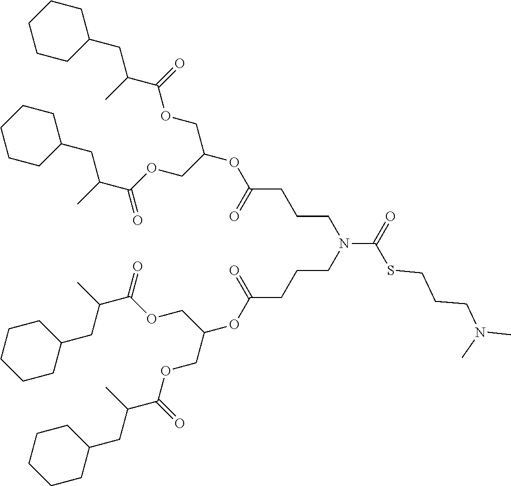 [SMILES: CC(CC1CCCCC1)C(=O)OCC(COC(=O)C(C)CC1CCCCC1)OC(=O)CCCN(CCCC(=O)OC(COC(=O)C(C)CC1CCCCC1)COC(=O)C(C)CC1CCCCC1)C(=O)SCCCN(C)C]

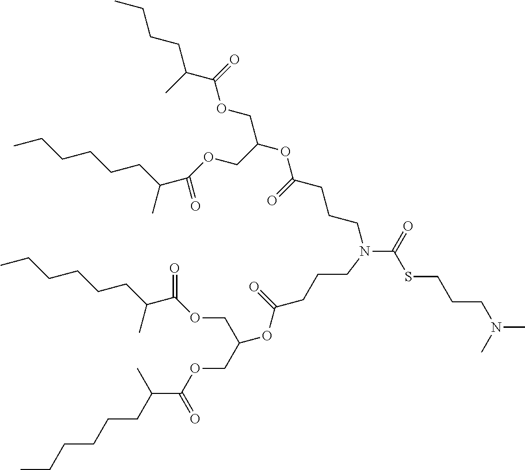 [SMILES: CCCCCCC(C)C(=O)OCC(COC(=O)C(C)CCCC)OC(=O)CCCN(CCCC(=O)OC(COC(=O)C(C)CCCCCC)COC(=O)C(C)CCCCCC)C(=O)SCCCN(C)C]

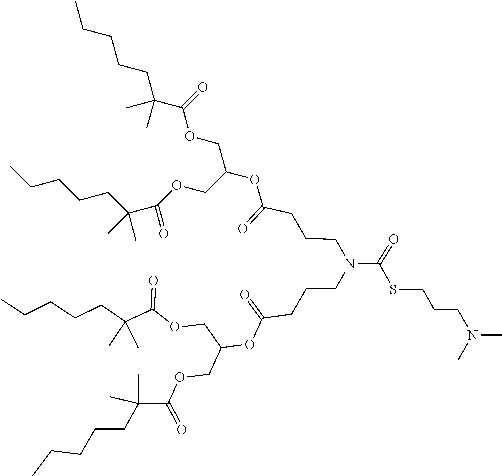 [SMILES: CCCCCC(C)(C)C(=O)OCC(COC(=O)C(C)(C)CCCCC)OC(=O)CCCN(CCCC(=O)OC(COC(=O)C(C)(C)CCCCC)COC(=O)C(C)(C)CCCCC)C(=O)SCCCN(C)C]

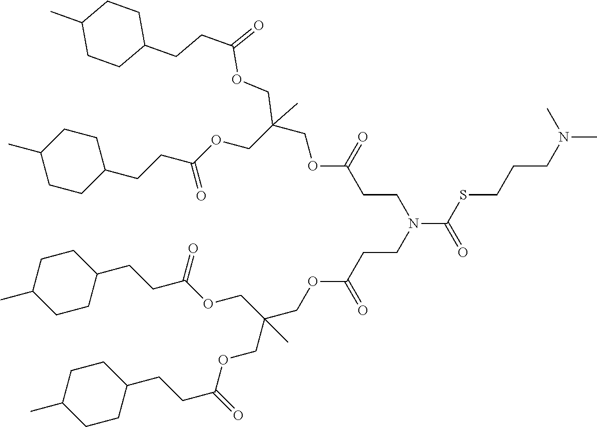

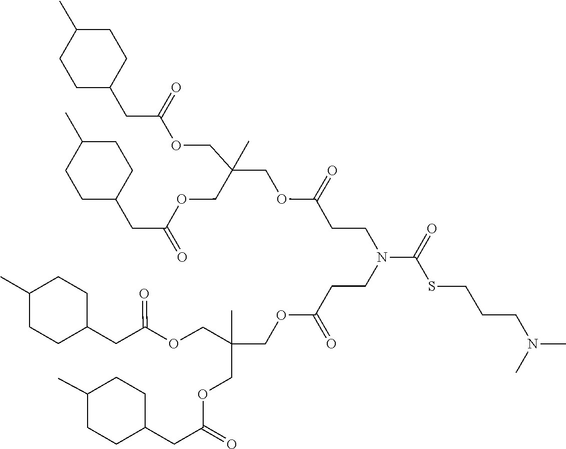

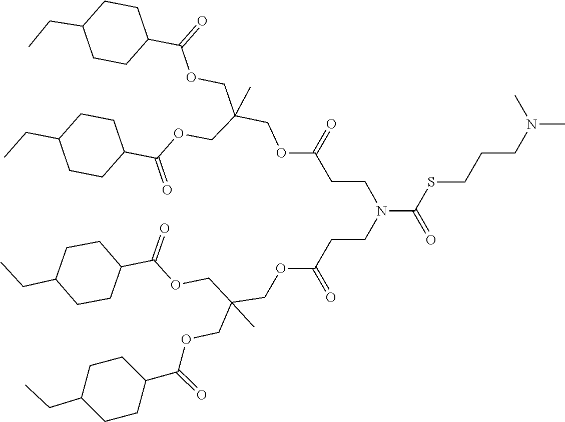 [SMILES: CCC1CCC(C(=O)OCC(C)(COC(=O)CCN(CCC(=O)OCC(C)(COC(=O)C2CCC(CC)CC2)COC(=O)C2CCC(CC)CC2)C(=O)SCCCN(C)C)COC(=O)C2CCC(CC)CC2)CC1]

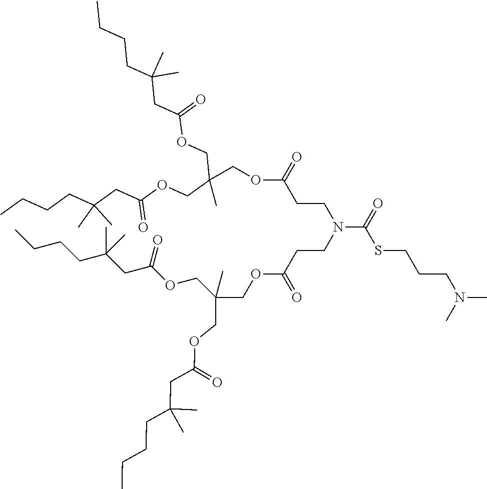 [SMILES: CCCCC(C)(C)CC(=O)OCC(C)(COC(=O)CCN(CCC(=O)OCC(C)(COC(=O)CC(C)(C)CCCC)COC(=O)CC(C)(C)CCCC)C(=O)SCCCN(C)C)COC(=O)CC(C)(C)CCCC]

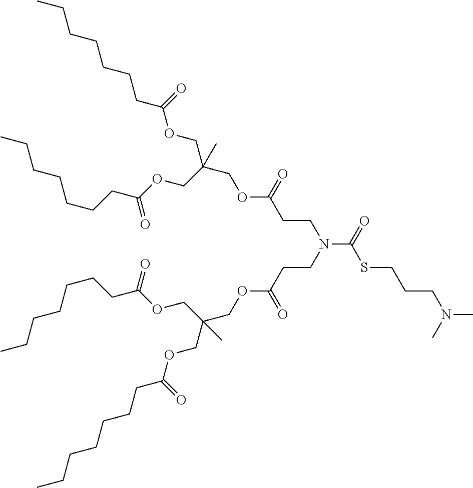 [SMILES: CCCCCCCC(=O)OCC(C)(COC(=O)CCCCCCC)COC(=O)CCN(CCC(=O)OCC(C)(COC(=O)CCCCCCC)COC(=O)CCCCCCC)C(=O)SCCCN(C)C]

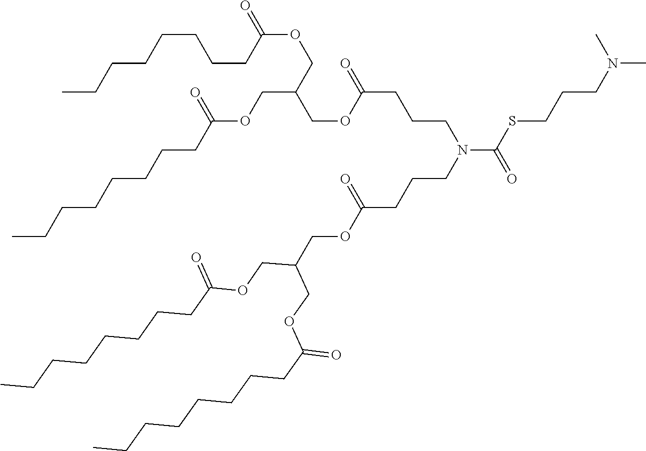 [SMILES: CCCCCCCCC(=O)OCC(COC(=O)CCCCCCCC)COC(=O)CCCN(CCCC(=O)OCC(COC(=O)CCCCCCCC)COC(=O)CCCCCCCC)C(=O)SCCCN(C)C]

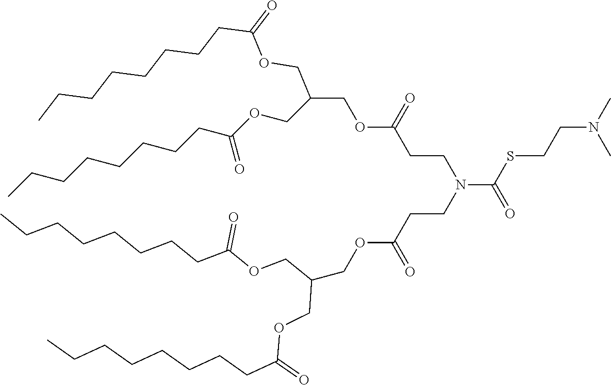 [SMILES: CCCCCCCCC(=O)OCC(COC(=O)CCCCCCCC)COC(=O)CCN(CCC(=O)OCC(COC(=O)CCCCCCCC)COC(=O)CCCCCCCC)C(=O)SCCN(C)C]

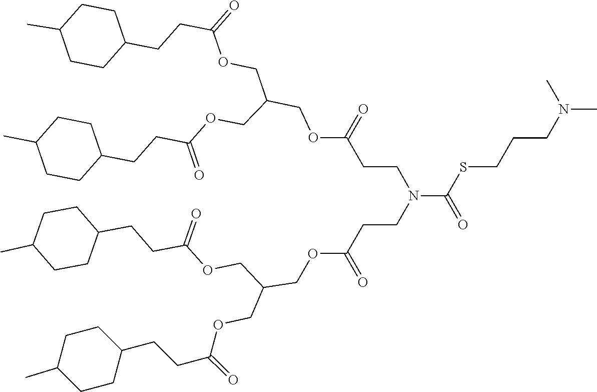

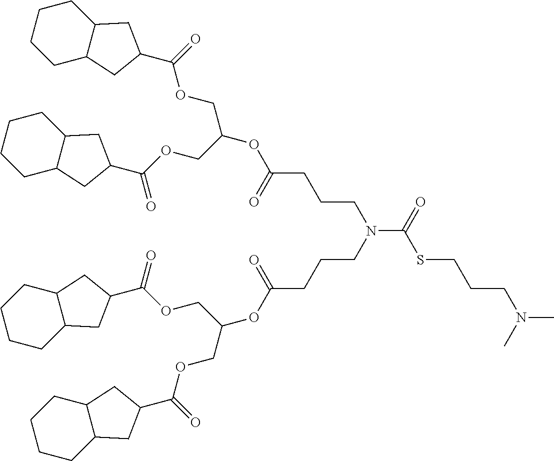 [SMILES: CN(C)CCCSC(=O)N(CCCC(=O)OC(COC(=O)C1CC2CCCCC2C1)COC(=O)C1CC2CCCCC2C1)CCCC(=O)OC(COC(=O)C1CC2CCCCC2C1)COC(=O)C1CC2CCCCC2C1]

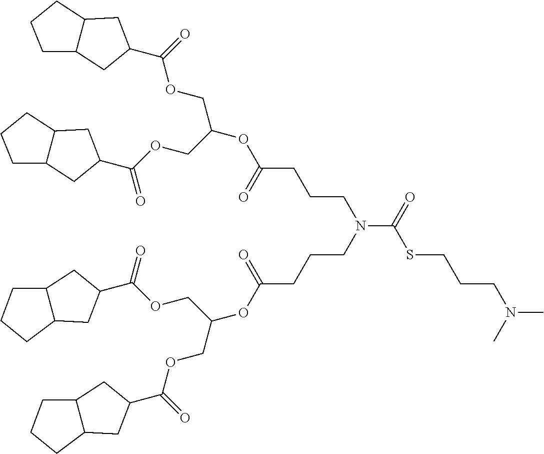 [SMILES: CN(C)CCCSC(=O)N(CCCC(=O)OC(COC(=O)C1CC2CCCC2C1)COC(=O)C1CC2CCCC2C1)CCCC(=O)OC(COC(=O)C1CC2CCCC2C1)COC(=O)C1CC2CCCC2C1]

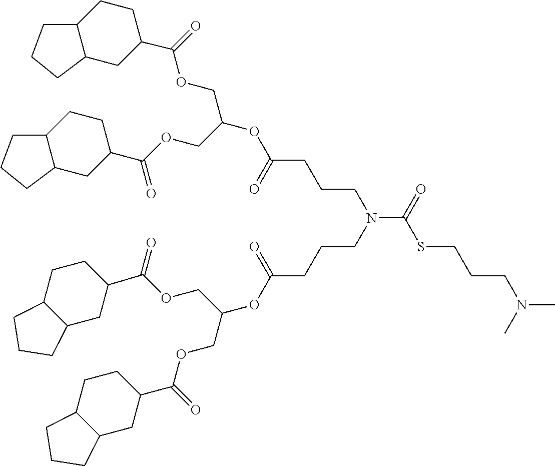 [SMILES: CN(C)CCCSC(=O)N(CCCC(=O)OC(COC(=O)C1CCC2CCCC2C1)COC(=O)C1CCC2CCCC2C1)CCCC(=O)OC(COC(=O)C1CCC2CCCC2C1)COC(=O)C1CCC2CCCC2C1]

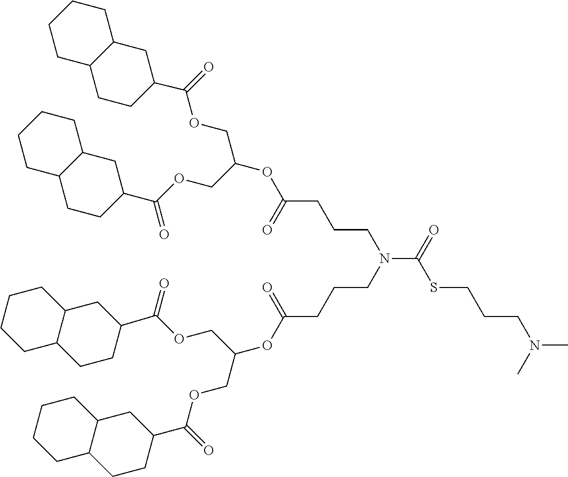 [SMILES: CN(C)CCCSC(=O)N(CCCC(=O)OC(COC(=O)C1CCC2CCCCC2C1)COC(=O)C1CCC2CCCCC2C1)CCCC(=O)OC(COC(=O)C1CCC2CCCCC2C1)COC(=O)C1CCC2CCCCC2C1]

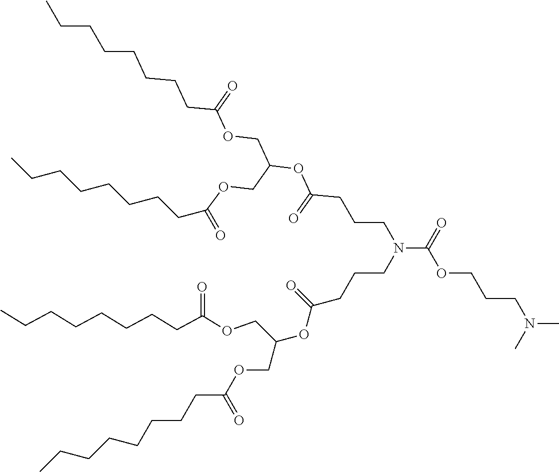 [SMILES: CCCCCCCCC(=O)OCC(COC(=O)CCCCCCCC)OC(=O)CCCN(CCCC(=O)OC(COC(=O)CCCCCCCC)COC(=O)CCCCCCCC)C(=O)OCCCN(C)C]

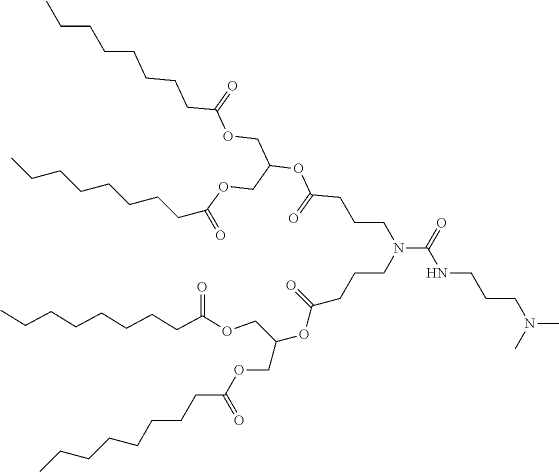 [SMILES: CCCCCCCCC(=O)OCC(COC(=O)CCCCCCCC)OC(=O)CCCN(CCCC(=O)OC(COC(=O)CCCCCCCC)COC(=O)CCCCCCCC)C(=O)NCCCN(C)C]

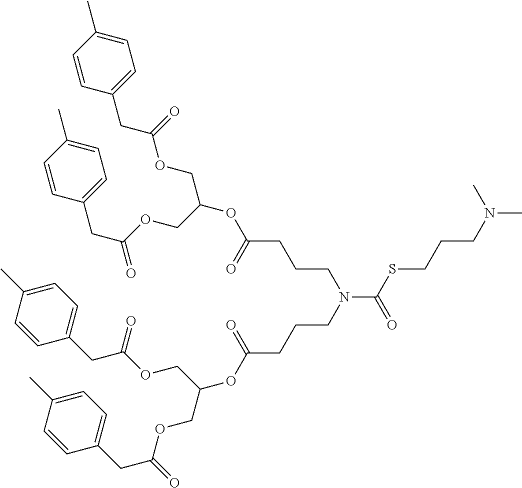

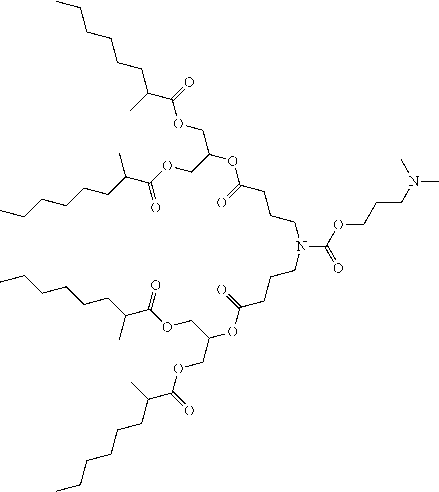 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)OCCCN(C)C]

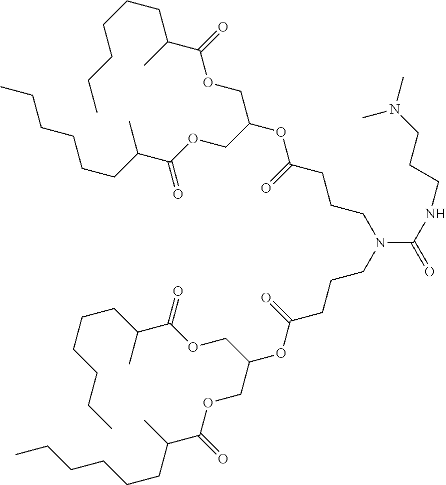 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)NCCCN(C)C]

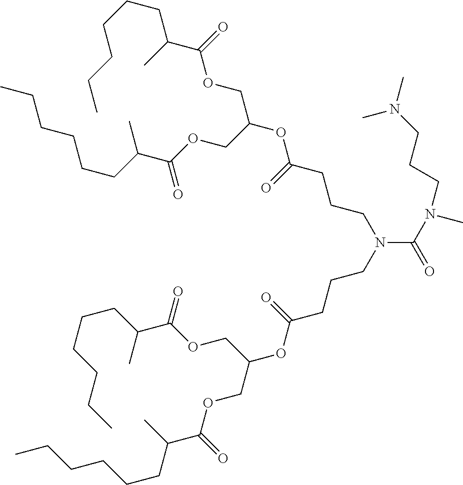 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)N(C)CCCN(C)C]

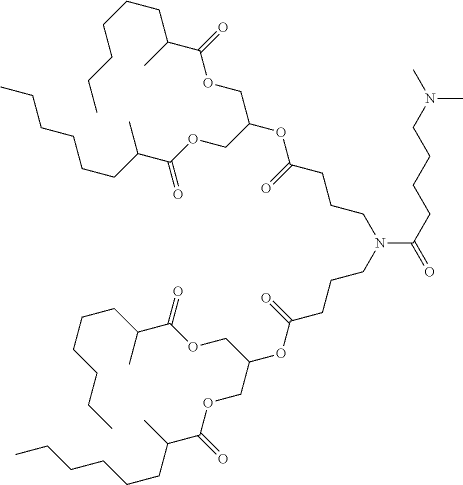 [SMILES: CCCCCCC(C)C(=O)OCC(COC(=O)C(C)CCCCCC)OC(=O)CCCN(CCCC(=O)OC(COC(=O)C(C)CCCCCC)COC(=O)C(C)CCCCCC)C(=O)CCCCN(C)C]

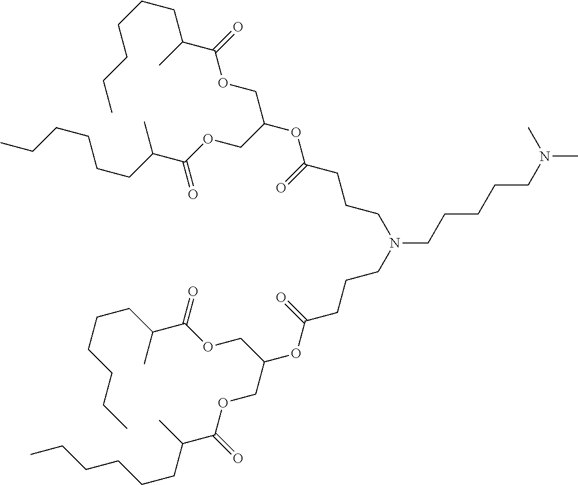 [SMILES: CCCCCCC(C)C(=O)OCC(COC(=O)C(C)CCCCCC)OC(=O)CCCN(CCCCCN(C)C)CCCC(=O)OC(COC(=O)C(C)CCCCCC)COC(=O)C(C)CCCCCC]

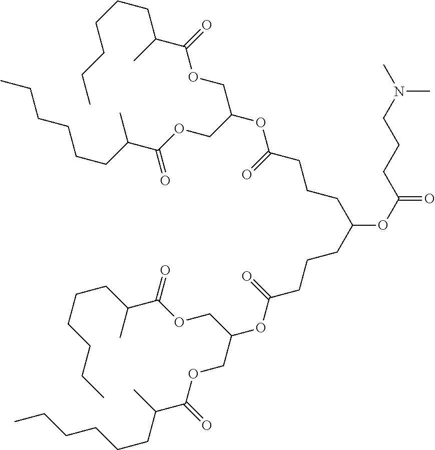 [SMILES: CCCCCCC(C)C(=O)OCC(COC(=O)C(C)CCCCCC)OC(=O)CCCC(CCCC(=O)OC(COC(=O)C(C)CCCCCC)COC(=O)C(C)CCCCCC)OC(=O)CCCN(C)C]

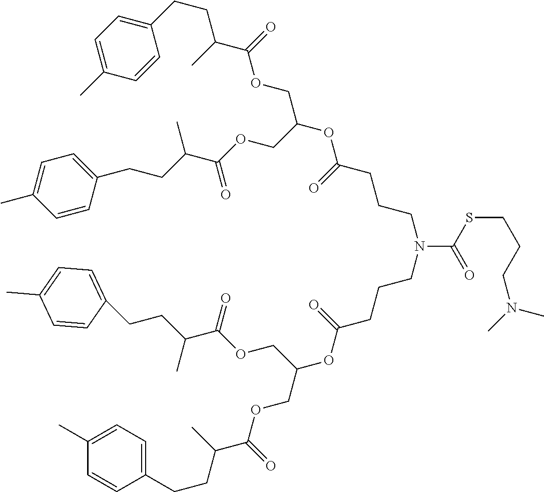

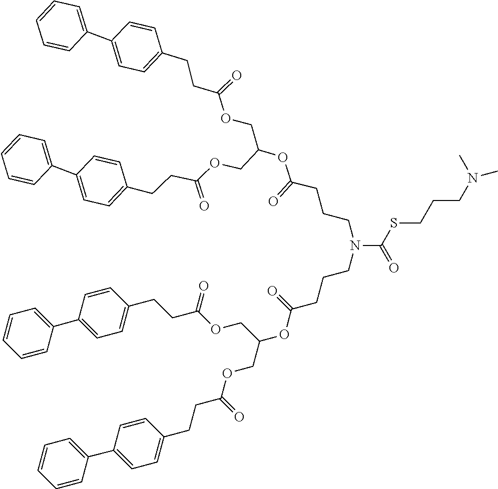 [SMILES: CN(C)CCCSC(=O)N(CCCC(=O)OC(COC(=O)CCc1ccc(-c2ccccc2)cc1)COC(=O)CCc1ccc(-c2ccccc2)cc1)CCCC(=O)OC(COC(=O)CCc1ccc(-c2ccccc2)cc1)COC(=O)CCc1ccc(-c2ccccc2)cc1]

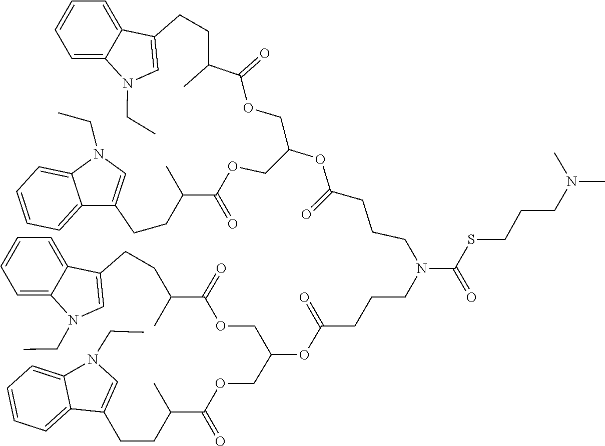 [SMILES: CCn1cc(CCC(C)C(=O)OCC(COC(=O)C(C)CCc2cn(CC)c3ccccc23)OC(=O)CCCN(CCCC(=O)OC(COC(=O)C(C)CCc2cn(CC)c3ccccc23)COC(=O)C(C)CCc2cn(CC)c3ccccc23)C(=O)SCCCN(C)C)c2ccccc21]

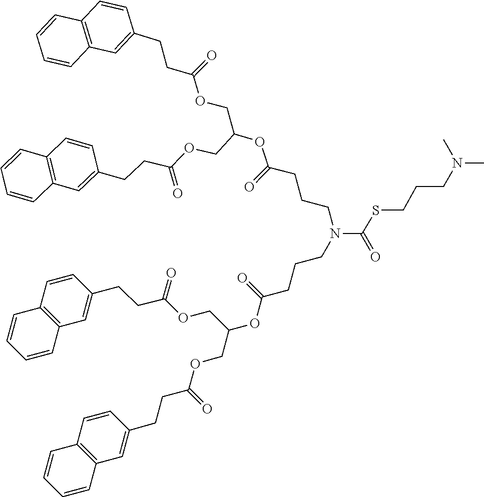 [SMILES: CN(C)CCCSC(=O)N(CCCC(=O)OC(COC(=O)CCc1ccc2ccccc2c1)COC(=O)CCc1ccc2ccccc2c1)CCCC(=O)OC(COC(=O)CCc1ccc2ccccc2c1)COC(=O)CCc1ccc2ccccc2c1]

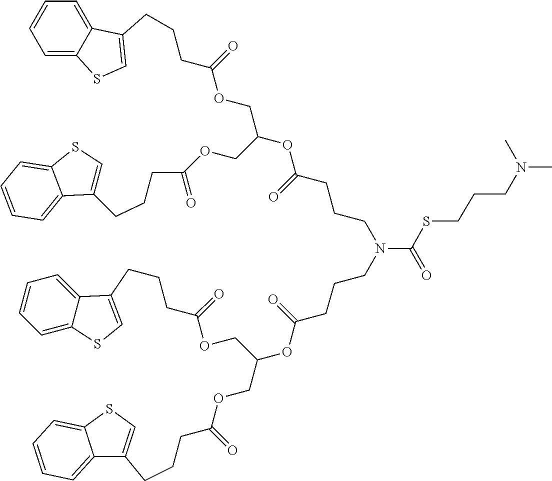 [SMILES: CN(C)CCCSC(=O)N(CCCC(=O)OC(COC(=O)CCCc1csc2ccccc12)COC(=O)CCCc1csc2ccccc12)CCCC(=O)OC(COC(=O)CCCc1csc2ccccc12)COC(=O)CCCc1csc2ccccc12]

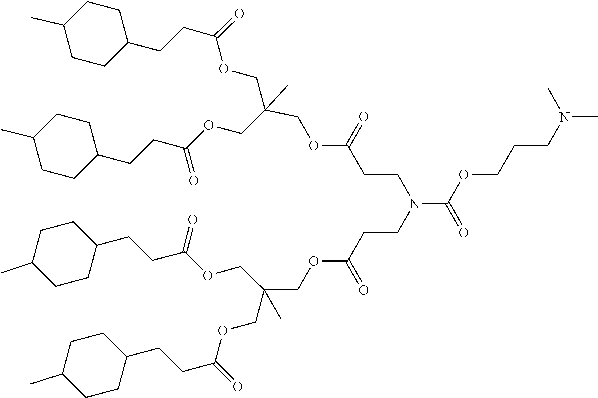

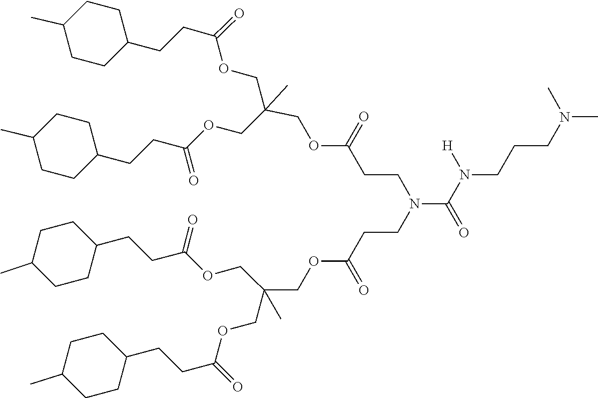

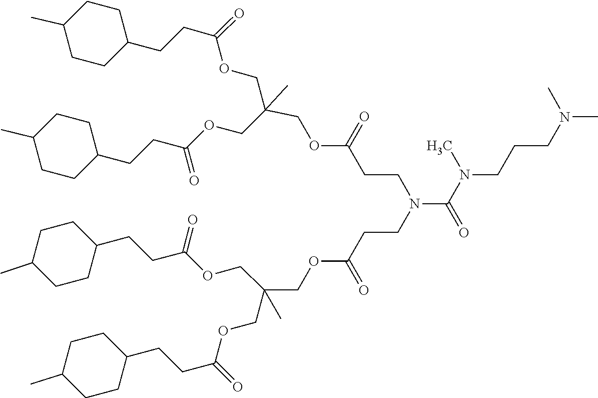

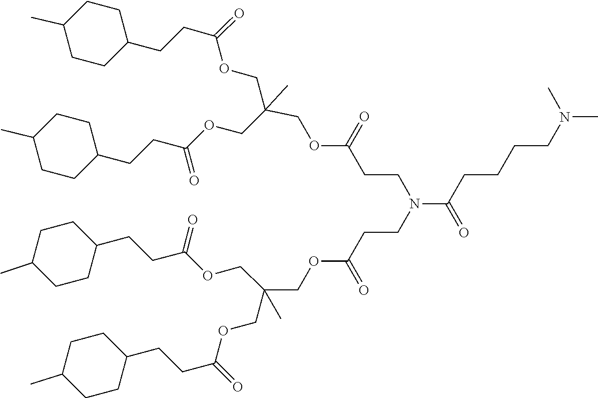

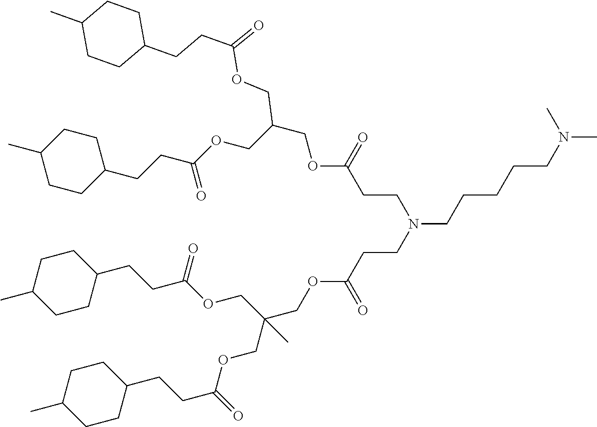

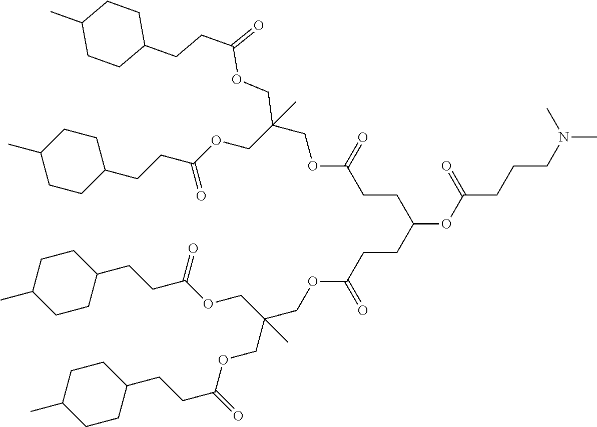

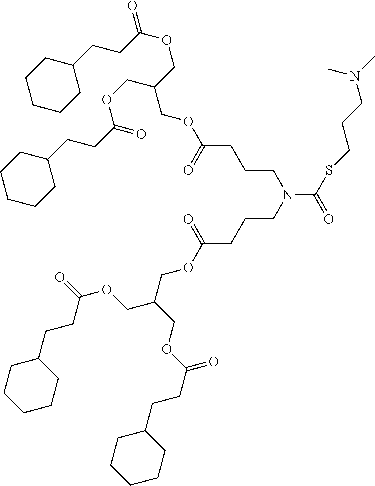 [SMILES: CN(C)CCCSC(=O)N(CCCC(=O)OCC(COC(=O)CCC1CCCCC1)COC(=O)CCC1CCCCC1)CCCC(=O)OCC(COC(=O)CCC1CCCCC1)COC(=O)CCC1CCCCC1]

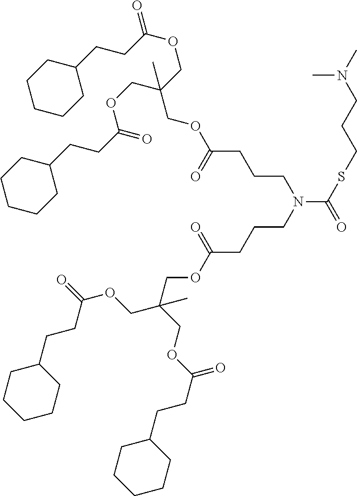 [SMILES: CN(C)CCCSC(=O)N(CCCC(=O)OCC(C)(COC(=O)CCC1CCCCC1)COC(=O)CCC1CCCCC1)CCCC(=O)OCC(C)(COC(=O)CCC1CCCCC1)COC(=O)CCC1CCCCC1]

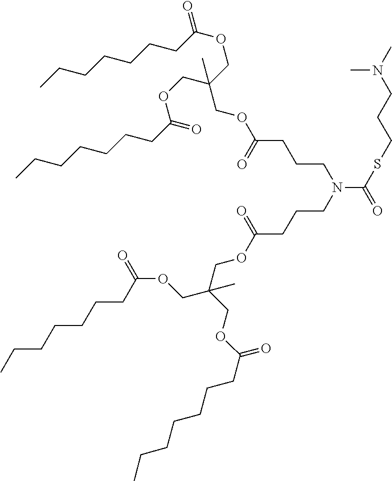 [SMILES: CCCCCCCC(=O)OCC(C)(COC(=O)CCCCCCC)COC(=O)CCCN(CCCC(=O)OCC(C)(COC(=O)CCCCCCC)COC(=O)CCCCCCC)C(=O)SCCCN(C)C]

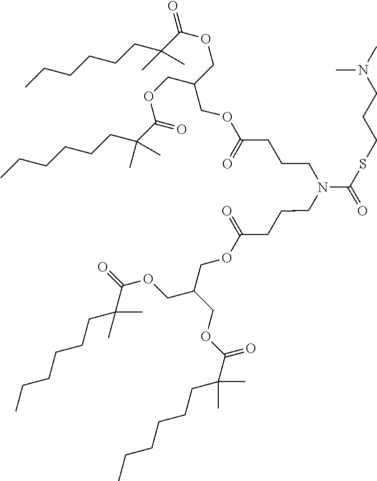 [SMILES: CCCCCCC(C)(C)C(=O)OCC(COC(=O)CCCN(CCCC(=O)OCC(COC(=O)C(C)(C)CCCCCC)COC(=O)C(C)(C)CCCCCC)C(=O)SCCCN(C)C)COC(=O)C(C)(C)CCCCCC]

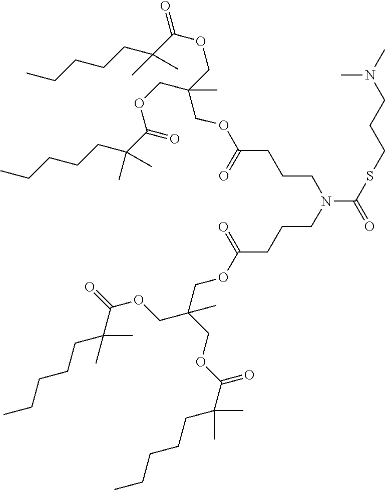 [SMILES: CCCCCC(C)(C)C(=O)OCC(C)(COC(=O)CCCN(CCCC(=O)OCC(C)(COC(=O)C(C)(C)CCCCC)COC(=O)C(C)(C)CCCCC)C(=O)SCCCN(C)C)COC(=O)C(C)(C)CCCCC]

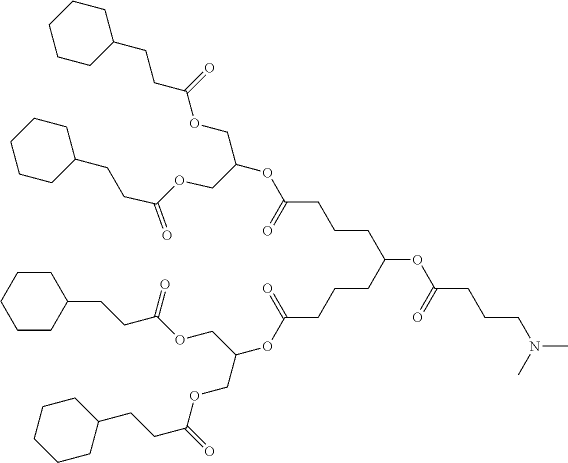 [SMILES: CN(C)CCCC(=O)OC(CCCC(=O)OC(COC(=O)CCC1CCCCC1)COC(=O)CCC1CCCCC1)CCCC(=O)OC(COC(=O)CCC1CCCCC1)COC(=O)CCC1CCCCC1]

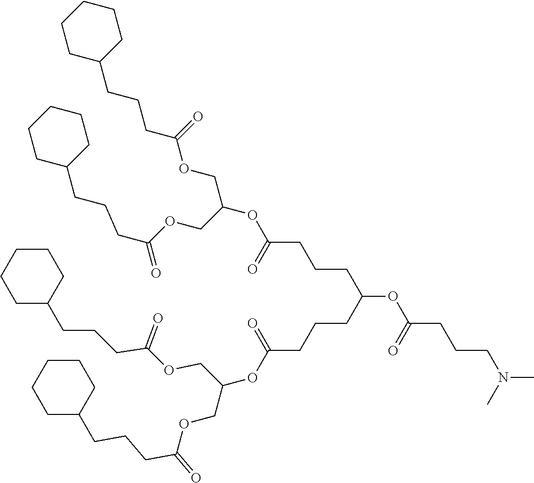 [SMILES: CN(C)CCCC(=O)OC(CCCC(=O)OC(COC(=O)CCCC1CCCCC1)COC(=O)CCCC1CCCCC1)CCCC(=O)OC(COC(=O)CCCC1CCCCC1)COC(=O)CCCC1CCCCC1]

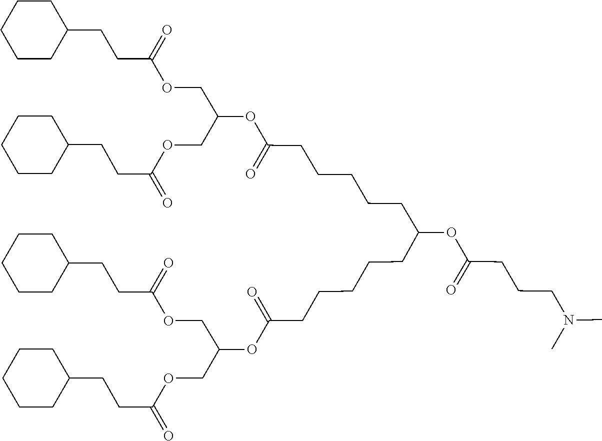 [SMILES: CN(C)CCCC(=O)OC(CCCCCC(=O)OC(COC(=O)CCC1CCCCC1)COC(=O)CCC1CCCCC1)CCCCCC(=O)OC(COC(=O)CCC1CCCCC1)COC(=O)CCC1CCCCC1]

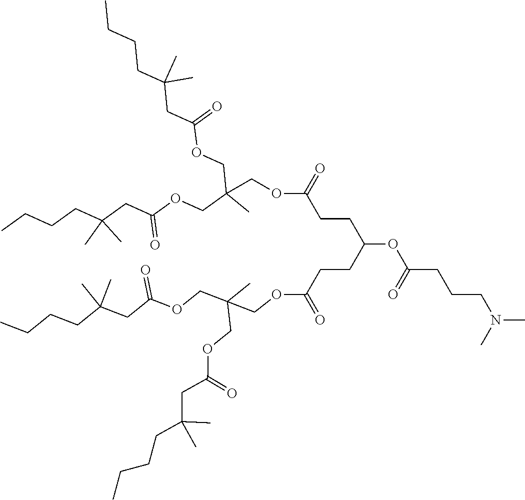 [SMILES: CCCCC(C)(C)CC(=O)OCC(C)(COC(=O)CCC(CCC(=O)OCC(C)(COC(=O)CC(C)(C)CCCC)COC(=O)CC(C)(C)CCCC)OC(=O)CCCN(C)C)COC(=O)CC(C)(C)CCCC]

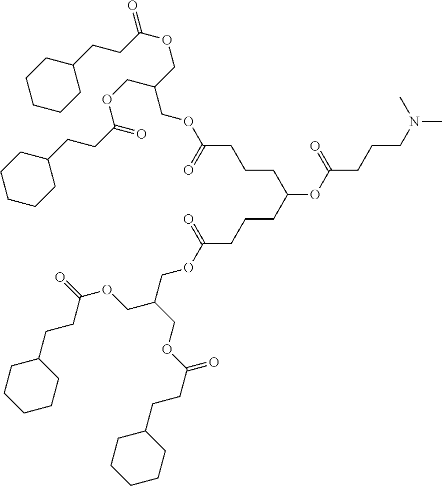 [SMILES: CN(C)CCCC(=O)OC(CCCC(=O)OCC(COC(=O)CCC1CCCCC1)COC(=O)CCC1CCCCC1)CCCC(=O)OCC(COC(=O)CCC1CCCCC1)COC(=O)CCC1CCCCC1]

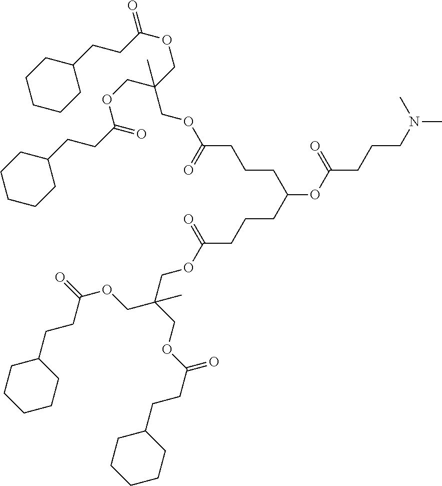 [SMILES: CN(C)CCCC(=O)OC(CCCC(=O)OCC(C)(COC(=O)CCC1CCCCC1)COC(=O)CCC1CCCCC1)CCCC(=O)OCC(C)(COC(=O)CCC1CCCCC1)COC(=O)CCC1CCCCC1]

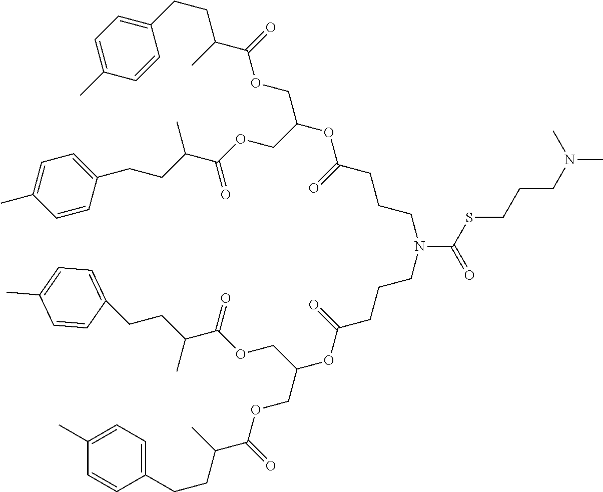

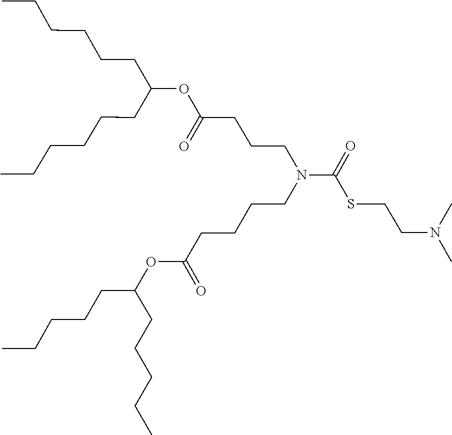 [SMILES: CCCCCCC(CCCCCC)OC(=O)CCCN(CCCCC(=O)OC(CCCCC)CCCCC)C(=O)SCCN(C)C]

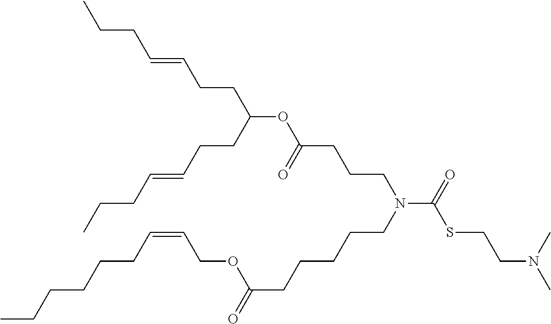 [SMILES: CCC/C=C/CCC(CC/C=C/CCC)OC(=O)CCCN(CCCCCC(=O)OC/C=C\CCCCCC)C(=O)SCCN(C)C]

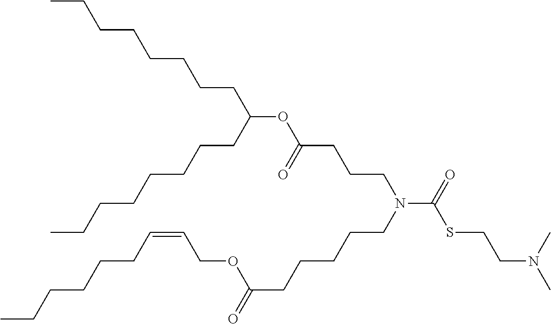 [SMILES: CCCCCC/C=C\COC(=O)CCCCCN(CCCC(=O)OC(CCCCCCCC)CCCCCCCC)C(=O)SCCN(C)C]

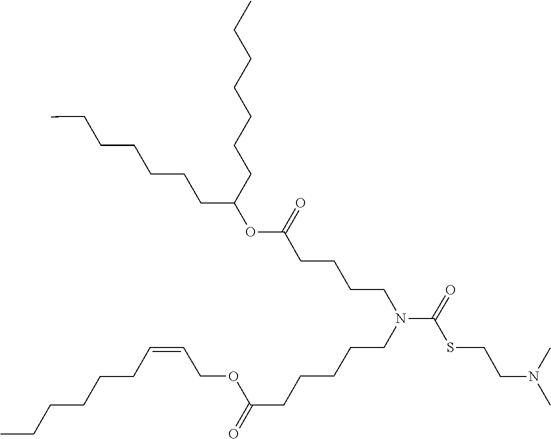 [SMILES: CCCCCC/C=C\COC(=O)CCCCCN(CCCCC(=O)OC(CCCCCCC)CCCCCCC)C(=O)SCCN(C)C]

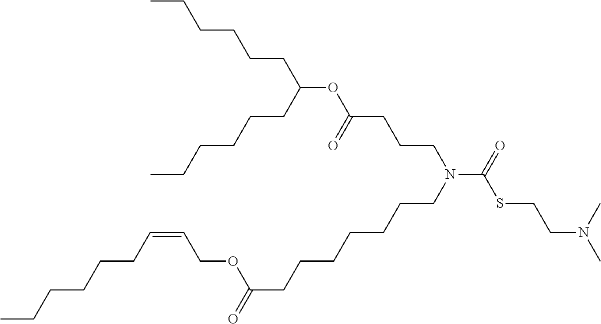 [SMILES: CCCCCC/C=C\COC(=O)CCCCCCCN(CCCC(=O)OC(CCCCCC)CCCCCC)C(=O)SCCN(C)C]

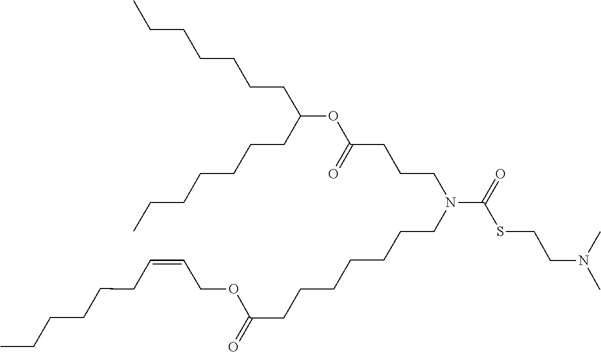 [SMILES: CCCCCC/C=C\COC(=O)CCCCCCCN(CCCC(=O)OC(CCCCCCC)CCCCCCC)C(=O)SCCN(C)C]

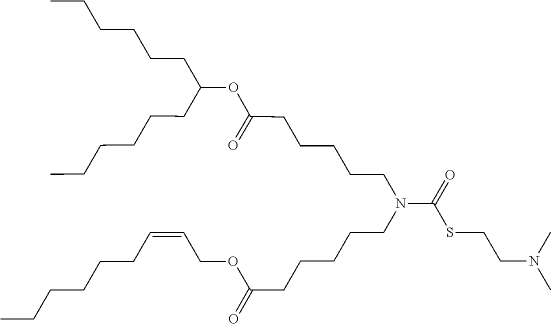 [SMILES: CCCCCC/C=C\COC(=O)CCCCCN(CCCCCC(=O)OC(CCCCCC)CCCCCC)C(=O)SCCN(C)C]

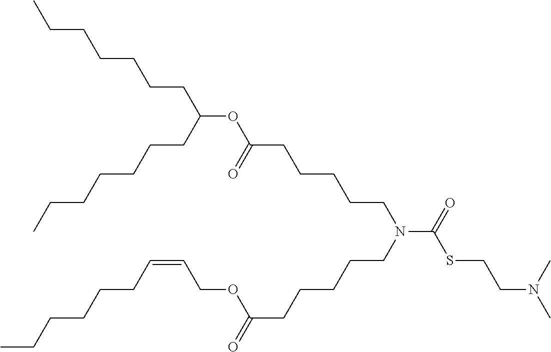 [SMILES: CCCCCC/C=C\COC(=O)CCCCCN(CCCCCC(=O)OC(CCCCCCC)CCCCCCC)C(=O)SCCN(C)C]

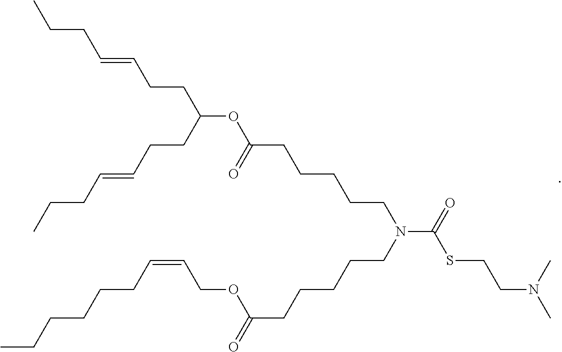 [SMILES: CCC/C=C/CCC(CC/C=C/CCC)OC(=O)CCCCCN(CCCCCC(=O)OC/C=C\CCCCCC)C(=O)SCCN(C)C]

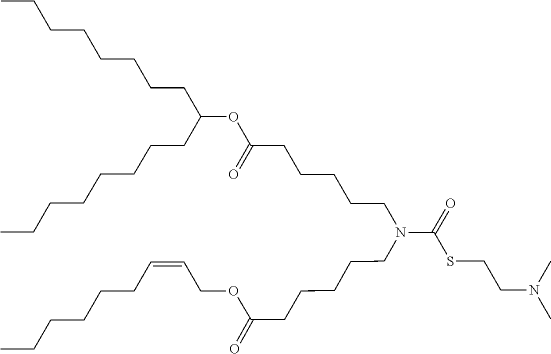 [SMILES: CCCCCC/C=C\COC(=O)CCCCCN(CCCCCC(=O)OC(CCCCCCCC)CCCCCCCC)C(=O)SCCN(C)C]

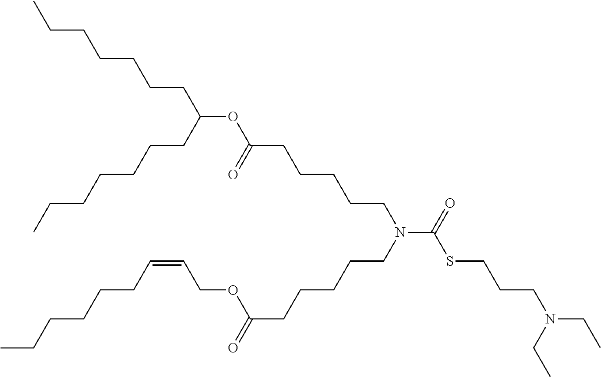 [SMILES: CCCCCC/C=C\COC(=O)CCCCCN(CCCCCC(=O)OC(CCCCCCC)CCCCCCC)C(=O)SCCCN(CC)CC]

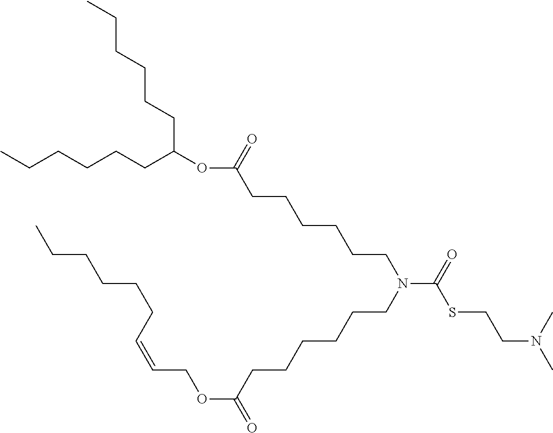 [SMILES: CCCCCC/C=C\COC(=O)CCCCCCN(CCCCCCC(=O)OC(CCCCCC)CCCCCC)C(=O)SCCN(C)C]

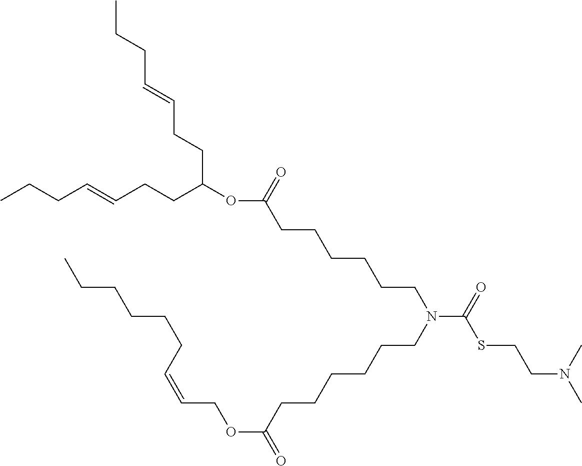 [SMILES: CCC/C=C/CCC(CC/C=C/CCC)OC(=O)CCCCCCN(CCCCCCC(=O)OC/C=C\CCCCCC)C(=O)SCCN(C)C]

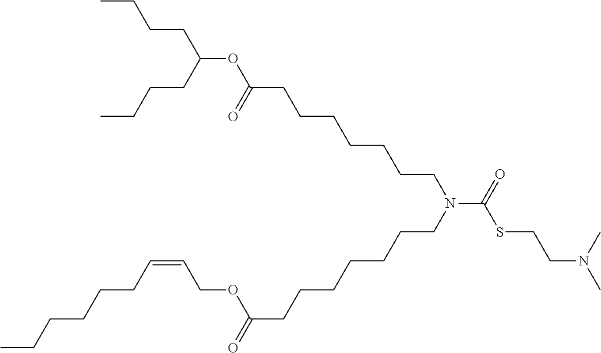 [SMILES: CCCCCC/C=C\COC(=O)CCCCCCCN(CCCCCCCC(=O)OC(CCCC)CCCC)C(=O)SCCN(C)C]

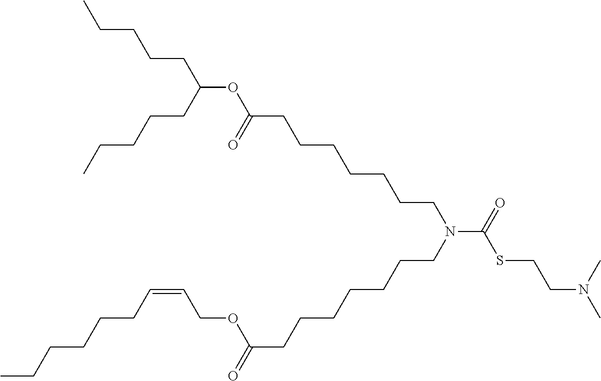 [SMILES: CCCCCC/C=C\COC(=O)CCCCCCCN(CCCCCCCC(=O)OC(CCCCC)CCCCC)C(=O)SCCN(C)C]

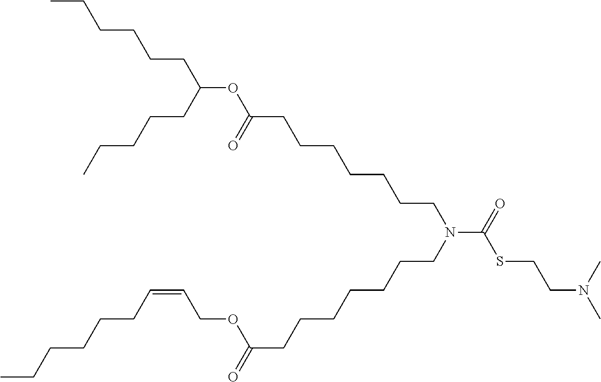 [SMILES: CCCCCC/C=C\COC(=O)CCCCCCCN(CCCCCCCC(=O)OC(CCCCC)CCCCCC)C(=O)SCCN(C)C]

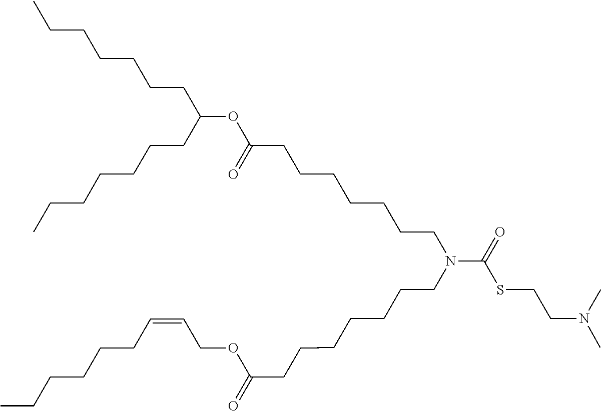 [SMILES: CCCCCC/C=C\COC(=O)CCCCCCCN(CCCCCCCC(=O)OC(CCCCCCC)CCCCCCC)C(=O)SCCN(C)C]

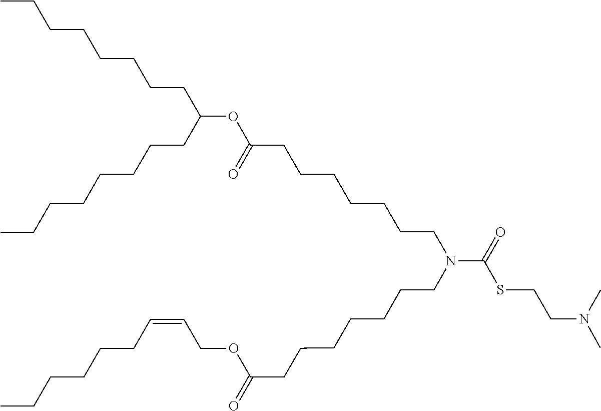 [SMILES: CCCCCC/C=C\COC(=O)CCCCCCCN(CCCCCCCC(=O)OC(CCCCCCCC)CCCCCCCC)C(=O)SCCN(C)C]

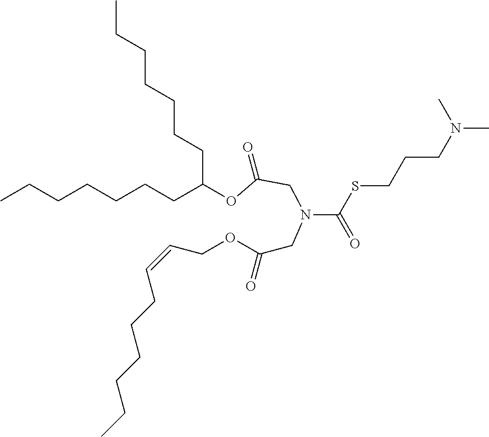 [SMILES: CCCCCC/C=C\COC(=O)CN(CC(=O)OC(CCCCCCC)CCCCCCC)C(=O)SCCCN(C)C]

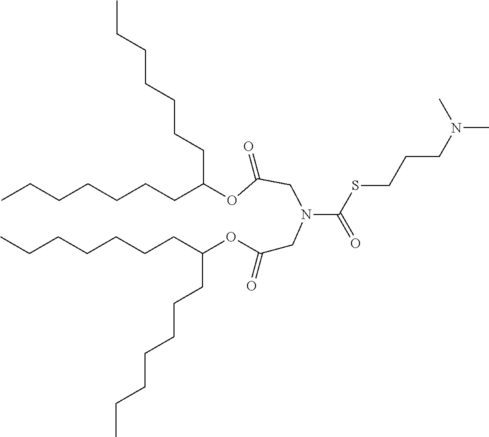 [SMILES: CCCCCCCC(CCCCCCC)OC(=O)CN(CC(=O)OC(CCCCCCC)CCCCCCC)C(=O)SCCCN(C)C]

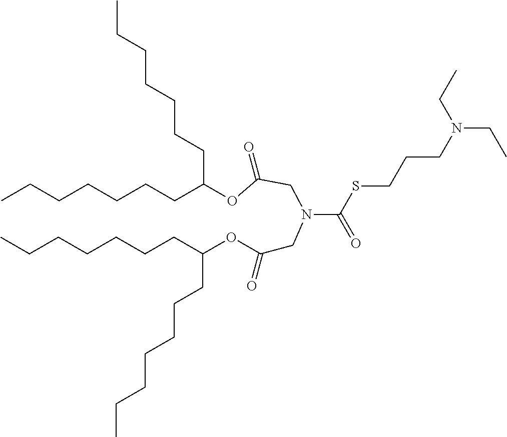 [SMILES: CCCCCCCC(CCCCCCC)OC(=O)CN(CC(=O)OC(CCCCCCC)CCCCCCC)C(=O)SCCCN(CC)CC]

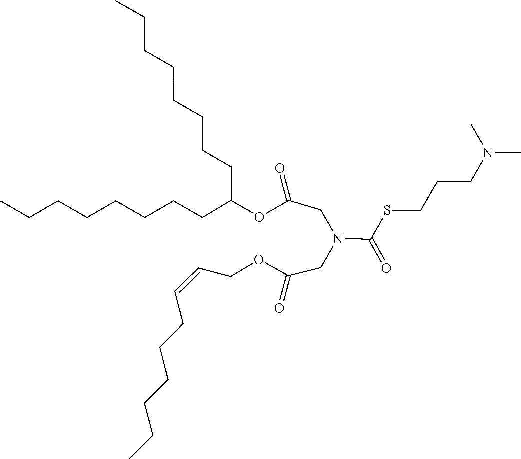 [SMILES: CCCCCC/C=C\COC(=O)CN(CC(=O)OC(CCCCCCCC)CCCCCCCC)C(=O)SCCCN(C)C]

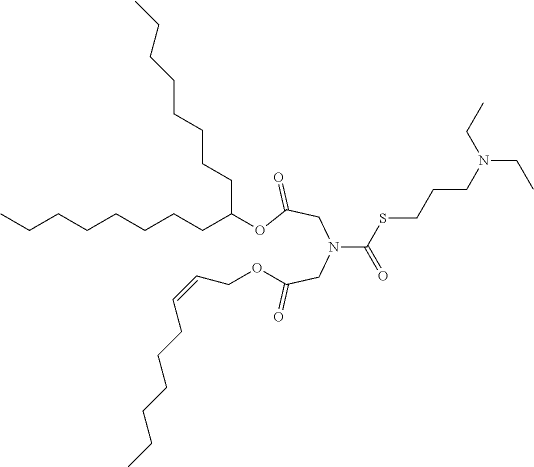 [SMILES: CCCCCC/C=C\COC(=O)CN(CC(=O)OC(CCCCCCCC)CCCCCCCC)C(=O)SCCCN(CC)CC]

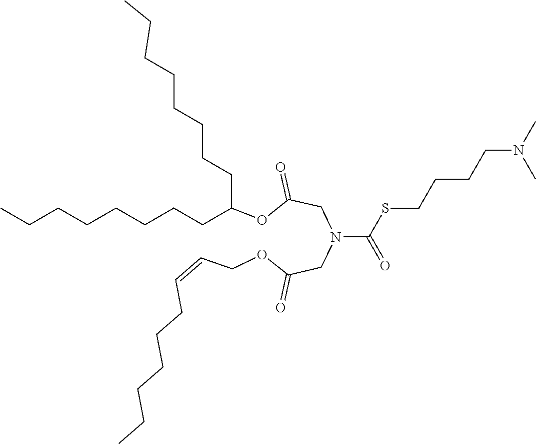 [SMILES: CCCCCC/C=C\COC(=O)CN(CC(=O)OC(CCCCCCCC)CCCCCCCC)C(=O)SCCCCN(C)C]

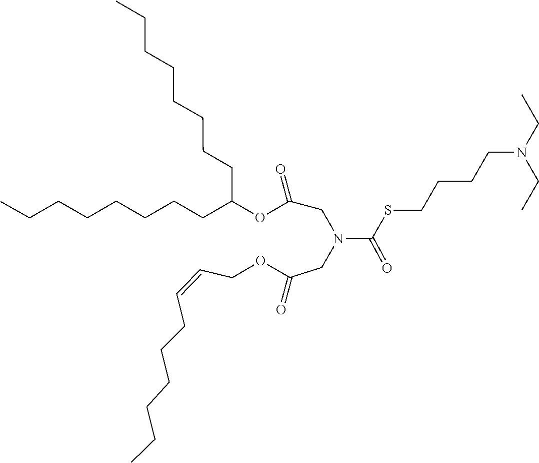 [SMILES: CCCCCC/C=C\COC(=O)CN(CC(=O)OC(CCCCCCCC)CCCCCCCC)C(=O)SCCCCN(CC)CC]

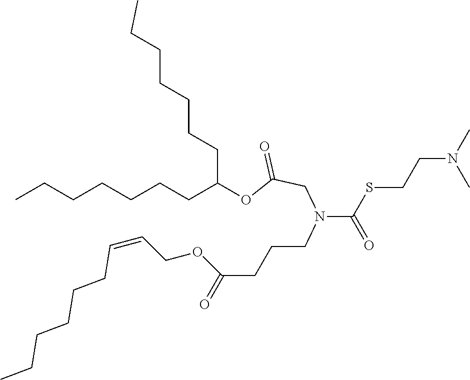 [SMILES: CCCCCC/C=C\COC(=O)CCCN(CC(=O)OC(CCCCCCC)CCCCCCC)C(=O)SCCN(C)C]

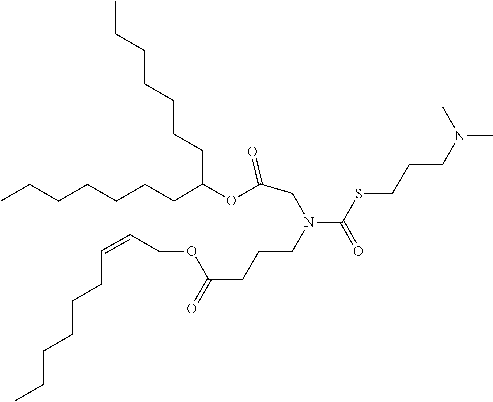 [SMILES: CCCCCC/C=C\COC(=O)CCCN(CC(=O)OC(CCCCCCC)CCCCCCC)C(=O)SCCCN(C)C]

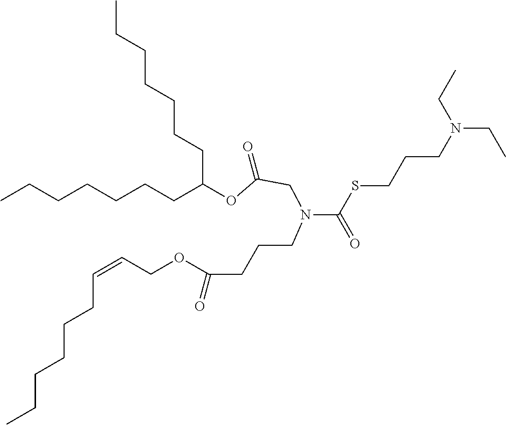 [SMILES: CCCCCC/C=C\COC(=O)CCCN(CC(=O)OC(CCCCCCC)CCCCCCC)C(=O)SCCCN(CC)CC]

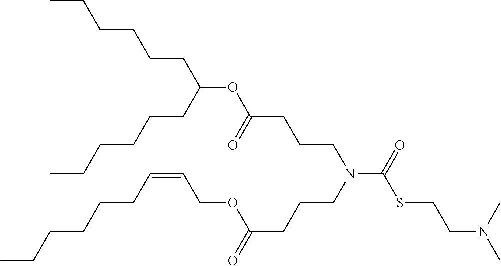 [SMILES: CCCCCC/C=C\COC(=O)CCCN(CCCC(=O)OC(CCCCCC)CCCCCC)C(=O)SCCN(C)C]

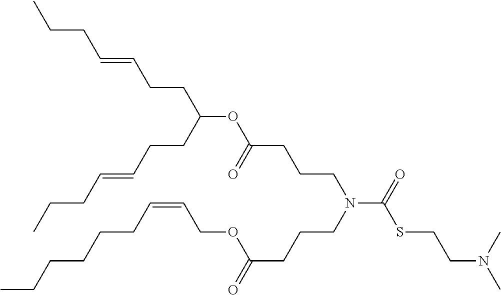 [SMILES: CCC/C=C/CCC(CC/C=C/CCC)OC(=O)CCCN(CCCC(=O)OC/C=C\CCCCCC)C(=O)SCCN(C)C]

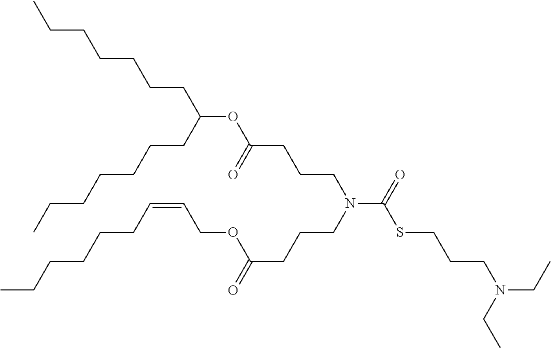 [SMILES: CCCCCC/C=C\COC(=O)CCCN(CCCC(=O)OC(CCCCCCC)CCCCCCC)C(=O)SCCCN(CC)CC]

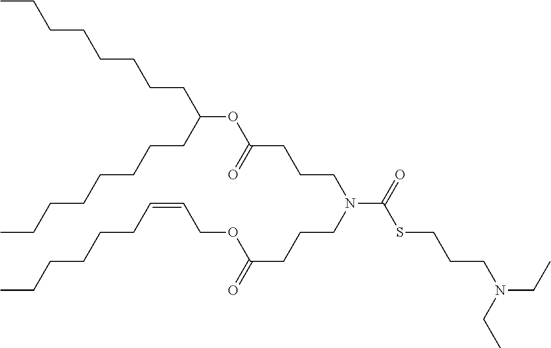 [SMILES: CCCCCC/C=C\COC(=O)CCCN(CCCC(=O)OC(CCCCCCCC)CCCCCCCC)C(=O)SCCCN(CC)CC]

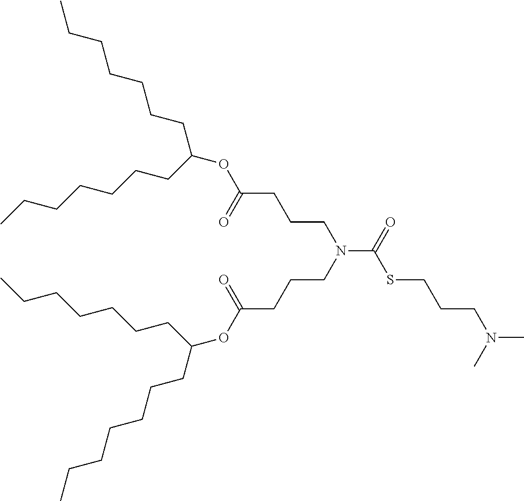 [SMILES: CCCCCCCC(CCCCCCC)OC(=O)CCCN(CCCC(=O)OC(CCCCCCC)CCCCCCC)C(=O)SCCCN(C)C]

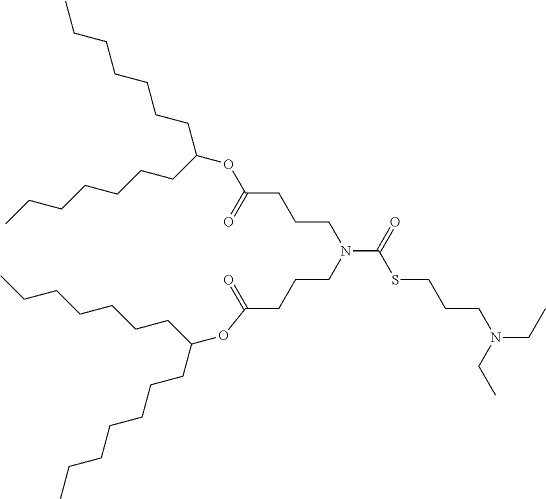 [SMILES: CCCCCCCC(CCCCCCC)OC(=O)CCCN(CCCC(=O)OC(CCCCCCC)CCCCCCC)C(=O)SCCCN(CC)CC]

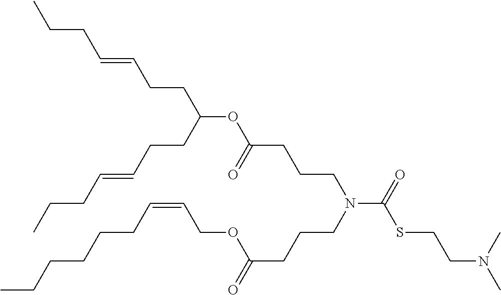 [SMILES: CCC/C=C/CCC(CC/C=C/CCC)OC(=O)CCCN(CCCC(=O)OC/C=C\CCCCCC)C(=O)SCCN(C)C]

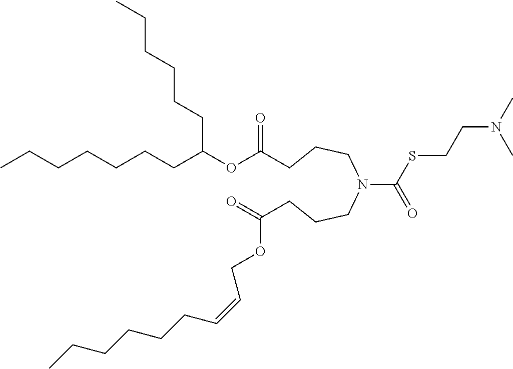 [SMILES: CCCCCC/C=C\COC(=O)CCCN(CCCC(=O)OC(CCCCCC)CCCCCCC)C(=O)SCCN(C)C]

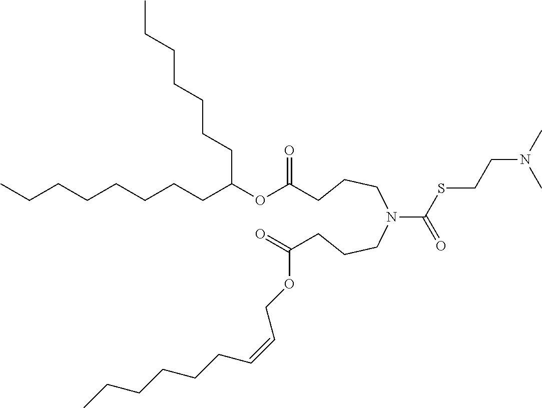 [SMILES: CCCCCC/C=C\COC(=O)CCCN(CCCC(=O)OC(CCCCCCC)CCCCCCCC)C(=O)SCCN(C)C]

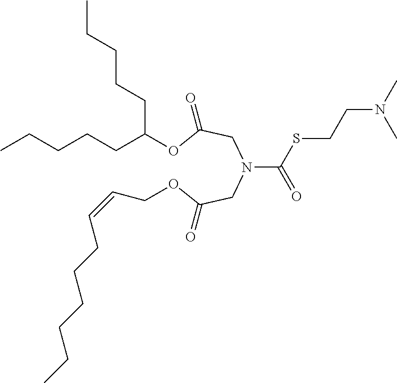 [SMILES: CCCCCC/C=C\COC(=O)CN(CC(=O)OC(CCCCC)CCCCC)C(=O)SCCN(C)C]

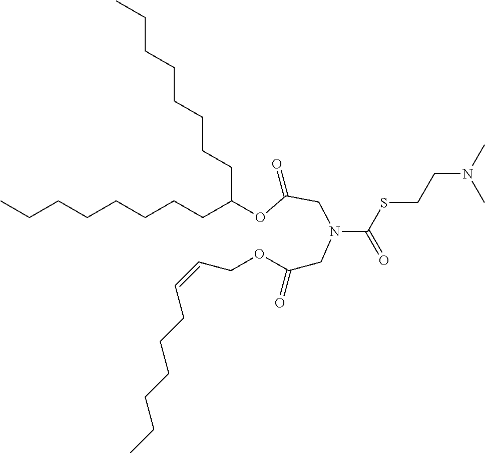 [SMILES: CCCCCC/C=C\COC(=O)CN(CC(=O)OC(CCCCCCCC)CCCCCCCC)C(=O)SCCN(C)C]

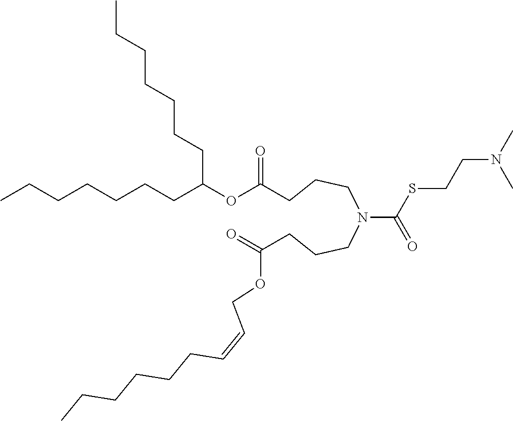 [SMILES: CCCCCC/C=C\COC(=O)CCCN(CCCC(=O)OC(CCCCCCC)CCCCCCC)C(=O)SCCN(C)C]

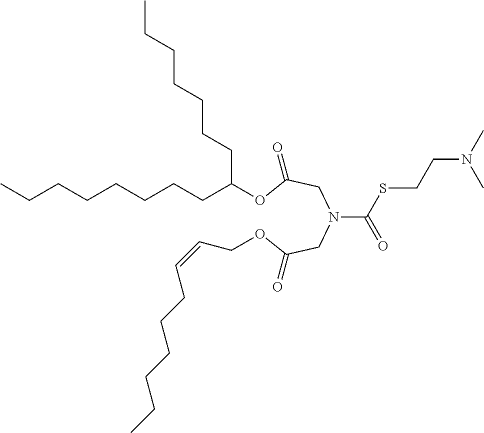 [SMILES: CCCCCC/C=C\COC(=O)CN(CC(=O)OC(CCCCCCC)CCCCCCCC)C(=O)SCCN(C)C]

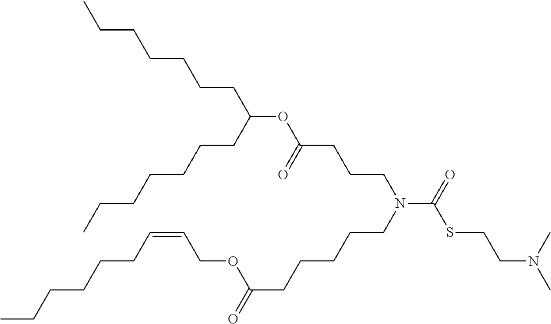 [SMILES: CCCCCC/C=C\COC(=O)CCCCCN(CCCC(=O)OC(CCCCCCC)CCCCCCC)C(=O)SCCN(C)C]

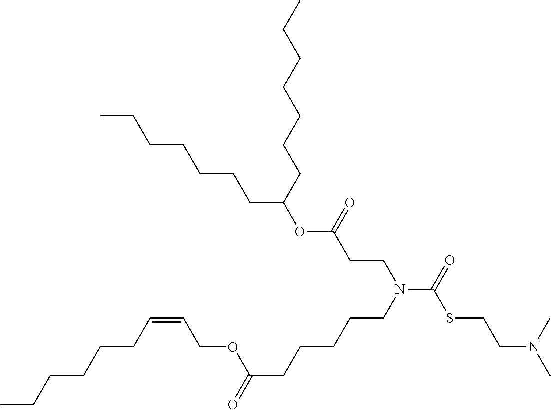 [SMILES: CCCCCC/C=C\COC(=O)CCCCCN(CCC(=O)OC(CCCCCCC)CCCCCCC)C(=O)SCCN(C)C]

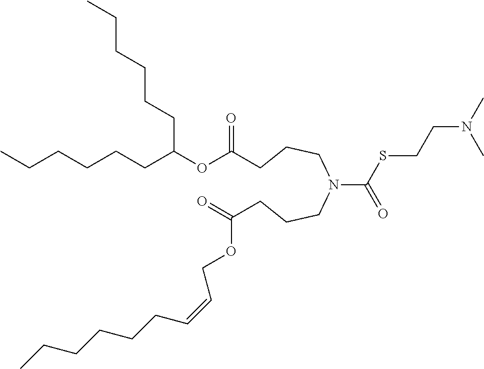 [SMILES: CCCCCC/C=C\COC(=O)CCCN(CCCC(=O)OC(CCCCCC)CCCCCC)C(=O)SCCN(C)C]

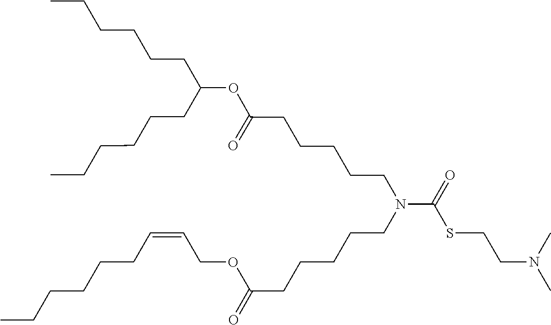 [SMILES: CCCCCC/C=C\COC(=O)CCCCCN(CCCCCC(=O)OC(CCCCCC)CCCCCC)C(=O)SCCN(C)C]

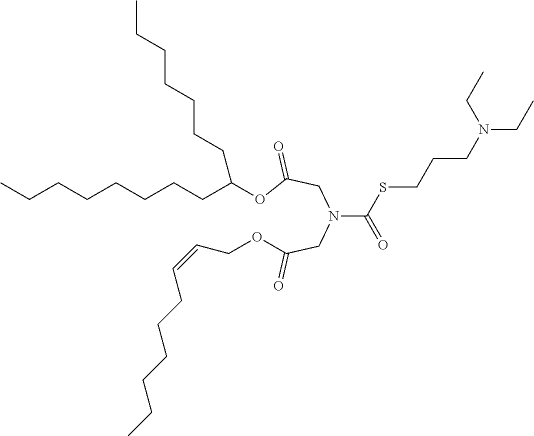 [SMILES: CCCCCC/C=C\COC(=O)CN(CC(=O)OC(CCCCCCC)CCCCCCCC)C(=O)SCCCN(CC)CC]

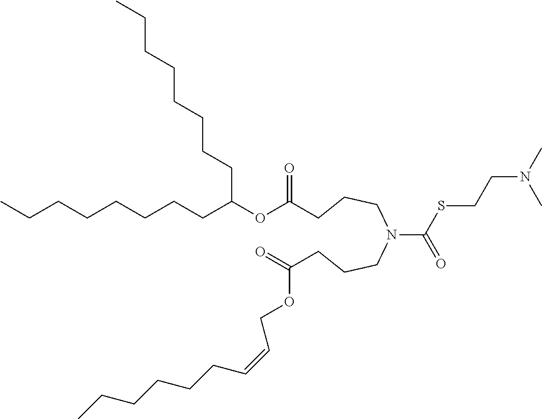 [SMILES: CCCCCC/C=C\COC(=O)CCCN(CCCC(=O)OC(CCCCCCCC)CCCCCCCC)C(=O)SCCN(C)C]

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated alkyl chains are more easily sized, particularly when the complexes must be sized below about 0.3 microns, for purposes of filter sterilization. Amino lipids containing unsaturated fatty acids with carbon chain lengths in the range of C₁₄ to C₂₂ may be used. Other scaffolds can also be used to separate the amino group and the fatty acid or fatty alkyl portion of the amino lipid.

In some embodiments, cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

Depending on the intended use of the lipid formulation, the proportions of the components can be varied, and the delivery efficiency of a particular formulation can be measured using assays known in the art.

In some embodiments, based on the total amount of lipids in the lipid nanoparticle, the lipid nanoparticle comprises about 5 mol % to 15 mol % of a combined amount of the compound of Formula I of the present disclosure and the helper lipid, about 30 mol % to about 70 mol % of the ionizable cationic lipid, about 20 mol % to about 60 mol % of cholesterol, and about 0.5 mol % to about 5 mol % of a PEG-lipid conjugate

In some embodiments, based on the total amount of lipids in the lipid nanoparticle, the lipid nanoparticle comprises about 5 mol % to 10 mol % of a combined amount of the compound of Formula I of the present disclosure and the helper lipid, about 40 mol % to about 60 mol % of the ionizable cationic lipid, about 30 mol % to about 50 mol % of cholesterol, and about 0.5 mol % to about 3 mol % of a PEG-lipid conjugate.

In some embodiments, based on the total amount of lipids in the lipid nanoparticle, the lipid nanoparticle comprises about 5 mol % to 12 mol % of a combined amount of the compound of Formula I of the present disclosure and the helper lipid, about 45 mol % to about 55 mol % of the ionizable cationic lipid, about 35 mol % to about 45 mol % of cholesterol, and about 0.5 mol % to about 3 mol % of a PEG-lipid conjugate.

The percentage of helper lipid present in the lipid formulation is a target amount, and the actual amount of helper lipid present in the formulation may vary dependent on the specific formulation, for example, by +5 mol %.

In some embodiments, lipid nanoparticle has a total lipid:nucleic acid weight ratio of about 50:1 to about 10:1. In some embodiments, the lipid nanoparticle has a total lipid:nucleic acid weight ratio of about 40:1 to about 20:1. In some embodiments, the lipid nanoparticle has a total lipid:nucleic acid weight ratio of about 35:1 to about 25:1. In some embodiments, the lipid nanoparticle has a total lipid:nucleic acid weight ratio of about 32:1 to about 28:1. In some embodiments, the lipid nanoparticle has a total lipid:nucleic acid weight ratio of about 31:1 to about 29:1.

The pharmaceutical composition can be formulated for therapeutic use, in which case other components such as pharmaceutically acceptable buffers and salts, and stabilizers such as cryoprotectants, can be included.

In some embodiments, the dosage form of the pharmaceutical compositions described herein can be a liquid suspension of nucleic acid-lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In some embodiments, the suspension is frozen during storage and thawed prior to administration. In some embodiments, the suspension is frozen at a temperature below about −70° C. In some embodiments, the suspension is diluted with sterile water prior to inhalable administration. In some embodiments, an inhalable administration comprises diluting the suspension with about 1 volume to about 4 volumes of sterile water. In some embodiments, a lyophilized nucleic acid-lipid nanoparticle formulation can be resuspended in a buffer as described herein.

In some embodiments, the pharmaceutical composition comprises a HEPES buffer at a pH of about 7.4. In some embodiments, the HEPES buffer is at a concentration of about 7 mg/mL to about 15 mg/mL.

In some embodiments, the pharmaceutical composition further comprises about 2.0 mg/mL to about 4.0 mg/mL of NaCl.

In some embodiments, the pharmaceutical composition further comprises one or more cryoprotectants. In some embodiments, the one or more cryoprotectants are selected from sucrose, glycerol, or a combination of sucrose and glycerol. In some embodiments, the cryoprotectant is sucrose.

In some embodiments, the pharmaceutical composition comprises a combination of sucrose at a concentration of about 70 mg/mL to about 110 mg/mL and glycerol at a concentration of about 50 mg/mL to about 70 mg/mL.

The compounds disclosed herein can be formulated with conventional carriers and excipients. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986). Excipients can include, for example, ascorbic acid and other antioxidants, chelating agents such as EDTA, carbohydrates such as dextran, hydroxyalkylcellulose, hydroxyalkylmethylcellulose, stearic acid, and combinations thereof. In some embodiments, the formulation is basic. In some embodiments, the formulation is acidic. In some embodiments, the formulation has a neutral pH. In some embodiments, the pH of the formulations is from 2 to 11 (e.g., 2, 3, 4, 5, 6, 7, 8, 9, 10, 11, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, or 9-11).

In some embodiments, the compounds disclosed herein are administered alone. In some embodiments, the compounds disclosed herein are administered in pharmaceutical compositions. In some embodiments, the pharmaceutical compositions are for veterinary use. In some embodiments, the pharmaceutical compositions are for human use. In some embodiments, the pharmaceutical compositions disclosed herein include at least one additional therapeutic agent. In some embodiments, the pharmaceutical compositions disclosed herein include one or more additional therapeutic agent. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

Pharmaceutical compositions disclosed herein can be in any form suitable for the intended method of administration. The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses. The pharmaceutical compositions disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes at least accessory ingredients. In general, the formulations can be prepared by uniformly and intimately bringing into association the active ingredient with liquid carriers or finely divided solid carriers or both, and then, if necessary, shaping the product.

The pharmaceutical compositions can be in the form of a sterile injectable or intravenous preparation, such as a sterile injectable aqueous or oleaginous suspension. This suspension can be formulated according to the known art using those suitable dispersing or wetting agents and suspending agents which have been mentioned above. The sterile injectable or intravenous preparation can also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution and isotonic sodium chloride solution. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. Among the acceptable vehicles and solvents that can be employed include, but are not limited to, water, Ringer's solution isotonic sodium chloride solution, and hypertonic sodium chloride solution.

The amount of active ingredient that can be combined with the carrier material to produce a single dosage form will vary depending upon the host treated and the particular mode of administration. For example, a time-release formulation intended for oral administration to humans can contain approximately 1 mg to 2000 mg of active material compounded with an appropriate and convenient amount of carrier material, which can vary from 5% to 95% of the total formulations (weight:weight). For example, a time-release formulation intended for oral administration to humans can contain approximately 1 mg to 1000 mg of active material compounded with an appropriate and convenient amount of carrier material, which can vary from 5% to 95% of the total formulations (weight:weight). The pharmaceutical composition can be prepared to provide easily measurable amounts for administration. For example, an aqueous solution intended for intravenous infusion can contain from 3 μg to 500 μg of the active ingredient per milliliter of solution in order that infusion of a suitable volume at a rate of 30 mL/hr can occur.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier, for example water for injection, immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with a primary DNA construct, or mRNA (e.g., for transplantation into a subject), hyaluronidase, nanoparticle mimics and combinations thereof.

In some embodiments, the formulations described herein can include one or more excipients, each in an amount that together increases the stability of the nucleic acid in the lipid formulation, increases cell transfection by the nucleic acid (e.g., mRNA or siRNA), increases the expression of an encoded protein, and/or alters the release profile of the encoded protein, or increases knockdown of a target native nucleic acid. Further, a nucleic acid may be formulated using self-assembled nucleic acid nanoparticles.

A dosage form of the composition of this disclosure can be solid, which can be reconstituted in a liquid prior to administration. The solid can be administered as a powder. The solid can be in the form of a capsule, tablet, or gel.

To formulate compositions for pulmonary delivery within the present disclosure, the nucleic acid-lipid formulation can be combined with various pharmaceutically acceptable additives, as well as a base or carrier for dispersion of the nucleic acid-lipid formulation(s). Examples of additives include pH control agents such as arginine, sodium hydroxide, glycine, hydrochloric acid, citric acid, and mixtures thereof. Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of ⅓ to 3, more typically ½ to 2, and most often ¾ to 1.7.

The nucleic acid-lipid formulation may be dispersed in a base or vehicle, which may comprise a hydrophilic compound having a capacity to disperse the nucleic acid-lipid formulation and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the nucleic acid-lipid formulation.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

It should be understood that in addition to the ingredients particularly mentioned above the formulations can include other agents conventional in the art having regard to the type of formulation in question.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical compositions containing as active ingredient one or more of the compounds (“controlled release formulations”) in which the release of the active ingredient can be controlled and regulated to allow less frequency dosing or to improve the pharmacokinetic or toxicity profile of a given active ingredient.

### V. Kits

Also provided herein are kits that includes a compound of the present disclosure or a pharmaceutically acceptable salt thereof, a pharmaceutical composition of the present disclosure, a lipid composition of the present disclosure, or a lipid nanoparticle of the present disclosure. In some embodiments the kits described herein can comprise a label and/or instructions for use of the compound, the composition, or the lipid nanoparticle in the treatment of a disease or condition in a subject (e.g., human) in need thereof. In some embodiments, the disease or condition is cancer, or immune diseases. In some embodiments, the disease or condition is associated with abnormal expression of a gene such as over expression of a gene.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof, and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more of a compound of the present disclosure or a pharmaceutically acceptable salt thereof, a pharmaceutical composition of the present disclosure, a lipid composition of the present disclosure, or a lipid nanoparticle of the present disclosure are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. Preferably, the administration is by parenteral such as intravenously or intramuscularly.

The compounds of the present disclosure (also referred to herein as the active ingredients), can be administered by any route appropriate to the condition to be treated.

Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the route may vary with for example the condition of the recipient. An advantage of certain compounds disclosed herein is that they are orally bioavailable and can be dosed orally.

A compound, composition or lipid nanoparticle of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound, composition or lipid nanoparticle is administered on a daily or intermittent schedule for the duration of the individual's life. In some instances, when the compound, composition or lipid nanoparticle of the present disclosure is used as a prophylaxis, for example as a vaccine, the compound, composition or lipid nanoparticle may be administered only once, twice, or three times.

The dosage or dosing frequency of a compound, composition or lipid nanoparticle of the present disclosure may be adjusted over the course of the treatment, based on the judgment of the administering physician.

The compound may be administered to an individual (e.g., a human) in an effective amount. In some embodiments, the compound is administered once daily.

According to the present disclosure, a therapeutically effective dose of the provided composition, when administered regularly, may result in an increased nucleic acid activity level in a subject as compared to a baseline activity level before treatment. Typically, the activity level is measured in a biological sample obtained from the subject such as blood, plasma or serum, urine, or solid tissue extracts. The baseline level can be measured immediately before treatment. In some embodiments, administering a pharmaceutical composition described herein results in an increased nucleic acid activity level in a biological sample (e.g., plasma/serum or lung epithelial swab) by at least about 10%, 20%, 30%, 40%, 50%, 60%, 70%, 80%, 90%, or 95% as compared to a baseline level before treatment. In some embodiments, administering the provided composition results in an increased nucleic acid activity level in a biological sample (e.g., plasma/serum or lung epithelial swab) by at least about 10%, 20%, 30%, 40%, 50%, 60%, 70%, 80%, 90%, or 95% as compared to a baseline level before treatment for at least about 24 hours, at least about 48 hours, at least about 72 hours, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days.

### VII. Methods of Use

In some embodiments, the present disclosure includes a method treating and/or preventing a disease in a subject in need thereof, comprising administering a therapeutically effective amount to the subject, the compound, the pharmaceutical composition, the lipid composition, or the lipid nanoparticle of the present disclosure.

In some embodiments, the present application includes a method of expressing a protein or polypeptide in a target cell, comprising contacting the target cell with the compound, the pharmaceutical composition, the lipid composition, or the lipid nanoparticle of the present disclosure.

In some embodiments, the present application includes a method of delivering a nucleic acid to a subject in need thereof, comprising encapsulating a therapeutically effective amount of the nucleic acid in the compound, the pharmaceutical composition, the lipid composition, or the lipid nanoparticle of the present disclosure, and administering the compound, the pharmaceutical composition, the lipid composition, or the lipid nanoparticle of the present disclosure to the subject.

In some embodiments, the protein or polypeptide is an antigen, and expression of the antigen elicits an in vivo immunogenic response.

The compound, the pharmaceutical composition, the lipid composition, or the lipid nanoparticle of the present disclosure can be administered by any suitable methods known in the field. In some embodiments, the compound, the pharmaceutical composition, the lipid composition, or the lipid nanoparticle of the present disclosure is administered intravenously or intramuscularly. It is understood that for intravenous or intramuscular administration, the compound, the pharmaceutical composition, the lipid composition, or the lipid nanoparticle of the present disclosure can be suitable formulated as described herein or as understood by conventional means known in the field.

In some embodiments, the lipid formulations may be administered in a local rather than systemic manner. Local delivery can be affected in various ways, depending on the tissue to be targeted. For example, aerosols containing compositions of the present disclosure can be inhaled (for nasal, tracheal, or bronchial delivery).

In some embodiments, the composition or the lipid nanoparticle of the present disclosure further comprises a nucleic acid. The lipid composition or lipid nanoparticle of the present disclosure can be used as a delivery vehicle for delivering the nucleic acid to a target cell or tissue.

In some embodiments, the lipid composition or lipid nanoparticle of the present disclosure may be administered to any desired tissue. In some embodiments, the nucleic acid delivered by a lipid formulation or composition of the present disclosure is active in the tissue in which the lipid formulation and/or composition was administered. In some embodiments, the nucleic acid is active in a tissue different from the tissue in which the lipid formulation or composition was administered. Example tissues in which the nucleic acid may be delivered include, but are not limited to the lung, trachea, and/or nasal passages, muscle, liver, eye, or the central nervous system.

In some embodiments, there are provided a methods of treating a disease or disorder in a mammalian subject. A therapeutically effective amount of a compound, a pharmaceutical composition, a lipid composition or a lipid nanoparticle of the present disclosure may be administered to a subject having a disease or disorder associated with expression or overexpression of a gene that can be reduced, decreased, downregulated, or silenced by the composition. The lipid compositions and lipid nanoparticles described herein can be used in a methods for treating cancer or inflammatory disease. The disease may be one selected from the group consisting of central nervous system disorders, peripheral nervous system disorders, muscle atrophies, muscle dystrophies, immune disorder, cancer, renal disease, fibrotic disease, genetic abnormality, inflammation, and cardiovascular disorder.

### VIII. Mechanism of Action for Cellular Update of Lipid Composition/Nanoparticle

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Specifically, in the case of a nucleic acid-lipid formulations described herein, the lipid formulation enters cells through receptor mediated endocytosis. Prior to endocytosis, functionalized ligands such as a the lipid conjugate of the disclosure at the surface of the lipid delivery vehicle can be shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For mRNA or self-replicating RNA payloads, the cell's own internal translation processes will then translate the RNA into the encoded protein. The encoded protein can further undergo post-translational processing, including transportation to a targeted organelle or location within the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, ion exchange chromatography, and supercritical fluid chromatography (SFC). Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4th ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product. Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or diastereomerically pure.

Compounds disclosed herein can be prepared from commercially available reagents using the synthetic methods and reaction schemes described herein, or using other reagents and conventional methods known to persons of ordinary skill in the art. For instance, representative syntheses of compounds of the present disclosure are described in the schemes below, and the particular examples that follow.

### X. Lipid Composition/Nanoparticle Preparation

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual asymmetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### EXAMPLES

### I. Abbreviations

Certain abbreviations and acronyms are used in describing experimental details. Although most of these would be understood by one skilled in the art, Table 1 contains a list of many of these abbreviations and acronyms.

TABLE 1
List of Abbreviations and Acronyms

| Abbreviation | Meaning |
|---|---|
| ° C. | degree(s) Celsius |
| μg or ug | microgram(s) |
| μL or uL | microliter(s) |
| μm or um | micron(s) |
| μmol or umol | micromole(s) |
| aq | Aqueous |
| Boc | tert-butoxycarbonyl |
| br s | broad singlet |
| Cbz | Benzyloxycarbonyl |
| d | Doublet |
| DCM | Dichloromethane |
| dd | doublet of doublets |
| ddd | doublet of doublet of doublets |
| ddt | doublet of doublet of triplets |
| DIPEA | N,N-diisopropylethylamine |
| DMAP | 4-dimethylaminopyridine |
| DMF | Dimethylformamide |
| DMP | Dess-Martin periodinane |
| DMSO | dimethyl sulfoxide |
| dt | doublet of triplets |
| Et | Ethyl |
| EtOAc | ethyl acetate |
| g | gram(s) |
| h | hour(s) |
| HATU | hexafluorophosphate azabenzotriazole tetramethyl uronium |
| HPLC | high-performance liquid chromatography |
| Hz | Hertz |
| ⁱPr | isopropyl |
| IPA or iPrOH | isopropyl alcohol |
| J | coupling constant |
| KHMDS | potassium bis(trimethylsilyl)amide |
| KOAc | potassium acetate |
| LAH | lithium aluminum hydride |
| LCMS | liquid chromatography mass spectrometry |
| LDA | lithium diisopropylamide |
| LiHMDS | lithium bis(trimethylsilyl)amide |
| m | multiplet |
| M | Molarity |
| Me | Methyl |
| MeCN | acetonitrile |
| MeOH | methanol |
| mg | milligram(s) |
| MHz | megahertz |
| min | minute(s) |
| mL | milliliter(s) |
| mm | millimeter(s) |
| mmol | millimole(s) |
| MOMO or | methoxymethyl |
| OMOM | |
| NaOMe | sodium methoxide |
| n-BuLi | n-butyllithium |
| NMR | nuclear magnetic resonance |
| OSEM | 2-(trimethylsilyl)ethoxymethoxy |
| OTf | trifluoromethanesulfonate |
| Piv | Pivaloyl |
| qd | quartet of doublets |
| rt | room temperature |
| s | Singlet |
| SFC | supercritical fluid chromatography |
| t | Triplet |
| TBAF | tetrabutylammonium fluoride |
| TBS | tert-butyldimethylsilyl |
| TBSCl | tert-Butyldimethylsilyl chloride |
| ^{t}Bu | tert-butyl |
| td | triplet of doublets |
| TES | triethylsilane |
| TF₂O or Tf₂O | trifluoromethanesulfonic anhydride |
| TFA | trifluoroacetic acid |
| THF | tetrahydrofuran |
| TIPS | triisopropylsilyl |
| TMS | trimethylsilyl |
| tt | triplet of triplets |
| v/v | volume/volume |
| wt | Weight |
| δ | parts per million referenced to residual non-deuterated solvent peak |

### II. Compounds

### Example 1 Synthesis of Compound I-1

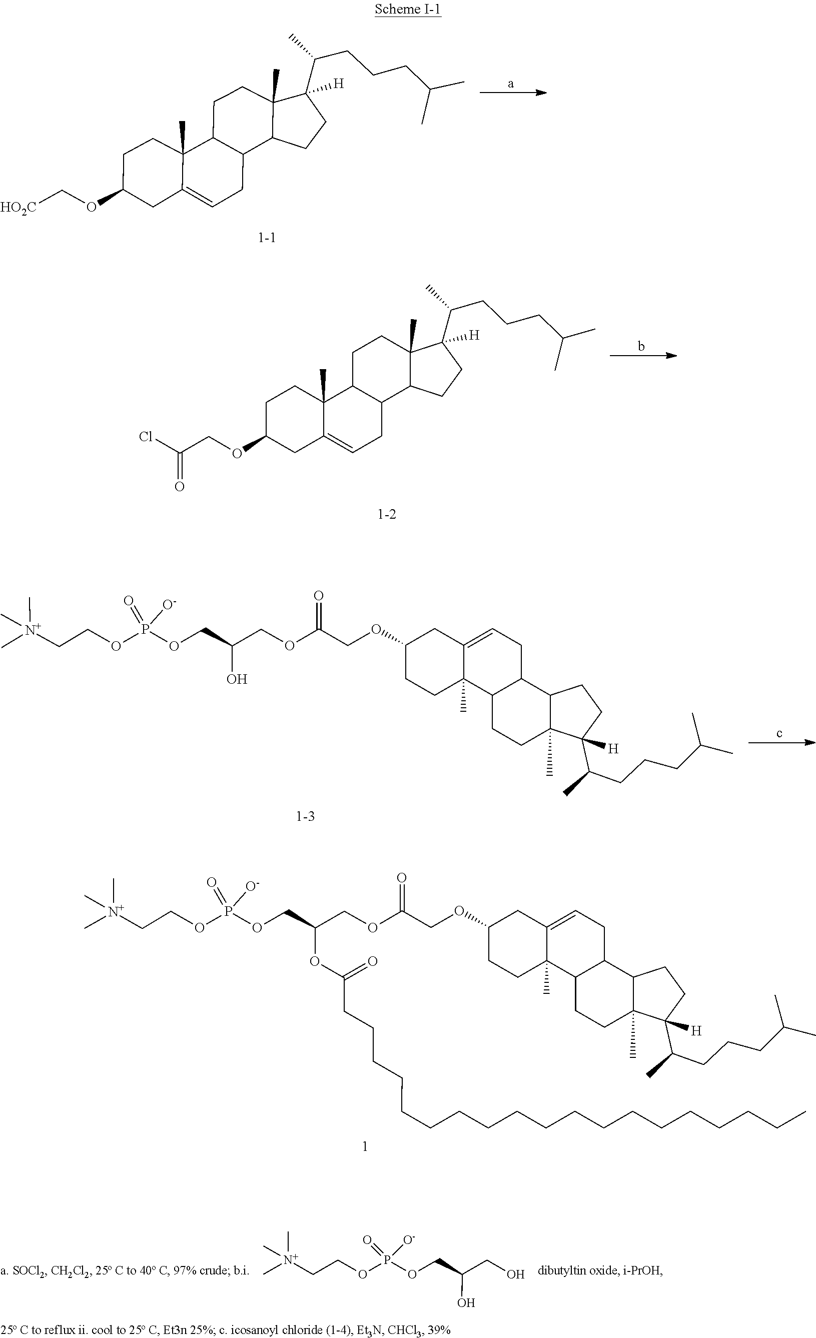

### 2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetyl chloride (1-2)

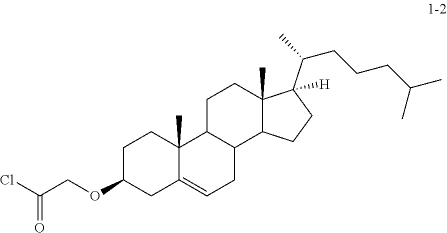 [SMILES: CC(C)CCC[C@@H](C)[C@H]1CCC2C3CC=C4C[C@@H](OCC(=O)Cl)CC[C@]4(C)C3CC[C@@]21C]

2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetic acid (1-1, 25.0 g, 56.2 mmol) (Chorny, M; Alferiev, I. S.; Fishbein, L; Tengood, J. E.; Folchman-Wagner, Z.; Forbes, S. P.; Levy, R. J. Pharm. Res. 2012, 29, 1232-1241) was dissolved in CH₂Cl₂ (125 mL) at room temperature under nitrogen. To this solution was added thionyl chloride (125 mL, 205.0 g, 1.72 mol) dropwise over a period of 20 minutes. After the addition was complete the mixture was stirred for 2 hours then the mixture was concentrated in vacuo to give crude 1-2 (25.3 g, 54.6 mmol, 97% crude yield) as a white solid.

¹H-NMR (300 MHz, CDCl₃): δ 5.38 (m, 1H), 4.46 (s, 2H), 3.31 (m, 1H), 2.31 (m, 2H), 1.76-2.08 (5H), 0.65-1.60 (33H), 0.69 (s, 3H).

### (2R)-3-(2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetoxy)-2-hydroxypropyl (2-(trimethylammonio)ethyl) phosphate (1-3)

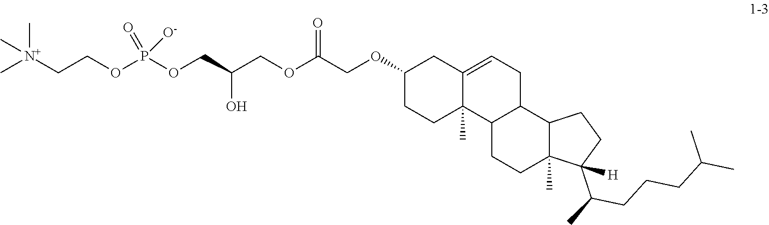 [SMILES: CC(C)CCC[C@@H](C)[C@H]1CCC2C3CC=C4C[C@@H](OCC(=O)OC[C@@H](O)COP(=O)([O-])OCC[N+](C)(C)C)CC[C@]4(C)C3CC[C@@]21C]

A mixture of (R)-2,3-dihydroxypropyl (2-(trimethylammonio)ethyl) phosphate (sn-glycero-3-phosphocholine, 5.00 g, 19.41 mmol) and dibutyltin oxide (5.08 g, 20.41 mmol) was dissolved in isopropanol (200 mL), under nitrogen. After solution was achieved the mixture was heated under reflux for 2 hours, then was cooled to room temperature. To this cooled solution was added Et₃N (2.36 g, 23.35 mmol), in one portion, followed by the addition of 1-2 (10.8 g, assumed 23.31 mmol). The mixture was allowed to stir for 3 hours at room temperature then was filtered and the filtrate was concentrated in vacuo to give the crude reaction mixture. The crude mixture was dissolved in CH₂Cl₂ (150 mL) and silica gel (30 g, type: ZCX-100-200 mesh) was added. The solvent was removed in vacuo and the silica gel containing adsorbed 1-3 was loaded atop a silica gel column (150 g, type: ZCX-100-200 mesh) which was eluted with a gradient of CH₂Cl₂:MeOH from 100:0 to 90:10. Qualified fractions were pooled and concentrated in vacuo to afford 1-3 (3.30 g, 4.83 mmol, 25%) as a white solid.

LC-MS: (+ mode) RT 2.251 min, Calcd. For C₃₇H₆₆NO₈P 683.45, Found 684.50 (M+H⁺); ¹H-NMR (300 MHz, CD₃OD): d 5.38 (m, 1), 4.12-4.38 (6H), 3.85-4.09 (3H), 3.62-3.69 (2H), 3.23 (s, 9H), 1.81-2.40 (8H), 0.87-1.69 (34H), 0.73 (s, 3H).

### Icosanoyl Chloride (1-4)

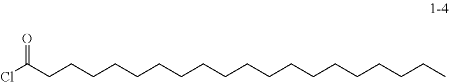 [SMILES: CCCCCCCCCCCCCCCCCCCC(=O)Cl]

Icosanoic acid (8.00 g, 25.60 mmol) was dissolved in CH₂Cl₂ (50 mL) under nitrogen. To this solution was added thionyl chloride (40 mL, 65.6 g, 0.551 mol) dropwise over a period of 25 minutes. The mixture was allowed to stir for 30 minutes after the addition was complete, then was warmed to 70° C. and was stirred for 2 hours. The mixture was concentrated in vacuo to give crude icosanoyl chloride (8.40 g, 25.4 mmol, 99% crude yield) as a white solid. ¹H-NMR (400 MHz, CDCl₃): δ 2.90 (t, J=7.3 Hz, 2H), 1.74 (m, 2H), 1.20-1.42 (32H), 0.91 (t, J=6.7 Hz, 3H).

### (2R)-3-(2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetoxy)-2-(icosanoyloxy)propyl (2-(trimethylammonio)ethyl) phosphate (I-1)

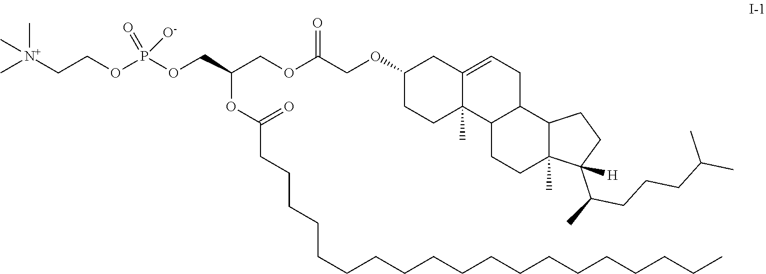 [SMILES: CCCCCCCCCCCCCCCCCCCC(=O)O[C@H](COC(=O)CO[C@H]1CC[C@@]2(C)C(=CCC3C2CC[C@@]2(C)C3CC[C@@H]2[C@H](C)CCCC(C)C)C1)COP(=O)([O-])OCC[N+](C)(C)C]

To a solution of 1-3 (3.30 g, 4.83 mmol) in CHCl₃ (75 mL), under nitrogen at room temperature, was added in order Et₃N (2.44 g, 24.15 mmol) and icosanoyl chloride (8.00 g, 24.2 mmol). The mixture was stirred at room temperature under nitrogen for 48 hours, then was concentrated in vacuo to give crude 1 that was purified by Prep-Chiral HPLC (column: CHIRALPAK IG 3×25 cm, 5 mm, 87475S901G0SCZ-WJ001); Mobile Phase A: hexanes with 0.1% Et₃N, Mobile Phase B: EtOH, gradient 15%-15% over 40 minutes. Qualified fractions were combined, concentrated in vacuo and the residue was dissolved in CHCl₃ (20 mL). The CHCl₃ solution of 1 was washed with water (3×20 mL) and concentrated in vacuo to provide 1 (0.60 g, 0.613 mmol, 12.7%) as a pale yellow, solid. SFC-MS: (+ mode) RT 3.13 min. Calcd. For C₅₇H₁₀₄NO₉P 977.74, Found 979.10 (M+H⁺); ¹H-NMR (400 MHz, CDCl₃): δ 5.25-5.40 (2H), 4.10-4.64 (6H), 4.00 (m, 1H), 3.84 (m, 2H), 3.46 (s, 9H), 3.26 (m, 1H), 2.92 (m, 1H), 2.20-2.42 (4H), 1.74-2.10 (5H), 0.85-1.60 (70H), 0.69 (s, 3H); ³¹P-NMR (400 MHz, CDCl₃): 16.01 ppm.

### Example 2 Synthesis of Compound I-2

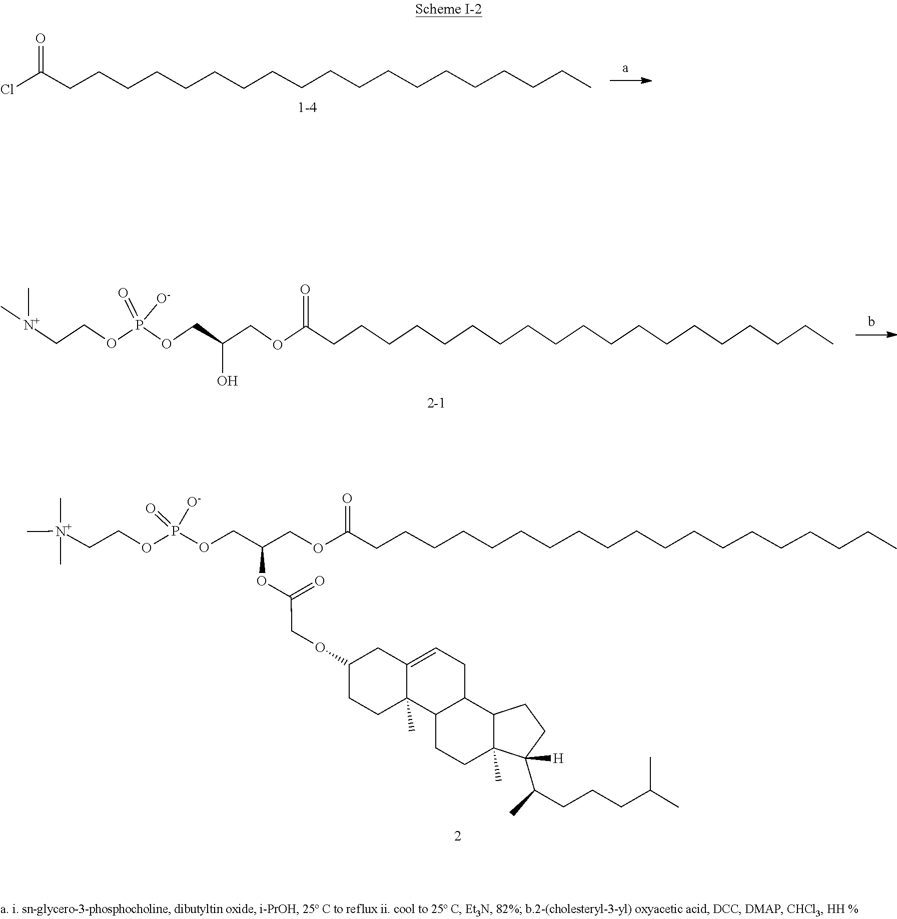

### (R)-2-hydroxy-3-(icosanoyloxy)propyl (2-(trimethylammonio)ethyl) phosphate (2-1)

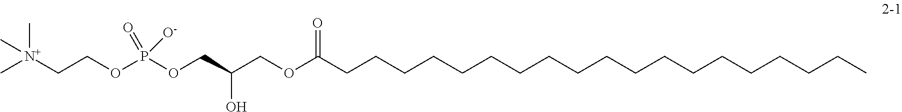 [SMILES: CCCCCCCCCCCCCCCCCCCC(=O)OC[C@@H](O)COP(=O)([O-])OCC[N+](C)(C)C]

A mixture of (R)-2,3-dihydroxypropyl (2-(trimethylammonio)ethyl) phosphate (sn-glycero-3-phosphocholine, 7.40 g, 28.76 mmol) and dibutyltin oxide (7.18 g, 28.85 mmol) was dissolved in isopropanol (300 mL), under nitrogen. After solution was achieved the mixture was heated under reflux for 4 hours, then was cooled to room temperature. To this cooled solution was added Et₃N (3.49 g, 34.53 mmol), in one portion, followed by the addition of 1-4 (10.50 g, assumed 31.72 mmol). The mixture was allowed to stir for 30 minutes at room temperature then was heated to 80° C. and was allowed to stir for 12 hours. The mixture was cooled to room temperature, then was cast into water (250 mL), filtered and the filtrate was concentrated in vacuo to give the crude reaction mixture. The crude mixture was dissolved in CH₂Cl₂ (250 mL) and silica gel (50 g, type: ZCX-100-200 mesh) was added. The solvent was removed in vacuo and the silica gel containing adsorbed 2-1 was loaded atop a silica gel column (300 g, type: ZCX-100-200 mesh) which was eluted with a gradient of CH₂Cl₂:MeOH from 100:0 to 90:10. Qualified fractions were pooled and concentrated in vacuo to afford 2-1 (13.00 g, 23.56 mmol, 82%) as a white solid. LC-MS (+ mode): RT 2.388 min, Calcd. for C₂₈H₅₈NO₇P 551.40, Found 552.45; ¹H-NMR (400 MHz, CD₃OD): d 4.37 (m, 2H), 4.14 (m, 2H), 3.80-4.02 (3H), 3.68 (m, 2H), 3.30 (s, 9H), 2.34 (m, 2H), 1.61 (m, 2H), 1.22-1.32 (33H), 0.90 (t, J=6.0 Hz, 3H).

### (2R)-2-(2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetoxy)-3-(icosanoyloxy)propyl (2-(trimethylammonio)ethyl) phosphate (I-2)

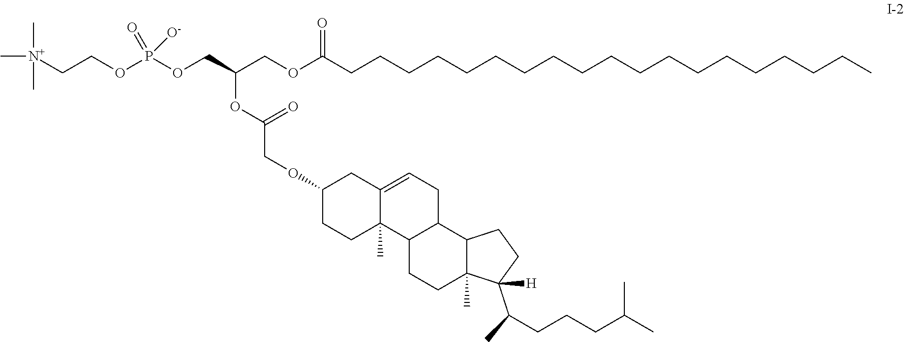 [SMILES: CCCCCCCCCCCCCCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1]

To a solution of 2-1 (4.59 g, 8.32 mmol) and 1-1 (4.40 g, 9.89 mmol) in CHCl₃ (75 mL), cooled in an ice-water bath under nitrogen, was successively added DCC (4.12 g, 19.96 mmol) and DMAP (0.40 g, 3.27 mmol). The mixture was stirred for 30 minutes at 0° C., warmed to room temperature and was allowed to stir for 12 hours. The mixture was concentrated in vacuo and the residue was dissolved in CH₂Cl₂ (100 mL). The solvent was removed in vacuo and the silica gel containing adsorbed 2 was loaded atop a silica gel column (400 g, type: ZCX-100-200 mesh) which was eluted with a gradient of CH₂Cl₂:MeOH from 100:0 to 80:20. Qualified fractions were pooled and concentrated in vacuo to afford 2 (4.50 g, 4.60 mmol, 55%) as a white solid.

LC-MS (+ mode): RT 3.97 min, Calcd. for C₅₇H₁₀₄NO₉P 977.74, Found 979.1 (M+H⁺); ¹H-NMR (300 MHz, CDCl₃): δ 5.30-5.50 (2H), 4.30-4.50 (3H), 4.10-4.29 (3H), 4.03 (m, 2H), 3.84 (m, 2H), 3.39 (s, 9H), 2.20-2.46 (4H), 1.80-2.10 (5H), 0.80-1.70 (71H), 0.66 (s, 3H).

### Example 3 Synthesis of Compound I-3

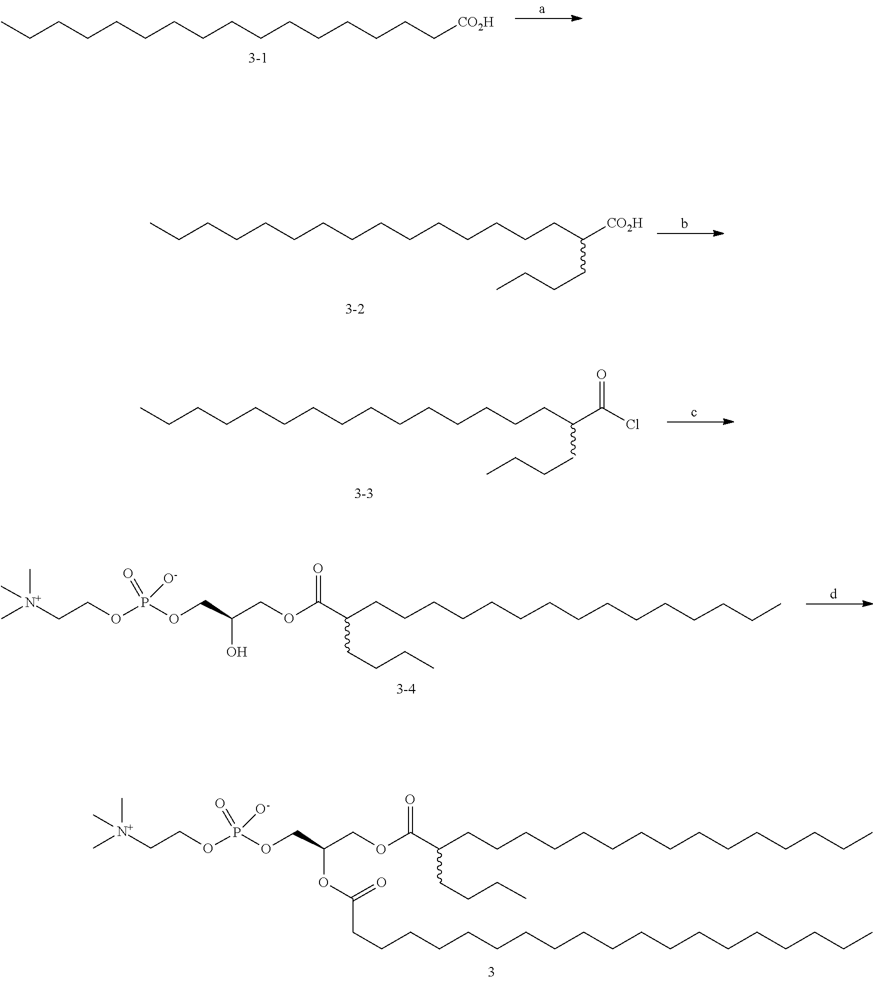

a. i. LDA, THF, HMPA, −20° C.; ii. n-BuBr, −20° C. to 25° C. 37%; b. SOCl₂, CH₂Cl₂, 25° C. to 40° C., 89% crude; c. i.sn-glycero-3-phosphocholine, dibutyltin oxide, i-PrOH, 25° C. to reflux ii. cool to 25° C., Et₃N 70%; d. icosanoic acid, DCC, DMAP, CHCl₃, 39%

### Scheme I-3

### (+/−)-2-Butylheptadecanoic acid (3-2)

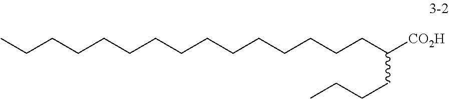 [SMILES: CCCCCCCCCCCCCCCC(CCCC)C(=O)O]

A solution of i-Pr₂NH (16.40 g, 0.162 mol) in anhydrous THF (200 mL) was cooled to −20° C. under nitrogen, the n-BuLi (2.5 M in hexanes, 65 mL, 0.163 mol) was added over a period of 30 minutes. The mixture was allowed to stir for 30 minutes at −20° C., then a solution of heptadecanoic acid (3-1, 20.0 g, 74 mmol) in THF (50 mL) was added over 20 minutes. The solution was stirred for 20 minutes (−20° C.) and a solution of n-butylbromide (10.1 g, 74 mmol) in THF (20 mL) was added dropwise over a period of 10 minutes. The mixture was allowed to stir for 30 minutes after the addition was complete, then was warmed to room temperature and was stirred for 16 hours. The reaction mixture was poured into cold (0° C.) 10% aq. HCl (250 mL) and the solution was stirred for 20 minutes. The solution was warmed to room temperature and was extracted with petroleum ether (2×200 mL) and the combined organic phases were washed with brine (250 mL), dried (MgSO₄), filtered, and concentrated in vacuo to give crude 3-2 as a sticky, pale yellow semi-solid. Crude 3-2 was dissolved in CH₂Cl₂ (100 mL) and silica gel (60 g, type: ZCX-100-200 mesh) was added. The solvent was removed in vacuo and the silica gel containing adsorbed 3-2 was loaded atop a silica gel column (300 g, type: ZCX-100-200 mesh) which was eluted with a gradient of petroleum ether:EtOAc from 100:0 to 90:10. Qualified fractions were pooled and concentrated in vacuo to afford 3-2 (9.00 g, 27.6 mmol, 37%) as a pale yellow solid. ¹H-NMR (400 MHz, CDCl₃): δ 2.38 (m, 1H), 1.64 (m, 2H), 1.51 (m, 2H), 1.20-1.40 (30H), 0.85-0.92 (6H).

### (+/−)-2-Butylheptadecanoyl chloride (3-3)

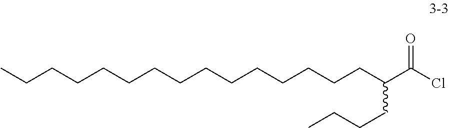 [SMILES: CCCCCCCCCCCCCCCC(CCCC)C(=O)Cl]

To a solution of 3-2 (9.00 g, 27.6 mmol) in CH₂Cl₂ (45 mL), under nitrogen at room temperature, was added thionyl chloride (45 mL, 73.8 g, 0.62 mol) over a period of 15 minutes. The mixture was stirred at room temperature for 30 minutes, then was warmed to 40° C. and was stirred for 6 hours. The mixture was cooled to room temperature and then was concentrated in vacuo, the residue was diluted with toluene (50 mL) and the solvent was removed in vacuo, this operation was repeated one more time to give crude 3-3 (8.5 g, assumed 24.6 mmol, assumed 89%). ¹H-NMR (400 MHz, CDCl₃): δ 2.77 (m, 1H), 1.75 (m, 2H), 1.56 (m, 2H), 1.20-1.41 (30H), 0.83-0.94 (6H).

### (2R)-3-((+/−2-Butylheptadecanoyl)oxy)-2-hydroxypropyl (2-(trimethylammonio)ethyl) phosphate (3-4)

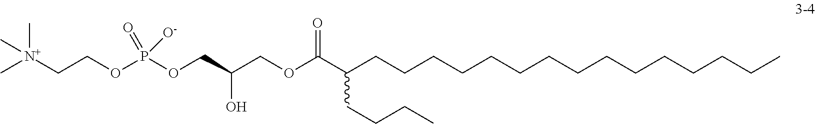 [SMILES: CCCCCCCCCCCCCCCC(CCCC)C(=O)OC[C@@H](O)COP(=O)([O-])OCC[N+](C)(C)C]

A mixture of (R)-2,3-dihydroxypropyl (2-(trimethylammonio)ethyl) phosphate (sn-glycero-3-phosphocholine (6.20 g, 24.1 mmol) and dibutyltin oxide (6.33 g, 25.43 mmol) was dissolved in isopropanol (400 mL), under nitrogen. After solution was achieved the mixture was heated under reflux for 4 hours, then was cooled to room temperature. To this cooled solution was added Et₃N (2.93 g, 29 mmol), in one portion, followed by the addition of 3-3 (10.0 g, assumed 29.0 mmol). The mixture was allowed to stir for 16 hours at room temperature then was cast into water (100 mL) and the resulting solid was removed by filtration. The solid was dissolved in CH₂Cl₂ (250 mL) and silica gel (60 g, type: ZCX-100-200 mesh) was added. The solvent was removed in vacuo and the silica gel containing adsorbed 3-4 was loaded atop a silica gel column (300 g, type: ZCX-100-200 mesh) which was eluted with a gradient of CH₂Cl₂:MeOH from 100:0 to 90:10. Qualified fractions were pooled and concentrated in vacuo to afford 3-4 (9.50 g, 16.8 mmol, 70%) as a white solid. LC-MS (+ mode): RT 2.295 min, Calcd. For C₂₉H₆₀NO₇P: 565.41; Found: 566.52 (M+H⁺); ¹H-NMR (300 MHz, CD₃OD): d 3.80-4.35 (7H), 3.58-3.70 (3H), 3.22 (s, 9H), 2.38 (m, 1H), 1.40-1.70 (4H), 1.19-1.40 (30H), 0.85-0.92 (6H).

### (2R)-3-((+/−2-butylheptadecanoyl)oxy)-2-(icosanoyloxy)propyl (2-(trimethylammonio) ethyl) phosphate (3)

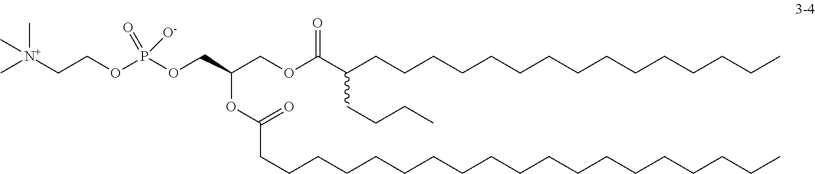 [SMILES: CCCCCCCCCCCCCCCCCCCC(=O)O[C@H](COC(=O)C(CCCC)CCCCCCCCCCCCCCC)COP(=O)([O-])OCC[N+](C)(C)C]

To a room temperature solution of 3-4 (5.00 g, 8.83 mmol) and icosanoic acid (3.31 g, 10.59 mmol) in CHCl₃ (100 mL), under nitrogen, was added in order DMAP (0.54 g, 4.42 mmol) and DCC (5.47 g, 26.51 mmol). The reaction mixture was stirred for 72 hours at room temperature, solid materials were removed by filtration through a sintered glass funnel and the filtrate was concentrated in vacuo to give crude 3. Crude 3 was dissolved in CH₂Cl₂ (100 mL) and silica gel (25 g, type: ZCX-100-200 mesh) was added. The solvent was removed in vacuo and the silica gel containing adsorbed 3 was loaded atop a silica gel column (150 g, type: ZCX-100-200 mesh) which was eluted with a gradient of CH₂Cl₂:MeOH from 100:0 to 90:10, then the column was eluted with CHCl₃:MeOH:H₂O (78:20:2). Qualified fractions were pooled and concentrated in vacuo to afford 3 (3.00 g, 3.49 mmol, 39%) as a white solid. LC-MS (+ mode): RT 3.422 min, Calcd. For C₄₉H₉₈NO₈P 859.70. Found 861.10; ¹H-NMR (400 MHz, CDCl₃): δ 5.24 (m, 1H), 4.40-4.55 (3H), 4.20 (m, 1H), 3.85-4.08 (4H), 3.42 (s, 9H), 2.25-2.40 (3H), 1.40-1.65 (6H), 1.15-1.40 (62H), 0.86-0.93 (9H); ³¹P-NMR (400 MHz, CDCl₃): 45.29 ppm.

### Example 4 Synthesis of Compound I-4

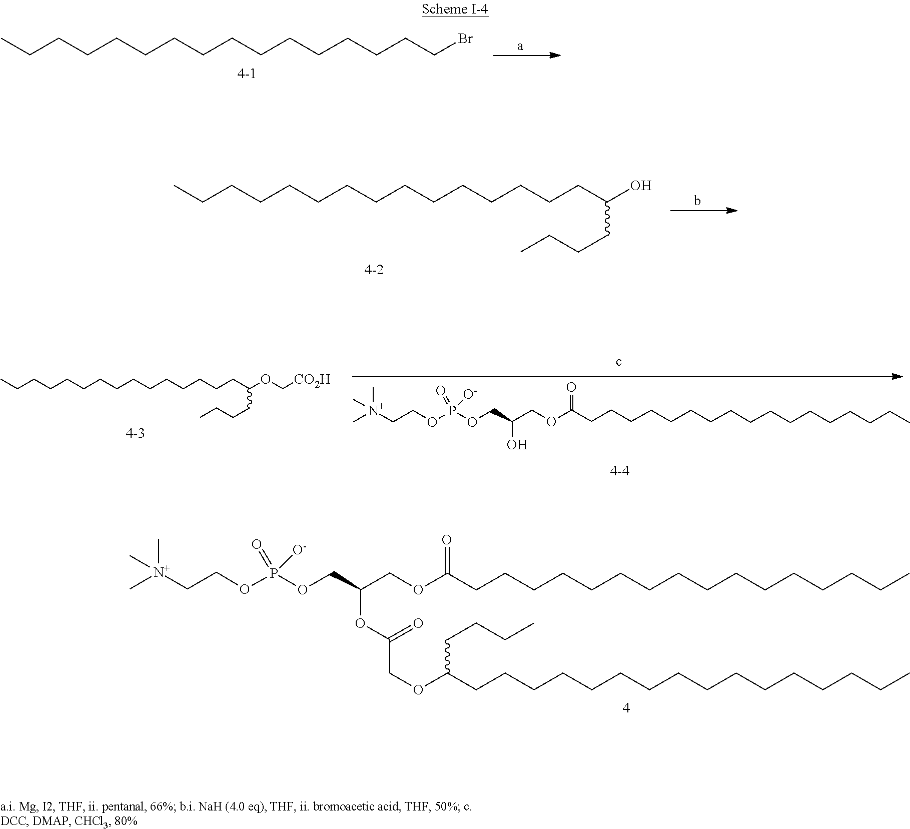

### (+/−)-icosan-5-ol (4-2)

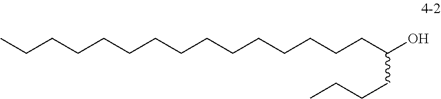 [SMILES: CCCCCCCCCCCCCCCC(O)CCCC]

Dried magnesium turnings (7.40 g, 0.304 mol) were covered with dry THF (125 mL), under nitrogen, and solid iodine (0.295 g, 1.16 mmol) was added. The mixture was warmed to 40° C. and after the color of iodine had disappeared a solution of 1-bromopentadecane (4-1, 76.0 g, 0.257 mol) in THF (150 mL) was added at such a rate that the temperature of the reaction mixture did not exceed 55° C. After the addition was complete the temperature of the mixture was allowed to cool to 40° C. and was maintained at that temperature for 30 minutes. The Grignard solution was then cooled to 20° C. and a solution of pentanal (20.0 g, 0.232 mol) in THF (50 mL) was added at such a rate that the temperature of the reaction mixture did not exceed 40° C. The mixture was stirred at 40° C. for 18 hours after the addition was complete, then was cooled to 10° C. and the reaction was quenched by the addition of 25% aq. NH₄Cl solution (500 mL). The mixture was filtered through a sintered glass funnel and then was extracted with hexanes (2×500 mL). The combined organic phases were dried (Na₂SO₄), filtered, and concentrated in vacuo to give crude 4-2 as a pale yellow, oil. Crude 4-2 was purified by chromatography on a column of silica gel (750 g, 60-200 mesh) packed with hexanes-Et₂O (99:1) and eluted with a gradient of hexanes-Et₂O (99:1 to 85:15). Qualified fractions were combined and concentrated in vacuo to afford 4-2 (46.0 g, 0.151 mol, 65%) as a clear, colorless oil. TOF-MS Calcd. for C₂₀H₄₂O 298.32, Found 316.30 (M+NH₄⁺); ¹H-NMR (400 MHz, CDCl₃): δ 3.59 (m, 1H), 1.23-1.50 (34H), 0.84-0.95 (6H).

### 2-([+/−]-icosan-5-yloxy)acetic acid (4-3)

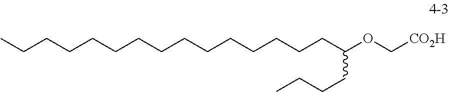 [SMILES: CCCCCCCCCCCCCCCC(CCCC)OCC(=O)O]

To NaH (90%, 1.79 g, 67.1 mmol), in dry THF (25 mL) under nitrogen at room temperature, was added, dropwise, a solution of 4-2 (5.00 g, 16.74 mmol) in THF (25 mL) over a period of 30 minutes. The mixture was allowed to stir for 2 hours at room temperature, then a solution of bromoacetic acid (2.56 g, 18.42 mmol) in THF (15 mL) was added dropwise over 15 minutes. The mixture was allowed to stir for 30 minutes after the addition was complete and n-Bu₄NI (0.31 g, 1.00 mmol) was added in 1 portion, followed by dry DMF (10 mL) and the mixture was warmed to 40° C. and was stirred for 48 hours. The mixture was cooled to room temperature and MeOH (10 mL) was slowly added, and the mixture was cast into water (100 mL). The solution was extracted with hexanes (2×100 mL) and the combined organic phases were washed with water (3×75 L) and dried (Na₂SO₄). Filtration and concentration in vacuo gave crude 4-3 which was further azeotropically dried with toluene (3×40 mL) and purified by chromatography on a column of silica gel (130 g, 60-200 mesh), packed with hexanes-EtOAc (95:5) and eluted with a gradient of hexanes-EtOAc (95:5 to 80:20). Qualified fractions were combined and concentrated in vacuo to give 4-3 (3.00 g, 8.41 mmol, 50%) as a clear, viscous oil. TOF-MS: Calcd. for C₂₂H₄₄O₃ 356.33, Found 355.5 (M−H⁺); ¹H-NMR (400 MHz, CDCl₃): δ 4.07 (s, 2H), 3.44 (m, 1H), 1.44-1.58 (4H), 1.25-1.40 (30H), 0.84-0.95 (6H).

### (2R)-2-(2-([+/−]-icosan-5-yloxy)acetoxy)-3-(stearoyloxy)propyl (2-(trimethylammonio)ethyl) phosphate (4)

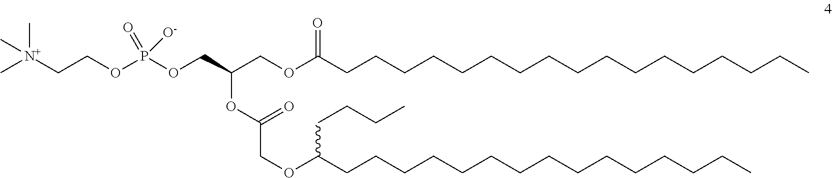 [SMILES: CCCCCCCCCCCCCCCCCC(=O)OC[C@H](COP(=O)([O-])OCC[N+](C)(C)C)OC(=O)COC(CCCC)CCCCCCCCCCCCCCC]

(R)-2-hydroxy-3-(stearoyloxy)propyl (2-(trimethylammonio)ethyl) phosphate (4-4, Avanti Polar Lipids #855775, 2.20 g, 4.20 mmol) and 4-3 (1.80 g, 5.05 mmol) were dissolved in CHCl₃ (50 mL) at room temperature under nitrogen. To this solution was added in order DMAP (0.26 g, 2.13 mmol) and DCC (2.60 g, 12.60 mmol) and the resulting reaction mixture was stirred at room temperature for 72 hours. Suspended solids were removed by filtration and the filtrate was concentrated in vacuo to afford crude 4 as a sticky semi-solid that was dissolved in a mixture of CHCl₃/MeOH/ammonium hydroxide (3 mL, 80:20:2) and was purified by medium pressure chromatography over a column of silica gel (60 g, 60-200 mesh) using a gradient from CHCl₃/MeOH/ammonium hydroxide (80:20:2) to CHCl₃/MeOH/water (80:20:2). Qualified fractions were pooled, and non-qualified fractions containing 4 were concentrated in vacuo and subjected to further chromatography under identical conditions. Qualified fractions from both chromatographic runs were combined and concentrated in vacuo and dried azeotropically by dissolution in i-PrOH/CHCl₃ (1:1, 10 mL) and concentration in vacuo. The azeotropic drying was repeated 3 additional times. The product was then dissolved in cyclohexane/EtOH (96:4), frozen and lyophilized to provide 4 (2.88 g, 3.34 mmol, 80%) as a white solid. TOF-MS: Calcd. for C₄₈H₉₆NO₉P 861.68, Found 863.0 (M+H⁺); ¹H-NMR (400 MHz, CDCl₃): δ 5.27 (m, 1H), 4.30-4.47 (3H), 4.09-4.26 (3H), 3.83 (m, 2H), 3.39 (s, 9H), 3.35 (m, 1H), 2.32 (m, 2H), 1.45-1.70 (8H), 1.23-1.43 (58H), 0.85-0.96 (9H).

### Example 5 Synthesis of Compound I-5

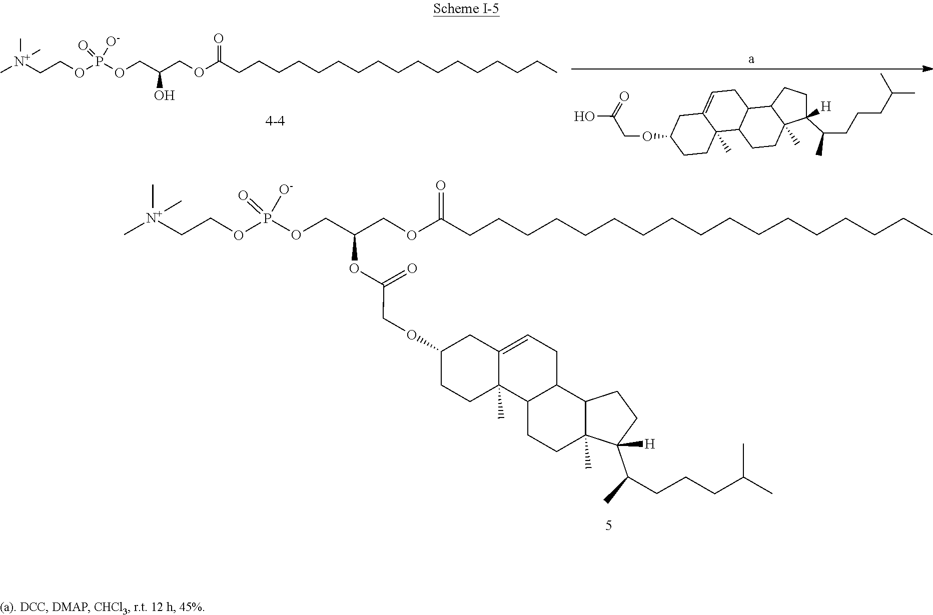 [SMILES: CCCCCCCCCCCCCCCCCC(=O)OC[C@@H](O)COP(=O)([O-])OCC[N+](C)(C)C ; CC(C)CCC[C@@H](C)[C@H]1CCC2C3CC=C4C[C@@H](OCC(=O)O)CC[C@]4(C)C3CC[C@@]21C ; CCCCCCCCCCCCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1]

### (2R)-2-(2-(((3S,10R,13R,17R)-10, 13-dim ethyl-17-((R)-6-m ethylheptan-2-yl)-2,3,4,7,8,9,10,11,12,13,14,15,16,17-tetradecahydro-1H-cyclopenta[a]phenanthren-3-yl)oxy)acetoxy)-3-(stearoyloxy)propyl (2-(trimethylammonio)ethyl) phosphate (5)

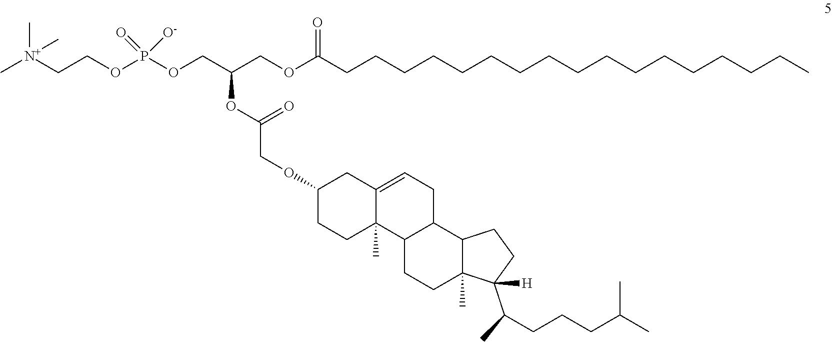 [SMILES: CCCCCCCCCCCCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1]

To a solution of 4-4 (2.0 g, 3.8 mmol) and 2-(((3S,10R,13R,17R)-10,13-dimethyl-17-((R)-6-methylheptan-2-yl)-2,3,4,7,8,9,10,11,12,13,14, 15,16,17-tetradecahydro-1H-cyclopenta[a]phenanthren-3-yl)oxy)acetic acid (2.04 g, 4.59 mmol) in DCM (50 mL), cooled in an ice-water bath under nitrogen, was added in order DCC (2.3 g, 11.4 mmol) and DMAP (0.234 g, 1.9 mmol). The mixture was stirred for 30 minutes at 0° C. after the additions were complete, then was warmed to room temperature and was allowed to stir for 48 hours. The mixture was concentrated in vacuo and the residue was dissolved in CH₂Cl₂ (100 mL) and 30 g silica gel was added. The solvent was removed in vacuo and the silica gel containing adsorbed 5 was loaded atop a silica gel column (250 g, type: ZCX-100-200 mesh) which was eluted with a gradient of CH₂Cl₂:MeOH from 100:0 to 80:20 followed by DCM:MeOH:H₂O (v/v/v=65:25:4). Eluent was concentrated and freeze dried to afford 5 (1.72 g, HPLC=95.6%) as an off white solid. Yield=45.41%. LC-MS (+ mode): RT 2.47 min, Calcd. for C₅₅H₁₀₀NO₉P 949.71, Found 950.90 (M+H⁺); ¹H NMR (400 MHz, Chloroform-d) δ 4.31 (s, 3H), 4.22-4.07 (m, 3H), 3.98 (t, J=6.5 Hz, 2H), 3.80 (s, 2H), 3.36 (s, 10H), 2.25 (dt, J=20.0, 10.3 Hz, 4H), 2.06-1.81 (m, 5H), 1.56 (dd, J=9.6, 4.7 Hz, 3H), 1.47 (ddt, J=20.7, 10.2, 5.5 Hz, 3H), 1.37-1.25 (m, 10H), 1.25 (s, 21H), 1.18-1.09 (m, 3H), 1.12-0.96 (m, 10H), 0.99 (s, 4H), 0.96-0.83 (m, 13H), 0.68 (s, 3H).

### Example 6 Synthesis of Compound I-6

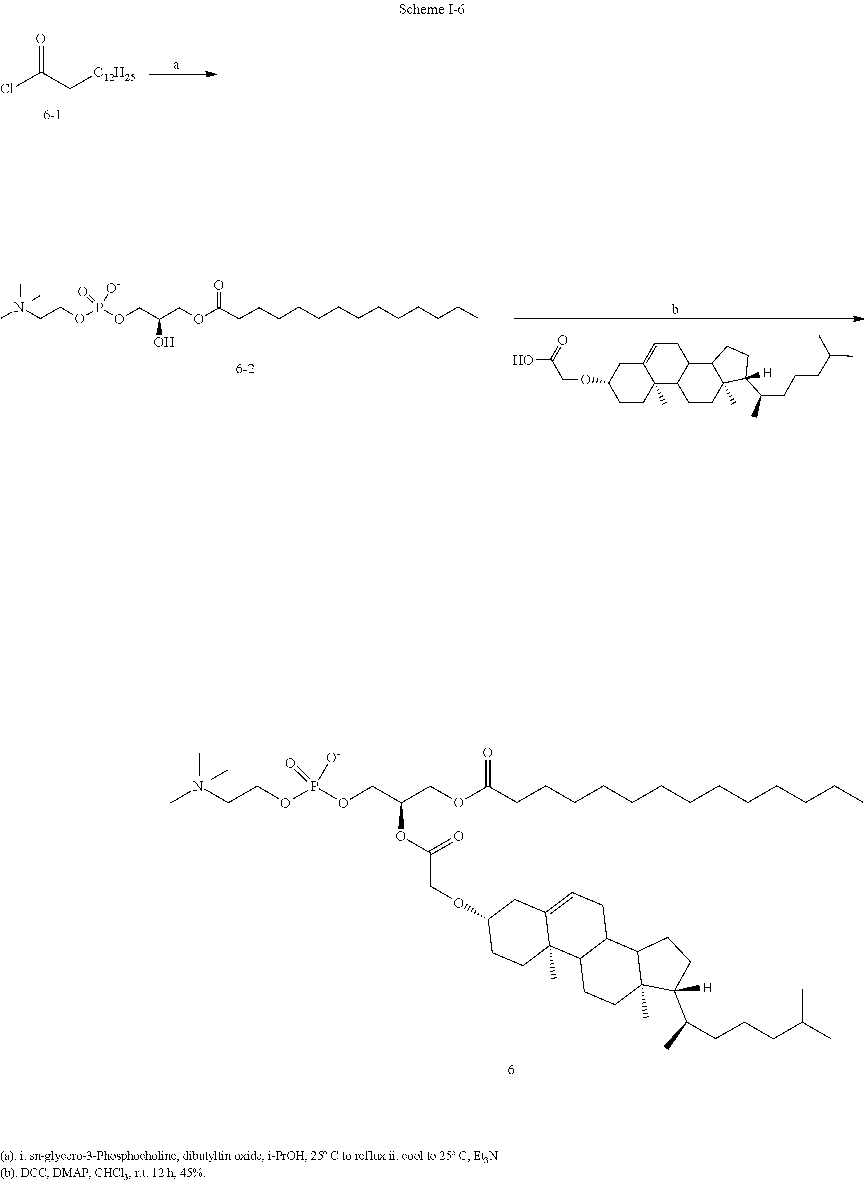

### (R)-2-hydroxy-3-(tetradecanoyloxy)propyl (2-(trimethylammonio)ethyl) phosphate (6-2)

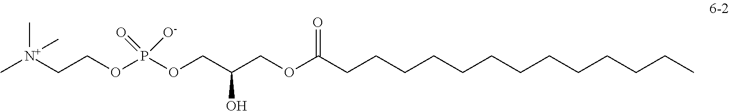 [SMILES: CCCCCCCCCCCCCC(=O)OC[C@@H](O)COP(=O)([O-])OCC[N+](C)(C)C]

A mixture of sn-glycero-3-Phosphocholine (3.12 g, 1.0 eq) and DBTO (3.03 g, 1.0 eq) was dissolved in 2-propanol (120 ml, 40 V) in a 300 mL round-bottom flask at 25° C. with N₂ protection. The reaction mixture was warmed to 82° C. reflux and stirred for 4 h. The mixture was cooled to 25° C. and TEA (3 g, 2 eq) was added followed by myristoyl chloride (3 g, 1 eq) into the reactor. The reaction mixture was stirred 15 h at 25° C. Water was (10 V) charged into the reaction and filtered. The crude mixture was dissolved in CH₂Cl₂ (250 mL) and silica gel (50 g, type: ZCX-100-200 mesh) was added. The solvent was removed in vacuo and the silica gel containing adsorbed 6-2 was loaded atop a silica gel column (300 g, type: ZCX-100-200 mesh) which was eluted with a gradient of CH₂Cl₂:MeOH from 100:0 to 90:10 followed by DCM:MeOH:H₂O ((v/v/v) (65:25:4)) to elute the product. Qualified fractions were pooled and concentrated in vacuo to afford 6-2 (3.55 g, 62%) as a white solid. LC-MS (+ mode): RT 0.783 min, Calcd. for Chemical Formula: C₂₂H₄₆NO₇P, 467.50; Found 468.50; 1H-NMR (400 MHz, CD₃OD): d 4.31 (m, 2H), 4.20 (m, 2H), 4.14 (3H), 4.07 (m, 2H), 3.31 (s, 9H), 2.34 (m, 2H), 1.61 (m, 2H), 1.22-1.32 (21H), 0.90 (t, J=6.0 Hz, 3H).

### (2R)-2-(2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetoxy)-3-(tetradecanoyloxy)propyl (2-(trimethylammonio)ethyl) phosphate (6)

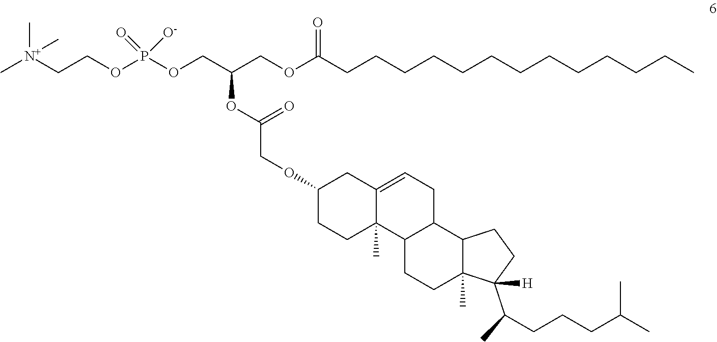 [SMILES: CCCCCCCCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1]

To a solution of 6-2 (2.0 g) and 2-(((3S,10R,13R,17R)-10,13-dimethyl-17-((R)-6-methylheptan-2-yl)-2,3,4,7,8,9,10,11,12,13,14, 15,16,17-tetradecahydro-1H-cyclopenta[a]phenanthren-3-yl)oxy)acetic acid (2.28 g) in DCM (60 mL), cooled in an ice-water bath under nitrogen, was added in order DCC (2.65 g) and DMAP (0.234 g). The mixture was stirred for 30 minutes at 0° C. after the additions were complete, then was warmed to room temperature and was allowed to stir for 48 hours. The mixture was concentrated in vacuo and the residue was dissolved in CH₂Cl₂ (100 mL) and 30 g silica gel was added. The solvent was removed in vacuo and the silica gel containing adsorbed 6 was loaded atop a silica gel column (250 g, type: ZCX-100-200 mesh) which was eluted with a gradient of CH₂Cl₂:MeOH from 100:0 to 80:20 followed by DCM:MeOH:H₂O (v/v/v=65:25:4). Eluent was concentrated and freeze dried to afford 6 (1.72 g, HPLC=95.6%) as off white solid. Yield=43.6%. LC-MS (+ mode): RT 2.46 min, Calcd. for C₅₁H₉₂NO₉P 894.27, Found 894.8 (M⁺); ¹H NMR: (400 MHz, Chloroform-d) δ 5.37-5.24 (m, 2H), 4.31 (s, 3H), 4.22-4.07 (m, 3H), 3.98 (t, J=6.4 Hz, 2H), 3.86 (s, 2H), 3.80-3.35 (s, 10H), 2.26 (q, J=7.8 Hz, 4H), 2.06-1.89 (m, 2H), 1.89-1.78 (m, 3H), 1.51 (tdt, J=20.0, 9.9, 5.6 Hz, 5H), 1.26 (d, J=5.7 Hz, 24H), 1.14 (dd, J=10.4, 5.8 Hz, 3H), 1.13-0.99 (m, 3H), 1.02-0.84 (m, 21H), 0.86 (d, J=1.8 Hz, 3H), 0.68 (s, 3H).

### Example 7 Synthesis of Compound I-7

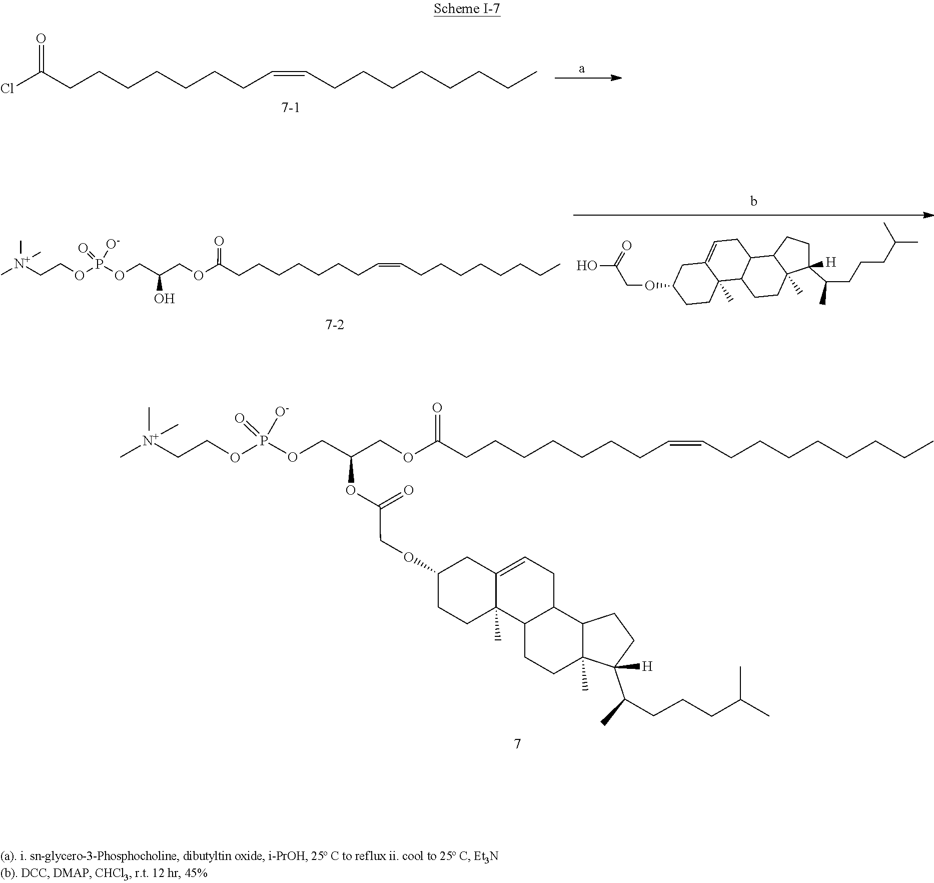

### (R)-2-hydroxy-3-(oleoyloxy)propyl (2-(trimethylammonio)ethyl) phosphate (7-2)

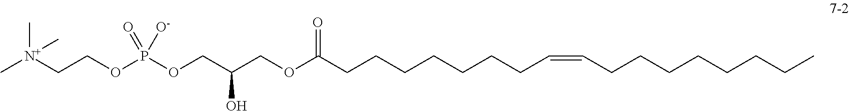 [SMILES: CCCCCCCC/C=C\CCCCCCCC(=O)OC[C@@H](O)COP(=O)([O-])OCC[N+](C)(C)C]

A mixture of sn-glycero-3-Phosphocholine (2.57 g, 1.0 eq) and DBTO (2.49 g, 1.0 eq) was dissolved in 2-propanol (100 ml, 40 V) in a 300 mL round-bottom flask at 25° C. with N₂ protection. The reaction mixture was warmed to 82° C. reflux and stirred for 4 h. The mixture was cooled to 25° C. and TEA (3 g, 2 eq) was added followed by oleoyl chloride (3 g, 1 eq) into the reactor. The reaction mixture was stirred 15 h at 25° C. Water was (10 V) charged into the reaction and filtered. The crude mixture was dissolved in CH₂Cl₂ (250 mL) and silica gel (50 g, type: ZCX-100-200 mesh) was added. The solvent was removed in vacuo and the material was loaded onto a silica gel column (300 g, type: ZCX-100-200 mesh) and eluted with a gradient of CH₂Cl₂:MeOH from 100:0 to 90:10 followed by DCM:MeOH:H₂O ((v/v/v) (65:25:4)) to elute the product. Qualified fractions were pooled and concentrated in vacuo to afford 7-2 (3.56 g, 68%) as a white solid.

LC-MS (+ mode): RT 0.880 min, Calcd. for Chemical Formula: C₂₆H₅₂NO₇P, 521.68; Found 522.60; 1H-NMR (400 MHz, CD₃OD): 5.34 (dt, 2H), 4.32 (m, 2H), 4.30 (m, 2H), 4.14 (2H), 3.98 (m, 2H), 3.22 (s, 9H), 2.35 (m, 2H), 2.05 (m, 4H) 1.62 (m, 2H), 1.22-1.32 (m, 22H), 0.90 (t, J=6.0 Hz, 3H).

### (2R)-2-(2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetoxy)-3-(oleoyloxy)propyl (2-(trimethylammonio)ethyl) phosphate (7)

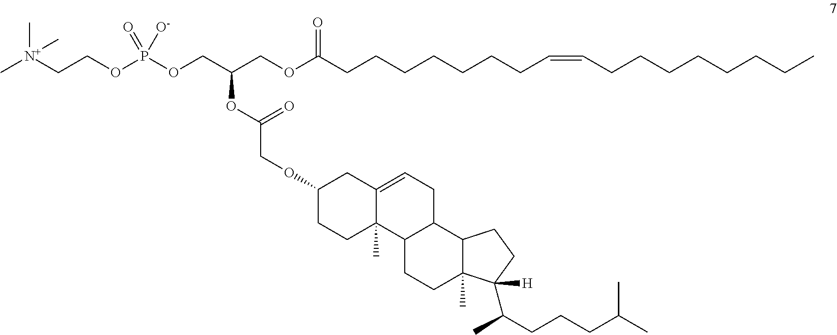 [SMILES: CCCCCCCC/C=C\CCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1]

To a solution of 7-2 (2.0 g) and 2-(((3S,10R,13R,17R)-10,13-dimethyl-17-((R)-6-methylheptan-2-yl)-2,3,4,7,8,9,10,11,12,13,14, 15,16,17-tetradecahydro-1H-cyclopenta[a]phenanthren-3-yl)oxy)acetic acid (2.28 g) in DCM (60 mL), cooled in an ice-water bath under nitrogen, was added in order DCC (2.65 g) and DMAP (0.261 g). The mixture was stirred for 30 minutes at 0° C. after the additions were complete, then was warmed to room temperature and was allowed to stir for 48 hours. The mixture was concentrated in vacuo and the residue was dissolved in CH₂Cl₂ (100 mL) and 30 g silica gel was added. The solvent was removed in vacuo, loaded atop a silica gel column (250 g, type: ZCX-100-200 mesh) and was eluted with a gradient of CH₂Cl₂:MeOH from 100:0 to 80:20 followed by DCM:MeOH:H₂O (v/v/v=65:25:4). Eluent was concentrated and freeze dried to afford 7 (1.65 g, HPLC=95.6%) as off white solid. Yield=44.6%. LC-MS (+ mode): RT 2.23 min, Calcd. for C₅₅H₉₈NO₉P 948.36, Found 948.90 (M⁺); ¹H NMR: (300 MHz, Chloroform-d) δ 5.39-5.25 (m, 4H), 4.30 (s, 3H), 4.14 (q, J=7.1, 6.2 Hz, 3H), 3.98 (t, J=6.6 Hz, 2H), 3.80 (s, 2H), 3.64 (s, 1H), 3.36 (s, 9H), 2.42-2.22 (m, 4H), 2.01 (q, J=6.1, 5.1 Hz, 7H), 1.91-1.80 (m, 10H), 1.62-1.43 (m, 3H), 1.29 (t, J=8.8 Hz, 26H), 1.19-0.95 (m, 6H), 0.99 (s, 3H), 0.89 (dd, J=14.6, 6.7 Hz, 13H), 0.67 (s, 3H).

### Example 8 Synthesis of Compound I-8

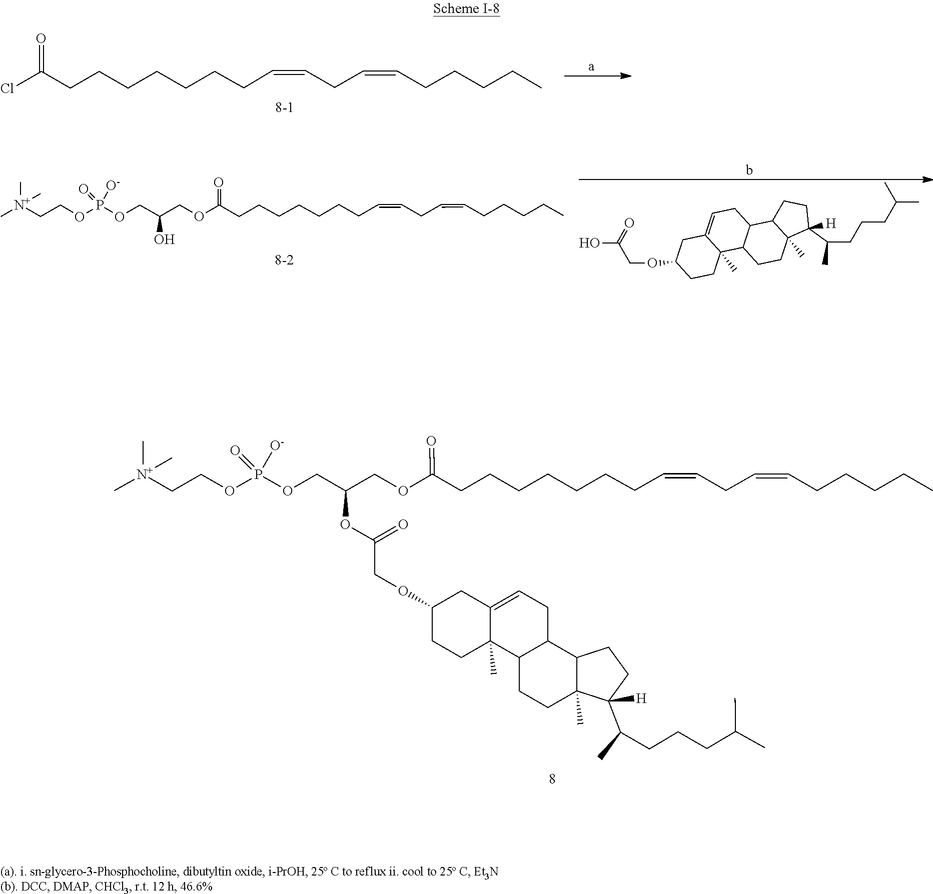

### (R)-2-hydroxy-3-(((9Z,12Z)-octadeca-9,12-dienoyl)oxy)propyl (2-(trimethylammonio)ethyl) phosphate (8-2)

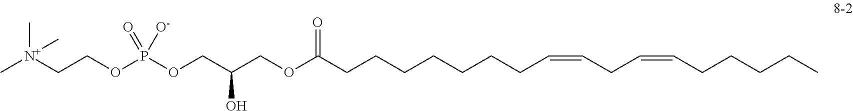 [SMILES: CCCCC/C=C\C/C=C\CCCCCCCC(=O)OC[C@@H](O)COP(=O)([O-])OCC[N+](C)(C)C]

A mixture of sn-glycero-3-phosphocholine (2.59 g, 1.0 eq) and DBTO (2.51 g, 1.0 eq) was dissolved in 2-propanol (104 ml, 40 V) in a 300 mL round-bottom flask at 25° C. with N₂ protection. The reaction mixture was warmed to 82° C. reflux and stirred for 4 h. The mixture was cooled to 25° C. and TEA (3 g, 2 eq) was added followed by linoleoyl chloride (3 g, 1 eq) into the reactor. The reaction mixture was stirred 15 h at 25° C. Water was (10 V) charged into the reaction and filtered. The crude mixture was dissolved in CH₂Cl₂ (250 mL) and silica gel (30 g, type: ZCX-100-200 mesh) was added. The solvent was removed in vacuo and the silica gel containing adsorbed 8-2 was loaded atop a silica gel column (300 g, type: ZCX-100-200 mesh) which was eluted with a gradient of CH₂Cl₂:MeOH from 100:0 to 90:10 followed by DCM:MeOH:H₂O ((v/v/v) (65:25:4)) to elute the product. Qualified fractions were pooled and concentrated in vacuo to afford 8-2 (3.6 g, 69%) as a white solid. LC-MS (+ mode): RT 0.82 min, Calcd. for Chemical Formula: C₂₆H₅₀NO₇P, 519.66; Found 520.60; 1H-NMR (400 MHz, CD₃OD): 5.34 (m, 4H), 4.30 (m, 2H), 4.28 (m, 2H), 4.15 (3H), 3.95 (m, 2H), 3.29 (s, 9H), 2.77 (m, 2H), 2.35 (m, 2H), 2.07 (m, 4H), 1.62 (m, 2H), 1.22-1.32 (m, 15H), 0.90 (t, J=6.0 Hz, 3H).

### (2R)-2-(2-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)acetoxy)-3-(((9Z,12Z)-octadeca-9,12-dienoyl)oxy)propyl (2-(trimethylammonio)ethyl) phosphate (8)

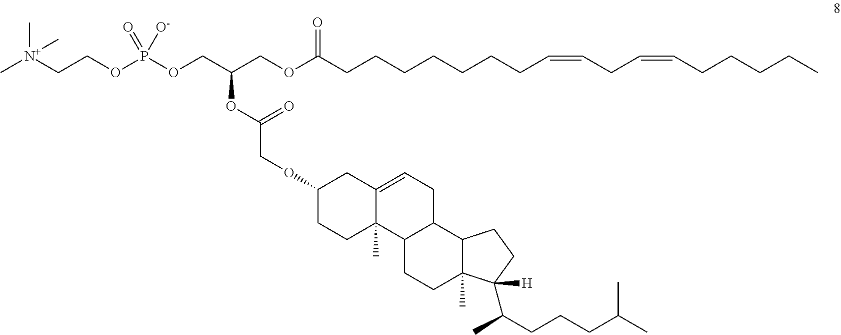 [SMILES: CCCCC/C=C\C/C=C\CCCCCCCC(=O)OC[C@H](COP(=O)([O-])OCC[N+](C)(C)C)OC(=O)CO[C@H]1CC[C@@]2(C)C(=CCC3C2CC[C@@]2(C)C3CC[C@@H]2[C@H](C)CCCC(C)C)C1]

To a solution of 8-2 (2.0 g) and 2-(((3S,10R,13R,17R)-10,13-dimethyl-17-((R)-6-methylheptan-2-yl)-2,3,4,7,8,9,10,11,12,13,14, 15,16,17-tetradecahydro-1H-cyclopenta[a]phenanthren-3-yl)oxy)acetic acid (2.05 g) in DCM (60 mL), cooled in an ice-water bath under nitrogen, was added in order DCC (2.4 g) and DMAP (0.235 g). The mixture was stirred for 30 minutes at 0° C. after the additions were complete, then was warmed to room temperature and was allowed to stir for 48 hours. The mixture was concentrated in vacuo and the residue was dissolved in CH₂Cl₂ (100 mL) and 30 g silica gel was added. The solvent was removed in vacuo and the silica gel was loaded atop a silica gel column (250 g, type: ZCX-100-200 mesh) which was eluted with a gradient of CH₂Cl₂:MeOH from 100:0 to 80:20 followed by DCM:MeOH:H₂O (v/v/v=65:25:4). Eluent was concentrated and freeze dried to afford 8 (1.75 g, HPLC=97%) as off white solid. Yield=46.5%. LC-MS (+ mode): RT 2.37 min, Calcd. for C₅₅H₉₆NO₉P 946.34, Found 946.85 (M⁺); ¹H NMR: (400 MHz, Chloroform-d) δ 5.44-5.25 (m, 6H), 4.33 (s, 3H), 4.14 (q, J=7.1, 6.6 Hz, 3H), 4.03-3.93 (m, 2H), 3.86-3.79 (m, 2H), 3.37 (s, 10H), 3.05 (s, 2H), 2.77 (t, J=6.5 Hz, 2H), 2.32-2.18 (m, 3H), 2.10-1.97 (m, 5H), 1.97-1.78 (m, 3H), 1.57 (s, 4H), 1.58-1.41 (m, 4H), 1.41-1.22 (m, 19H), 1.21-0.83 (m, 25H), 0.67 (s, 3H).

### Example 9 Synthesis of Compound I-9

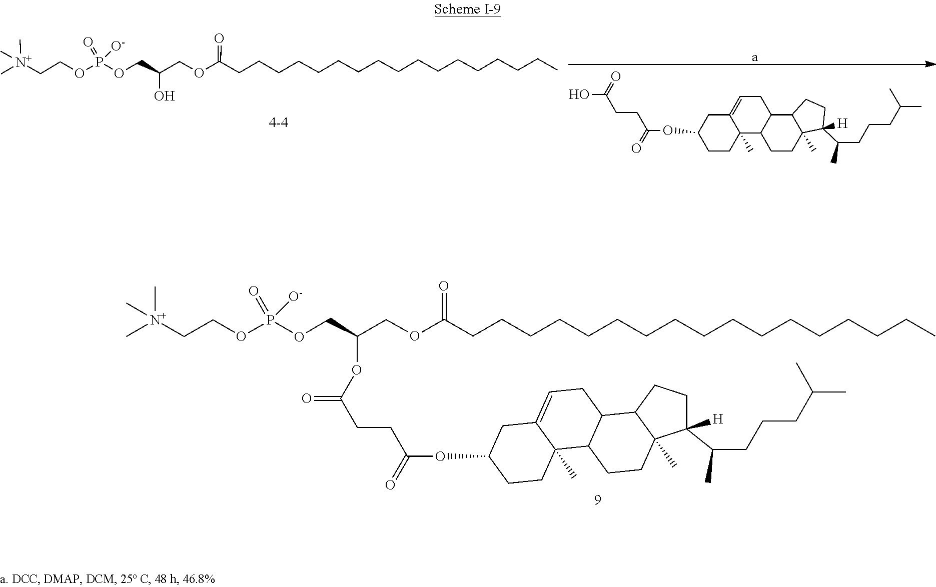

### (2R)-2-((4-(((3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoyl)oxy)-3-(stearoyloxy)propyl (2-(trimethylammonio)ethyl) phosphate (9)

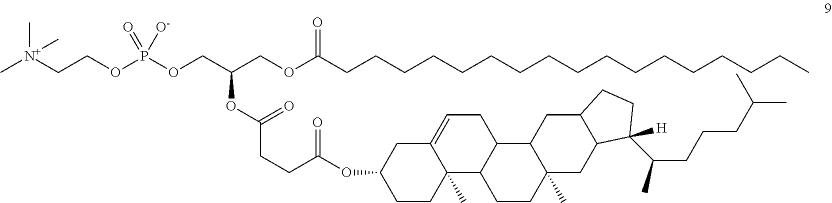 [SMILES: CCCCCCCCCCCCCCCCCC(=O)OC[C@H](COP(=O)([O-])OCC[N+](C)(C)C)OC(=O)CCC(=O)O[C@H]1CC[C@@]2(C)C(=CCC3C4CC5CC[C@H]([C@H](C)CCCC(C)C)C5C[C@@]4(C)CCC32)C1]

To 45 mL anhydrous DCM kept in 250 mL round-bottom flask kept under argon was charged with (R)-2-hydroxy-3-(stearoyloxy)propyl(2-(trimethylammonio)ethyl) phosphate (4-4, 1.5 g, 1.0 eq) and cholesteryl hemisuccinate (1.67 g, 1.2 eq) in dry DCM (45 mL, 30 V). The reaction mixture was cooled to 0° C. and charged with DCC (1.79 g, 3 eq) and DMAP (175.5 mg, 0.5 eq) and stirred for 48 h at 20-25° C. The reaction was mixed with 50 g silica gel and solvent was removed under reduced pressure at 35° C. to adsorb the material. The dry silica gel powder was applied onto atmospheric silica gel column (300 g, type: ZCX-2,100-200 mesh) eluted with DCM:MeOH, gradient from 1/0 to 5:1, then eluted with DCM:MeOH:H₂O (v/v/v=65:25:4) and collected product. The eluent was concentrated and freeze dried to afford 9 (1.35 g, HPLC=98.2%) as light-yellow solid. Yield=46.80%. LC-MS (+ mode): RT 2.39 min, Calcd. for C57H102NO10P 992.41, Found 992.90 (M⁺); ¹H NMR: (400 MHz, Chloroform-d) δ 5.39-5.33 (m, 1H), 5.21 (qd, J=5.4, 3.1 Hz, 1H), 4.58 (tt, J=12.2, 5.6 Hz, 1H), 4.36 (dd, J=12.2, 3.2 Hz, 1H), 4.32 (d, J=6.8 Hz, 2H), 4.15 (dd, J=12.1, 7.0 Hz, 1H), 3.97 (t, J=6.3 Hz, 2H), 3.82 (d, J=4.8 Hz, 2H), 3.49 (s, 2H), 3.37 (s, 9H), 2.61 (dtt, J=12.7, 10.1, 6.4 Hz, 4H), 2.29 (dt, J=11.9, 6.1 Hz, 4H), 1.99 (ddt, J=21.4, 17.0, 4.2 Hz, 2H), 1.85 (ddd, J=13.3, 8.8, 4.5 Hz, 3H), 1.65-1.40 (m, 4H), 1.37-1.25 (m, 37H), 1.12 (qd, J=11.0, 9.5, 5.0 Hz, 5H), 1.02 (s, 3H), 0.99 (d, J=8.5 Hz, 1H), 0.97-0.83 (m, 15H), 0.68 (s, 3H).

### Example 10 General Synthesis of Compounds I-2-1 to I-2-9, I-5-1 to I-5-9, I-6-1 to I-6-9, I-7-1 to I-7-9, I-8-1 to I-8-9, and I-12-1 to I-12-9

### Step 1: General Synthesis of 2-(Cholesteryl-3-Yl)Oxyacetic Acid Analogues of Sterols

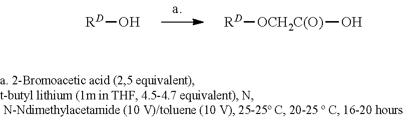

Cholesterol derivative R^{D} is conjugated to 2-bromoacetic acid through nucleophilic attack.

### Step 2: General Synthesis of Compound of Formula I

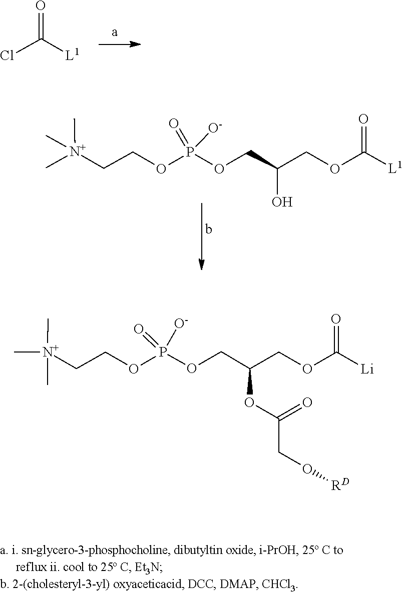

Fatty acid acyl chloride is attached to sn-glycero-3-phosphocholine. Then the 2-(cholesteryl-3-yl)oxyacetic acid obtained from step 1 is conjugated to the resulting compound to obtain Compound of Formula I.

### Example 11 General Synthesis of Compounds I-9-1 to I-9-9, I-13-1 to I-13-9, I-14-1 to I-14-9, I-15-1 to I-15-9, I-16-1 to I-16-9, I-17-1 to I-17-9

Cholesterol derivative R^{D} is reacted with succinic anhydride to form the corresponding succinates that are combined with various fatty acids to obtain further exemplary compounds of Formula I.

### Step 1: General Synthesis of Sterol Succinate

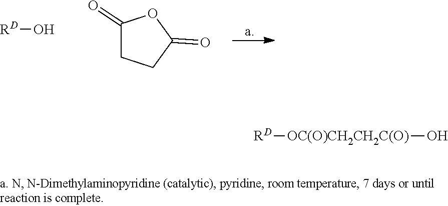

Cholesterol derivative R^{D} is reacted with succinic anhydride to obtain sterol succinate.

### Step 2: General Synthesis of Compound of Formula I

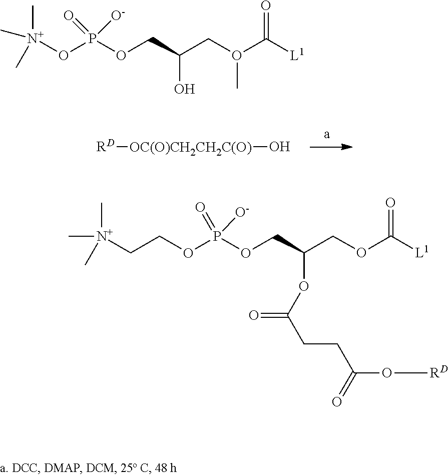

The fatty acid ester of sn-glycero-3-phosphocholine obtained from step 2 of Example 10 is reacted with the sterol succinate with a coupling agent to obtain exemplary compounds of Formula I.

### III. Biological Examples

### Example A: Preparation of Lipid Nanoparticle

Exemplary compounds of Formula I of the present disclosure were used to form lipid nanoparticles using the general method described below. The resulting lipid nanoparticles were characterized in terms of particle size (PS), polydispersity index (PDI), and encapsulation efficiency (ee %). PS, PDI, ee % were assessed again once the lipid particles have undergone a cycle of freeze-thaw. Results are shown in Table 2.

The LNPs were prepared by mixing lipids in ethanol with mRNA in an acidic aqueous buffer as previously described. In general, lipid excipients including ionizable lipid, phospholipid of the present disclosure, cholesterol, and PEG-DMG were dissolved in ethanol. The lipids were mixed, at a mRNA/total lipid weight/weight ratio of about 0.037, with mRNA solution prepared in pH 4.0 citrate buffer using a commercially available mixer (MIVM-1L, Holland Applied Technologies, Burr Ridge, IL). The freshly formed LNPs were stabilized by sequential dilution with phosphate buffer at pH 6.5, followed by HEPES buffer at pH 8.0. To concentrate the formulation, the diluted formulations were processed with tangential flow filtration (TFF) using PES hollow fiber membrane (100 kDa MWCO, Repligen, Waltham, MA), and further diafiltered with 10× volume of HEPES buffer and concentrated by centrifuging at 2000 RPM for 15-30 minutes in an Amicon® Ultra filtration tube with a 100 kD NMWL cellulose membrane (Millipore Sigma, USA). After filtering the formulation with 0.2 m PES filter, an in-process RNA concentration determination was performed using a RiboGreen® assay following vendor (Thermofisher Scientific, USA) protocol, and the formulation concentration was adjusted to the desired concentration by cryoprotectant addition. After sterile filtration, bulk formulation is aseptically filled into glass vials and stored frozen at −70° C.

The particle size and polydispersity index (PDI) were characterized using a Zen3600 (Malvern Instruments, with Zetasizer 7.1 software, Malvern, U.K). A volume of 50 μL of formulations were diluted into 950 μL of Tris buffer at pH=7.4 and equilibrated to 25° C. prior to analysis in a 1 mL cuvette using the following settings for measurement: material refractive index of 1.5, dispersant viscosity of 1.1 cP, and refractive index of 1.3. Each sample was analyzed for up to 30 runs. Encapsulation efficiency was calculated by determining the unencapsulated mRNA content by measuring the fluorescence intensity (Fi) upon the addition of RiboGreen (Molecular Probes, Eugene, OR, USA) to the LNP and comparing this value to the total fluorescence intensity (Ft) of the RNA content that is obtained upon lysis of the LNPs by 1% Triton X-100, where % encapsulation=(Ft−Fi)/Ft×100).

TABLE 2
Lipid particle characteristics

| | Bulk formulation | | | After 1 freeze-thaw cycle | | |
|---|---|---|---|---|---|---|
| | PS | | Encapsulation | PS | | Encapsulation |
| PCA* | (nm) | PDI | efficiency (ee %) | (nm) | PDI | efficiency (ee %) |
| I-2 | 86.6 | 0.139 | 99.3 | 76.3 | 0.204 | 99.3 |
| I-3 | 83.9 | 0.040 | 99.7 | 75.9 | 0.194 | 99.4 |
| I-4 | 83.5 | 0.057 | 98.2 | 78.9 | 0.160 | 99.3 |
| DSPC | 75.5 | 0.076 | 99.6 | 84.4 | 0.231 | 99.8 |

*PCA: Phosphatidylcholine analogue

Further exemplary compounds of Formula I were used to form LNPs with an ionizable cationic lipid L1. DSPC was used as a positive control. Results are shown in Table 3.

TABLE 3
Lipid particle characteristics

| | Bulk formulation | | | After 1 freeze-thaw cycle | | |
|---|---|---|---|---|---|---|
| LNP | PS (nm) | PDI | EE (%) | PS (nm) | PDI | EE (%) |
| L1 + I-5 | 70.93 | 0.106 | 96.3 | 71.36 | 0.15 | 93.6 |
| L1 + I-6 | 67.5 | 0.084 | 96.3 | 77.6 | 0.28 | 87.9 |
| L1 + I-7 | 71.45 | 0.047 | 92.9 | 74.61 | 0.12 | 92.4 |
| L1 + I-9 | 66.93 | 0.12 | 96.2 | 67.18 | 0.11 | 92.5 |
| L1 + I-8 | 66.86 | 0.09 | 96.9 | 69.39 | 0.12 | 94.4 |
| L1 + I-2 | 65.21 | 0.104 | 95.9 | 66.83 | 0.12 | 94.8 |
| L1 + DSPC | 65.81 | 0.113 | 94.6 | 67.47 | 0.17 | 94.2 |

As shown in the tables above, the compounds of Formula I were able to form uniform LNPs of particle size less than 100 nm, while maintaining a consistently high encapsulation efficiency.

### Example B: SiRNA Knock Down Assay in Mice

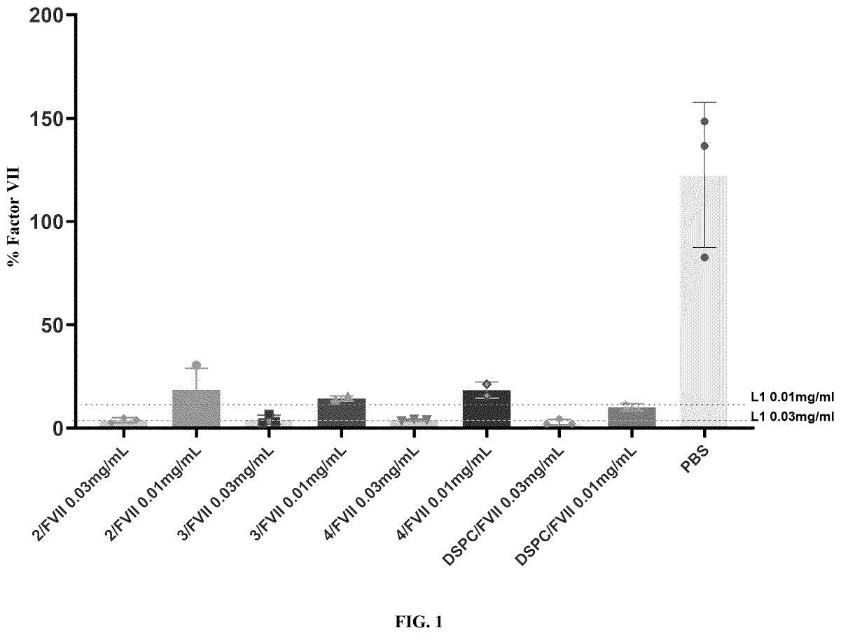

Lipid particle formulations using compounds I-2, I-3, or I-4 were then tested with FVII siRNA as exemplary nucleic acid to be encapsulated to assess capability of the compounds of Formula I to form functional lipid particles. The formed particles were then used in FVII mRNA knock down experiments. DSPC lipid nanoparticles were used as positive control. PBS was used as negative control. FVII mRNA knock down as represented by the reduction in FVII plasma protein (as a percentage of base value) at 48 hours after mice are administered intravenously with FVII siRNA encapsulated LNPs at dose levels of 0.01 and 0.03 mg/Kg of FVII siRNA. Results are shown in FIG. 1 LNPs formed with the compounds of Formula I of the present disclosure were able to knock down expression of the FVII target gene.

### Example C: Serum hEPO Expression in Mice and NHPs with Phosphatidyl Choline LNPs

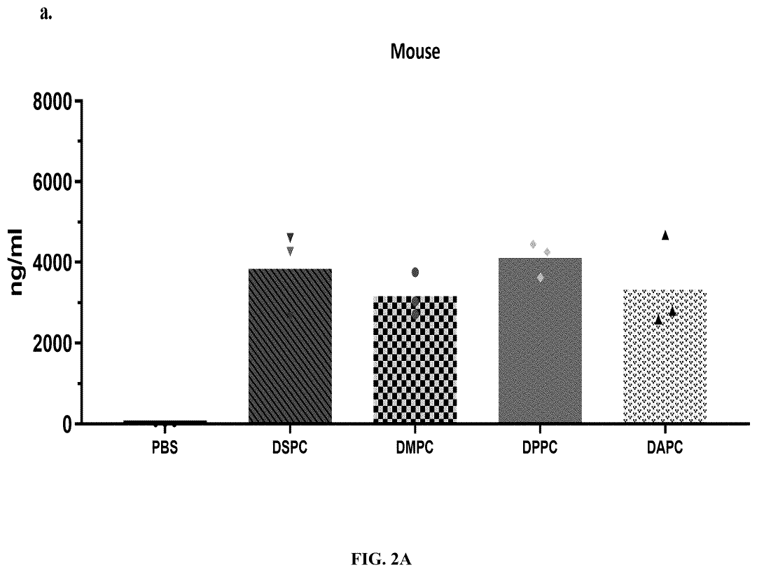

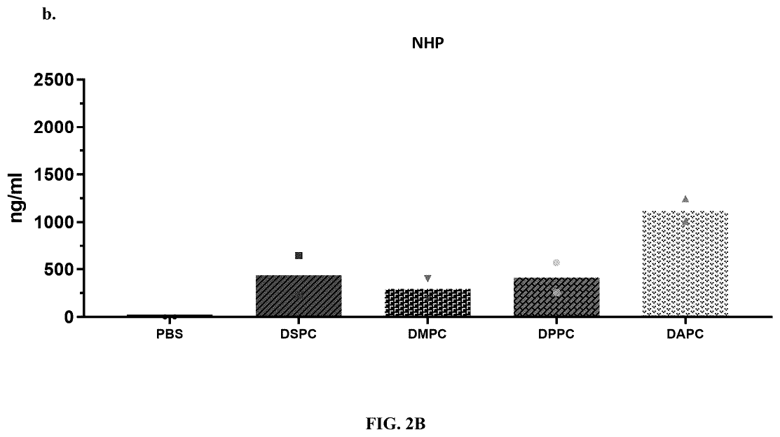

LNPs formulated with various phosphatidylcholine derivatives (DSPC, DMPC, DPPC and DAPC) using hEPO mRNA as the nucleic acid. The formulated LNPs were administered to mice and non-human primates (NHPs). hEPO protein expression was measured. Results are shown in FIG. 2. FIG. 2A shows Mouse serum hEPO protein expressions (ng/mL) at 6 hours post intravenous administration of hEPO mRNA in LNPs formulated with DSPC, DMPC, DPPC and DAPC each as helper lipids and L1 (di(pentadecan-8-yl) 4,4′-((((3-(dimethylamino)propyl)thio)carbonyl)azanediyl)dibutyrate) as the principal ionizable cationic lipid (iCL) at 0.3 mg/Kg doses. FIG. 2B shows hEPO expressions in NHP serum at 0.3 mg/kg dose of hEPO mRNA encapsulated with DSPC, DMPC, DPPC and DAPC each as helper lipids and L1 as the principal iCL.

In each case, relative to the negative control, hEPO protein expression was observed.

### Example D: Serum hEPO Expression in Mice and NHP Serum with LNPs of the Present Disclosure

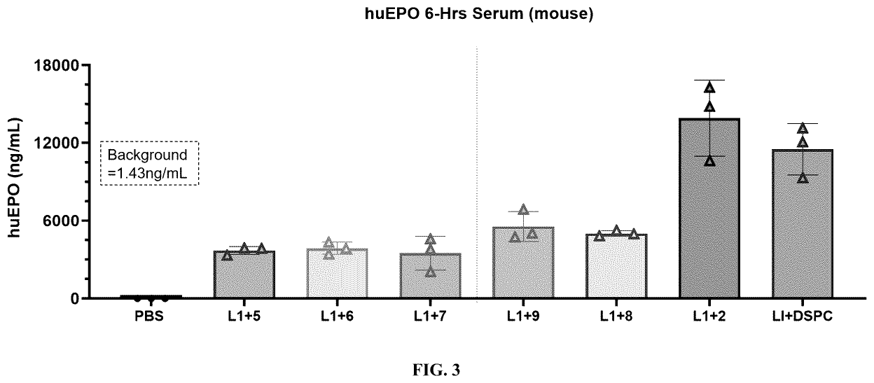

The LNPs encapsulating hEPO mRNA were formed with compounds I-2, I-5, I-6, I-7, I-8, or I-9 and the ionizable cationic lipid L1 using methods as described in Example A. The resulting LNPs were administered to mice. DSPC was used as a positive control. PBS was used as negative control. Mouse serum hEPO protein levels 6 h post administration of 0.3 mg/Kg of hEPO mRNA formulated in LNPs were assessed. Results are shown in FIG. 3. As shown, the LNPs of the present disclosure made with compounds of Formula I were able to deliver hEPO mRNA to the animal, leading to hEPO expression.

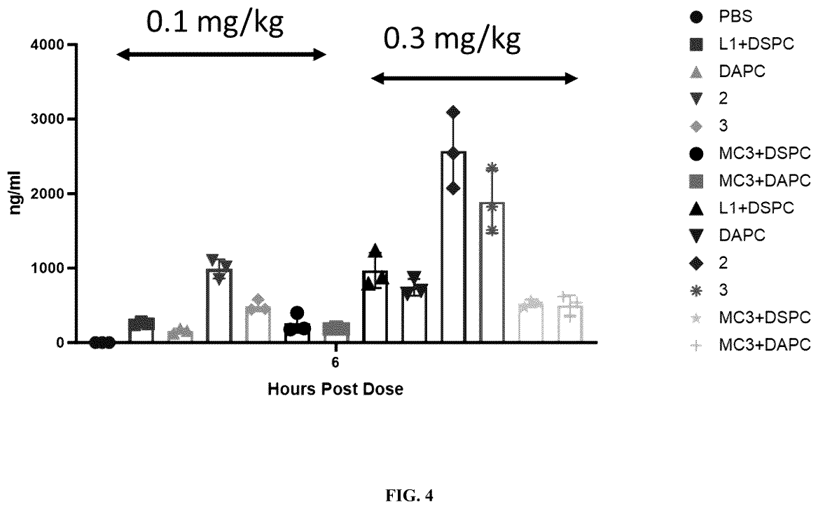

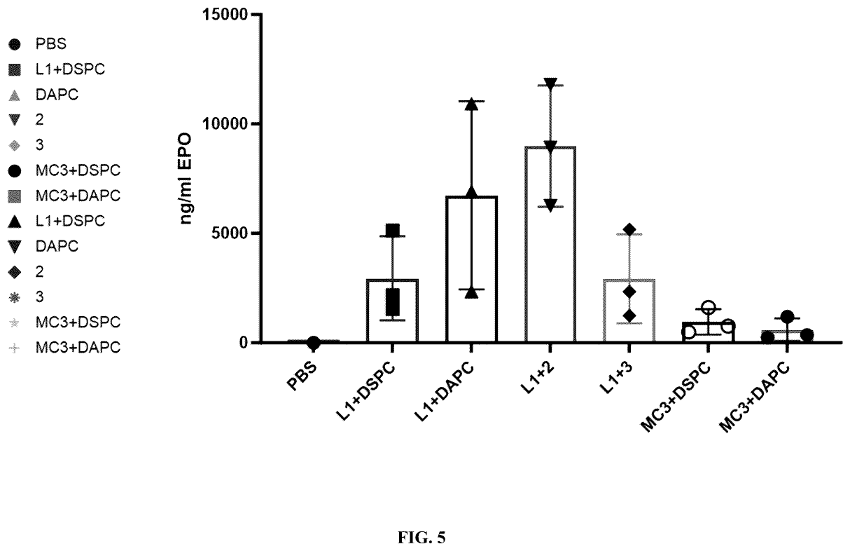

The LNPs encapsulating hEPO mRNA were formed with compound I-2 or I-3 and the ionizable cationic lipid L1 using methods as described in Example A. These LNPs were compared with hEPO mRNA containing LNPs made with DSPC+ionizable cationic lipid L1, DAPC+L1, DAPC, MC3+DSPC, MC3+DAPC. The LNPs were administered to mice at 0.1 or 0.3 mg hEPO/kg. hEPO expression was measured after 6 hours post intravenous administration. Results are shown in FIG. 4. The LNPs were also tested on NHP serum at 0.3 mg/kg. Results are shown in FIG. 5

As shown, the LNPs of the present disclosure, prepared using the compounds of Formula I were able to produce hEPO expression in a dose dependent fashion.

### Example E: Serum hEPO Expression in NHP Serum with LNPs of the Present Disclosure with Different Ionizable Cationic Lipids

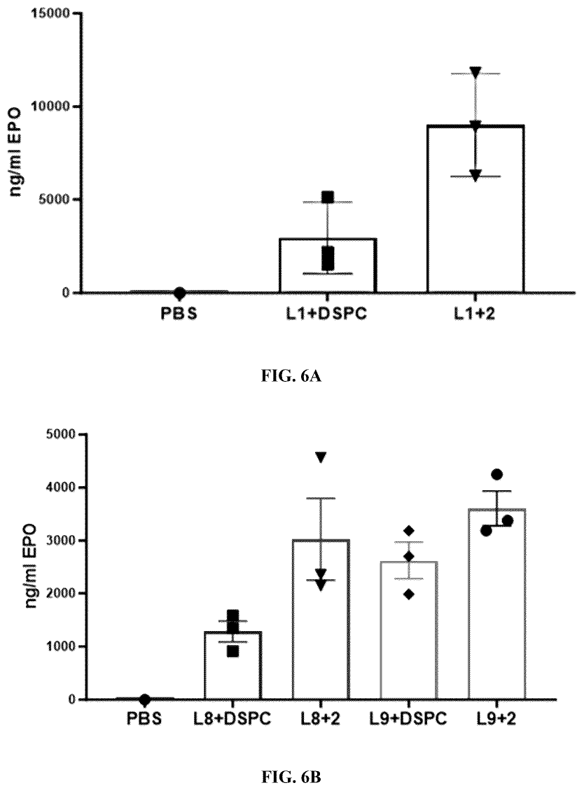

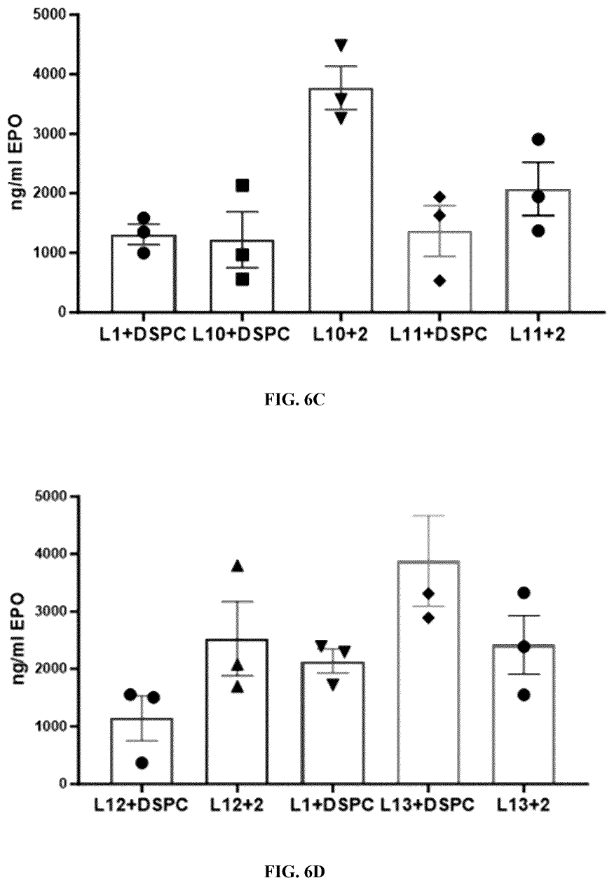

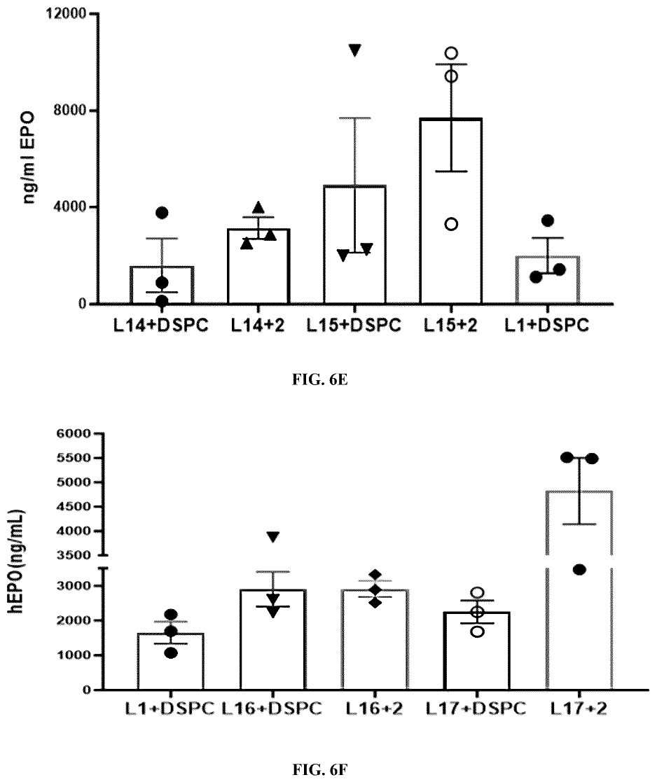

Compound I-2 was formulated into LNPs with different ionizable cationic lipids (L1, L8, L9, L10, L11, L12, L13, L14, L15, L16, L17) to encapsulate exemplary nucleic acid hEPO mRNA. DSPC was used as a positive control. PBS was used as negative control. The LNPs were administered to NHP ((n=3), 0.3 mg/Kg dose). Expression level of hEPO protein in NHP serum was measured. Results are shown in FIG. 6A-6F. As shown, compound I-2 containing LNPs were able to achieve higher expression level than DSPC containing LNPs.

The present disclosure provides reference to various embodiments and techniques. However, it should be understood that many variations and modifications can be made while remaining within the spirit and scope of the present disclosure. The description is made with the understanding that it is to be considered an exemplification of the claimed subject matter, and is not intended to limit the appended claims to the specific embodiments illustrated.

1. A compound of Formula I: 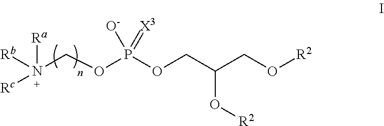
wherein
X¹, X², and X³ are each independently O or S,
R^{a}, R^{b}, and R^{c} are each independently H, or C₁₋₆ alkyl, or
R^{a} is H or C₁₋₆ alkyl, and R^{b} and R^{c} taken together with the N they are attached to form a 3- to 8-membered heterocyclyl, the heterocyclyl being optionally substituted with one or more C₁₋₃ alkyl,
n is 2 to 6,
either
(i) R¹ is 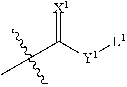
and R² is 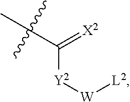
wherein
Y¹ is absent, O, or S,
L¹ is C₃₋₄₀ alkyl, C₃₋₄₀ alkenyl, or C₃₋₄₀ alkynyl,
either
(a) Y² is absent or C₁₋₄ alkylene, W is absent, O, or S, and L² is selected from C₃₋₄₀ alkyl, C₃₋₄₀ alkenyl, and C₃₋₄₀ alkynyl, optionally the alkyl, alkenyl, and alkynyl are branched, or
(b) Y² is C₁₋₄ alkylene, optionally substituted with oxo, W is absent, and L² is cholesterol or cholesterol derivative attached to Y² via an O atom of the cholesterol or via an O or S atom of the cholesterol derivative; or
(ii) R¹ is 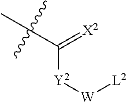
and R² is 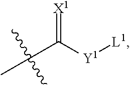
wherein
Y¹ is absent, O, or S,
L¹ is C₃₋₄₀ alkyl, C₃₋₄₀ alkenyl, or C₃₋₄₀ alkynyl,
Y² is C₁₋₄ alkylene, W is absent or carbonyl, and L² is cholesterol or cholesterol derivative attached to Y² via an O atom of the cholesterol or via an O or S atom of the cholesterol derivative.

2. The compound of claim 1, wherein the compound is of a compound of Formula I-I: 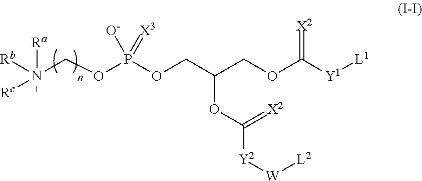
or a pharmaceutically acceptable salt thereof, wherein
X¹, X², and X³ are each independently O or S,
R^{a}, R^{b}, and R^{c} are each independently H, or C₁₋₆ alkyl, or
R^{a} is H or C₁₋₆ alkyl, and R^{b} and R^{c} taken together with the N they are attached to form a 3- to 8-membered heterocyclyl, the heterocyclyl being optionally substituted with one or more C₁₋₃ alkyl,
n is 2 to 6,
Y¹ is absent, O, or S,
L¹ is C₃₋₄₀ alkyl, C₃₋₄₀ alkenyl, or C₃₋₄₀ alkynyl,
+C₃₋₄₀ alkenyl, and C₃₋₄₀ alkynyl, optionally the alkyl, alkenyl, and alkynyl are branched, or
Y² is C₁₋₄ alkylene, optionally substituted with oxo, W is absent, and L² is cholesterol or cholesterol derivative attached to Y² via an O atom of the cholesterol or via an O or S atom of the cholesterol derivative.

3. The compound of claim 1, wherein
(i) X¹, X², and X³ are each independently O; or
(ii) R^{b} and R^{c} are each independently C₁₋₆ alkyl: or
(iii) R^{a} is H or C₁₋₆ alkyl; or
(iv) the compound comprises any combination of (i)-(iii); or
(v) R^{a}, R^{b} and R^{c} are each independently C₁₋₆ alkyl, C₁₋₃ alkyl, or methyl; or
(vi) R^{a} is C₁₋₆ alkyl or C₁₋₃ alkyl, optionally R^{a} is methyl, and R^{b} and R^{c} taken together with the N they are attached to form a 3- to 8-membered heterocyclyl, a 5- to 6-membered heterocyclyl, or a piperazinyl, each of which is optionally substituted with one or more C₁₋₃ alkyl; or
(vii) n is 2 to 4, or n is 2.

4.-15. (canceled)

16. The compound of claim 1, wherein
(i) Y¹ is absent, O, or S; or
(ii) L¹ is C₆₋₂₅ alkyl, C₁₀₋₂₀ alkyl, C₆₋₂₅ alkenyl, or C₆₋₂₅ alkynyl, optionally L¹ is branched; or
(iii) Y² is absent or C₁₋₄ alkylene, W is absent, O, or S, and L² is selected from C₃₋₄₀ alkyl, C₃₋₄₀ alkenyl, C₃₋₄₀ alkynyl, C₆₋₂₅ alkyl, C₆₋₂₅ alkenyl, C₆₋₂₅ alkynyl, C₁₀₋₂₀ alkyl, C₁₀₋₂₀ alkenyl, and C₁₀₋₂₀ alkynyl, optionally L² is C₁₀₋₂₀ alkyl, optionally L² is branched; or
(iv) Y² is C₁₋₄ alkylene or C₁₋₂ alkylene, W is absent, and L² is cholesterol or cholesterol derivative attached to Y² via an O atom of the cholesterol or cholesterol derivative, optionally wherein the cholesterol derivative is selected from Beta-sitosterol, stigmasterol, lanosterol, thiocholesterol, stigmastanol, campesterol, fucosterol, brassicasterol and ergosterol; or
(v) the compound comprises any combination of (i)-(iii); or
(vi) the compound comprises any combination of (i), (ii) and (iv).

17.-34. (canceled)

35. The compound of claim 1, wherein the compound is of Formula IA 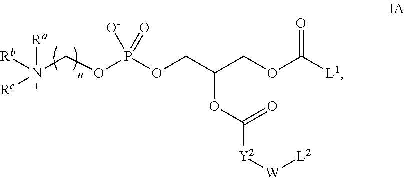
or a pharmaceutically acceptable salt thereof.

36. The compound of claim 35, wherein
(i) n is 2 to 4, optionally n is 2; or
(ii) Y² is absent and L² is selected from C₃₋₄₀ alkyl, C₃₋₄₀ alkenyl, C₃₋₄₀ alkynyl, C₆₋₂₅ alkyl, and C₁₀₋₂₀ alkyl; or
(iii) W is absent or O; or
(iv) Y² is C₁₋₄ alkylene and L² is cholesterol or cholesterol derivative attached to Y² via an O atom of the cholesterol or via an O or S atom of the cholesterol derivative, optionally wherein Y² is C₁₋₃ alkylene and L² is cholesterol; or
(v) L¹ is branched; or
(vi) R^{a}, R^{b}, and R^{c} are each independently C₁₋₃ alkyl; or
(vii) L¹ is C₆₋₂₅ alkyl, C₆₋₂₅ alkenyl, C₆₋₂₅ alkynyl, C₁₀₋₂₀ alkyl, or C₁₀₋₂₀ alkenyl, optionally wherein L¹ is branched; or
(viii) the compound comprises any combination of (i)-(vii).

37.-43. (canceled)

44. The compound of claim 36, wherein
(a) Y² is substituted with one oxo; or
(b) Y² is substituted with one oxo and L² is attached to Y² via an ester linkage.

45. (canceled)

46. The compound of claim 1, wherein the compound is of Formula IB 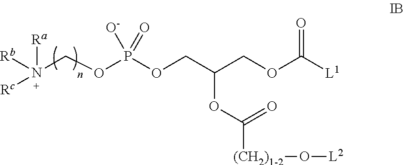
or a pharmaceutically acceptable salt thereof.

47. The compound of claim 46, wherein
(i) n is 2 to 4, optionally n is 2; or
(ii) L² is selected from C₃₋₄₀ alkyl, C₃₋₄₀ alkenyl, C₃₋₄₀ alkynyl, C₆₋₂₅ alkyl, and C₁₀₋₂₀ alkyl; or
(iii) L² is branched; or
(iv) R^{a}, R^{b}, and R^{c} are each independently C₁₋₃ alkyl; or
(v) L¹ is C₆₋₂₅ alkyl, C₆₋₂₅ alkenyl, C₆₋₂₅ alkynyl, C₁₀₋₂₀ alkyl, or C₁₀₋₂₀ alkenyl, optionally wherein L¹ is branched; or
(vi) the compound comprises any combination of (i)-(v).

48. The compound of claim 1, wherein the compound is of Formula IC 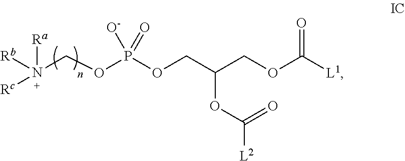
or a pharmaceutically acceptable salt thereof.

49. The compound of claim 48, wherein
(i) n is 2 to 4, optionally n is 2: or
(ii) L² is selected from C₃₋₄₀ alkyl, C₃₋₄₀ alkenyl, C₃₋₄₀ alkynyl, C₆₋₂₅ alkyl, and C₁₀₋₂₀ alkyl; or
(iii) L² is branched; or
(iv) the compound comprises any combination of (i)-(iii).

50.-54. (canceled)

55. The compound of claim 1, wherein the compound is of Formula ID 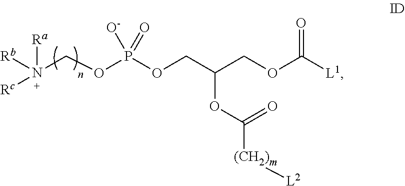
or a pharmaceutically acceptable salt thereof,
wherein m is 1 to 3.

56. The compound of claim 55, wherein
(i) L² is cholesterol or cholesterol derivative attached to Y² via an O atom of the cholesterol or cholesterol derivative; or
(ii) m is 1 or 2, optionally m is 1; or
(iii) R^{a}, R^{b}, and R^{c} are each independently C₁₋₃ alkyl; or
(iv) L¹ is C₆₋₂₅ alkyl, C₆₋₂₅ alkenyl, C₆₋₂₅ alkynyl, C₁₀₋₂₀ alkyl, or C₁₀₋₂₀ alkenyl, optionally wherein L¹ is branched; or
(v) the compound comprises any combination of (i)-(iv).

57.-65. (canceled)

66. The compound of claim 1, or a pharmaceutically acceptable salt thereof, wherein the compound is selected from the compounds of Table A, the compounds of Table B, and the compounds of Table C.

67.-68. (canceled)

69. A lipid composition comprising a nucleic acid and a compound of claim 1.

70. The lipid composition of claim 69, wherein the
(i) nucleic acid is selected from RNA, DNA, an siRNA, an mRNA, a self-replicating RNA, a DNA plasmid, and an antisense oligonucleotide; or
(ii) the nucleic acid is a mRNA or a self-replicating RNA comprising a coding region that encodes a protein selected from an enzyme, an antibody, an antigen, a receptor, a transporter, or a gene-editing enzyme, optionally wherein the gene-editing enzyme is selected from a TALEN, a CRISPR, a meganuclease, or a zinc finger nuclease; or
(iii) the lipid composition comprises liposomes, lipoplexes, or lipid nanoparticles; or
(iv) the lipid composition comprises (i) and (iii); or
(v) the lipid composition comprises (ii) and (iii).

71.-76. (canceled)

77. A lipid nanoparticle comprising a compound of Formula I of claim 1 or a salt thereof.

78. The lipid nanoparticle of claim 77, wherein
(i) the compound of Formula I or the salt thereof self-assembles to form the lipid nanoparticle; or
(ii) the average particle size of the lipid nanoparticle is less than about 100 nm; or
(iii) the lipid nanoparticle further comprises a nucleic acid, optionally wherein the nucleic acid is encapsulated in the lipid nanoparticle.

79.-82. (canceled)

83. The lipid nanoparticle of claim 78, wherein
(a) the nucleic acid is selected from RNA, DNA, an siRNA, an mRNA, a self-replicating RNA, a DNA plasmid, and an antisense oligonucleotide; or
(b) the nucleic acid is a mRNA or a self-replicating RNA comprising a coding region that encodes a protein selected from an enzyme, an antibody, an antigen, a receptor, a transporter, or a gene-editing enzyme, optionally wherein the gene-editing enzyme is selected from a TALEN, a CRISPR, a meganuclease, or a zinc finger nuclease.

84.-88. (canceled)

89. The lipid nanoparticle of claim 77, wherein
(i) the lipid nanoparticle further comprises a helper lipid selected from: dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), and phosphatidylcholine (PC); or
(ii) the lipid nanoparticle further comprises cholesterol; or
(iii) the lipid nanoparticle further comprises a polyethylene glycol (PEG)-lipid conjugate, optionally wherein the PEG-lipid is a PEG-DMG, optionally wherein the PEG-DMG is PEG2000-DMG: or
(iv) the lipid nanoparticle further comprises an ionizable cationic lipid; or
(v) the lipid nanoparticle further comprises any combination of (i)-(iv).

90.-95. (canceled)

96. The lipid nanoparticle of claim 89, wherein, based on the total amount of lipids in the lipid nanoparticle, the lipid nanoparticle comprises:
about 5 mol % to 15 mol % of a combined amount of the compound of claim 1 and the helper lipid, about 30 mol % to about 70 mol % of the ionizable cationic lipid, about 20 mol % to about 60 mol % of cholesterol, and about 0.5 mol % to about 5 mol % of the PEG-lipid conjugate; or
about 5 mol % to 10 mol % of a combined amount of the compound of claim 1 and the helper lipid, about 40 mol % to about 60 mol % of the ionizable cationic lipid, about 30 mol % to about 50 mol % of cholesterol, and about 0.5 mol % to about 3 mol % of the PEG-lipid conjugate; or
about 5 mol % to 12 mol % of a combined amount of the compound of claim 1 and the helper lipid, about 45 mol % to about 55 mol % of the ionizable cationic lipid, about 35 mol % to about 45 mol % of cholesterol, and about 0.5 mol % to about 3 mol % of PEG-lipid conjugate.

97.-98. (canceled)

99. The lipid nanoparticle of claim 77, wherein the lipid nanoparticle has a total lipid:nucleic acid weight ratio of:
about 50:1 to about 10:1;
about 40:1 to about 20:1;
about 35:1 to about 25:1;
about 32:1 to about 28:1; or
about 31:1 to about 29:1.

100.-103. (canceled)

104. A pharmaceutical composition comprising the compound of claim 1, or the lipid nanoparticle of claim 77, and a pharmaceutically acceptable excipient.

105. The pharmaceutical composition of claim 104, wherein the pharmaceutical composition is a lyophilized composition.

106. The pharmaceutical composition of claim 104, wherein the pharmaceutical composition comprises:
(i) a HEPES buffer at a pH of about 7.4, optionally wherein the HEPES buffer is at a concentration of about 7 mg/mL to about 15 mg/mL: or
(ii) about 2.0 mg/mL to about 4.0 mg/mL of NaCl; or
(iii) one or more cryoprotectants selected from sucrose, glycerol, or a combination of sucrose and glycerol; or
(iv) a combination of sucrose at a concentration of about 70 mg/mL to about 110 mg/mL and glycerol at a concentration of about 50 mg/mL to about 70 mg/mL: or
(v) any combination of (i)-(iii); or
(vi) any combination of (i), (ii), and (iv).

107.-111. (canceled)

112. A method of treating a disease in a subject in need thereof, comprising administering a therapeutically effective amount to the subject, the lipid nanoparticle of claim 77, or the pharmaceutical composition of claim 104, optionally wherein the pharmaceutical composition or lipid nanoparticle is administered intravenously or intramuscularly, optionally wherein the disease or disorder is associated with expression or overexpression of a gene, and/or wherein the disease or disorder is cancer or inflammatory disease, and/or wherein the disease is selected from the group consisting of central nervous system disorders, peripheral nervous system disorders, muscle atrophies, muscle dystrophies, immune disorder, cancer, renal disease, fibrotic disease, genetic abnormality, inflammation, and cardiovascular disorder.

113.-114. (canceled)

115. A method of expressing a protein or polypeptide in a target cell, comprising contacting the target cell with a lipid nanoparticle of claim 77, or the pharmaceutical composition of claim 104, optionally wherein the protein or polypeptide is an antigen, and expression of the antigen elicits an in vivo immunogenic response.

116-117. (canceled)
